(19) United States
(12) Patent Application Publication
(10) Pub. No.: US 2026/0201031 A1
(43) Pub. Date: Jul. 16, 2026

(54) BISPECIFIC CHIMERIC ANTIGEN RECEPTOR IMMUNE CELLS

(71) Applicants: ROSWELL PARK CANCER INSTITUTE CORPORATION HEALTH RESEARCH, INC., Buffalo, NY (US); H. LEE MOFFITT CANCER CENTER AND RESEARCH INSTITUTE, INC., Tampa, FL (US)

(72) Inventor: Marco L. DAVILA, Buffalo, NY (US)

(21) Appl. No.: 19/132,620

(22) PCT Filed: Nov. 22, 2023
(86) PCT No.: PCT/US2023/080983

§ 371 Date: May 23, 2025

Related U.S. Application Data
(60) Provisional application No. 63/384,911, filed on Nov. 23, 2022.

(51) Int. Cl. C07K 16/28; A61K 40/11; A61K 40/13; A61K 40/15; A61K 40/31; A61K 40/42

(52) U.S. Cl. CPC C07K 16/2803; A61K 40/11; A61K 40/13; A61K 40/15; A61K 40/31; A61K 40/4202; A61K 40/421; A61K 40/4217; A61K 40/4224; C07K 16/2818; C07K 16/2827; C07K 16/2851; C07K 16/2866; C07K 16/2896

(57) ABSTRACT

The present disclosure relates to compositions of bispecific chimeric antigen receptors and method of uses thereof.

### RELATED APPLICATION

This PCT application claims priority to, and the benefit of, U.S. Provisional Patent Application No. 63/384,911, filed Nov. 23, 2022, entitled “BISPECIFC CHIMERIC ANTIGEN RECEPTOR IMMUNE CELLS,” which is incorporated by reference herein in its entirety.

### REFERENCE TO SEQUENCE LISTING

The sequence listing submitted on Nov. 22, 2023, as an .xml file entitled “11390-009WO1.xml” created on Nov. 20, 2023, and having a file size of 324,119 bytes is hereby incorporated by reference pursuant to 37 C.F.R. § 1.52(e)(5).

### FIELD

The present disclosure relates bispecific chimeric antigen receptor compositions and methods of use thereof.

### BACKGROUND

It is believed that the human immune system is a resource for cancer therapy and that effective treatment can be developed once the components of the immune system are properly harnessed. Immunotherapy (sometimes called biological therapy, biotherapy, or biological response modifier therapy), uses the body's immune system, either directly or indirectly, to shrink or eradicate cancer has been studied for many years as an adjunct to conventional cancer therapy.

The development of chimeric antigen receptors (CAR) was a major advance in anti-cancer T cell therapy. CAR therapies comprises a single chain variable fragment (scFv) derived from an antibody fused to the signaling domains of an immune cell receptor. These engineered receptors are chimeric (peptide/polypeptides created through the joining of two or more genes that originally coded for separate proteins) by combining both antigen-binding and immune cell activating functions into one receptor. Induced expression of CARs on immune cells gives immune cells new functions to target a specific antigen. However, tumor heterogeneity and evasion of conventional CAR therapies remain an issue in treating cancer and preventing remission. Given the limitations of current CAR therapies, there is need to address the aforementioned problems mentioned above by developing strategies to prevent resistance or evasion to CAR therapies.

### SUMMARY

The present disclosure provides bispecific chimeric antigen receptor (CAR) compositions and methods of treating cancer.

In one aspect, disclosed herein are bispecific chimeric antigen receptor (CAR) polypeptides comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof. Also disclosed herein are bispecific CAR polypeptides of any preceding aspect, wherein the TAA binding domains comprise an anti-CD33 binding domain and an anti-CD123 binding domain; an anti-CD33 binding domain and an anti-CD99 binding domain; an anti-CLEC12A binding domain and an anti-CD99 binding domain; an anti-CLEC12A binding domain and an anti-CD123 binding domain; or an anti-CD123 binding domain and an anti-CD99 binding domain.

In one aspect, disclosed herein are isolated nucleic acids encoding the bispecific CAR polypeptide of any preceding aspect. Also, disclosed herein is an isolated nucleic acid encoding a bispecific chimeric antigen receptor (CAR) polypeptide comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof. For example, in one embodiment, the TAA binding domains comprise an anti-CD33 binding domain and an anti-CLEC12A binding domain (such as, for example a vector comprising a nucleic acid encoding a bispecific CAR polypeptide as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8).

In one aspect, disclosed herein is a vector comprising the isolated nucleic acid of any preceding aspect. Also, disclosed herein is a vector comprising a nucleic acid encoding a bispecific CAR comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof.

In some aspects, disclosed herein is a vector of any preceding aspect, wherein said vector includes, but is not limited to a DNA vector, a bacterial plasmid, a viral vector, a gene editing system, and a nanoparticle.

In one aspect, disclosed herein is a cell comprising the polypeptide, the nucleic acid, or the vector of any preceding aspect. Also, disclosed herein is a cell comprising a bispecific chimeric antigen receptor (CAR) polypeptide comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof.

In some aspects, disclosed herein, the bispecific CAR polypeptide of any preceding aspect is encoded by the nucleic acids sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some aspects, disclosed herein, the bispecific CAR polypeptide of any preceding aspect is encoded by the nucleic acid sequence as set forth in SEQ ID NO: 2, SEQ ID NO: 3, SEQ ID NO: 5, or SEQ ID NO: 7.

In some aspects, disclosed herein is a cell of any preceding aspect is selected from the group consisting of a αβT cell, γδT cell, a natural killer (NK) cell, a natural killer T (NKT) cell, a macrophage, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Also, disclosed herein the cell of any preceding aspect exhibits an anti-tumor immunity when a TAA binding domain of said cell binds to a TAA of a cancer cell.

In one aspect, disclosed herein are methods of treating, reducing, decreasing, inhibiting, ameliorating, and/or preventing a cancer and/or metastasis (such as, for example, acute myeloid leukemia (AML), testicular cancer, stomach cancer, urothelial cancer, lung cancer, and renal cancer) in a subject in need thereof, the method comprising administering to the subject a therapeutically effective amount of the cells of any preceding aspect. For example, disclosed herein are methods of treating, inhibiting, decreasing, reducing, ameliorating, and/or preventing a cancer and/or metastasis in a subject in need therein, the method comprising administering a therapeutically effective amount of a cell comprising a bispecific chimeric antigen receptor (CAR) polypeptide, wherein the polypeptide comprises at least two tumor associated antigen (TAA) binding domains selected from the group consisting of an anti-CD33 binding domain, an anti-CLEC12A binding domain, an anti-CD123 binding domain, an anti-CD99 binding domain, or any combination thereof. In some aspects, the cell used in the disclosed methods comprises a bispecific CAR comprising at least two tumor associated antigen (TAA) binding domains, wherein the TAA binding domain comprises an anti-CD33 binding domain and an anti-CLEC12A binding domain, an anti-CD33 binding domain and an anti-CD123 binding domain; an anti-CD33 binding domain and an anti-CD99 binding domain; an anti-CLEC12A binding domain and an anti-CD99 binding domain; an anti-CLEC12A binding domain and an anti-CD123 binding domain; or an anti-CD123 binding domain and an anti-CD99 binding domain.

In one aspect, disclosed herein are methods of treating, reducing, decreasing, inhibiting, ameliorating, and/or preventing a cancer and/or metastasis (such as, for example, acute myeloid leukemia (AML), testicular cancer, stomach cancer, urothelial cancer, lung cancer, and renal cancer) in a subject in need thereof, the method comprising administering to the subject a therapeutically effective amount of a bispecific chimeric antigen receptor (CAR) polypeptide of any preceding aspect. For example, disclosed herein are methods of treating, inhibiting, decreasing, reducing, ameliorating, and/or preventing a cancer and/or metastasis in a subject in need therein, the method comprising administering a therapeutically effective amount of a cell comprising a bispecific chimeric antigen receptor (CAR) polypeptide, wherein the polypeptide comprises at least two tumor associated antigen (TAA) binding domains selected from the group consisting of an anti-CD33 binding domain, an anti-CLEC12A binding domain, an anti-CD123 binding domain, an anti-CD99 binding domain, or any combination thereof. In some aspects, the cell used in the disclosed methods comprises a bispecific CAR comprising at least two tumor associated antigen (TAA) binding domains, wherein the TAA binding domain comprises an anti-CD33 binding domain and an anti-CLEC12A binding domain, an anti-CD33 binding domain and an anti-CD123 binding domain; an anti-CD33 binding domain and an anti-CD99 binding domain; an anti-CLEC12A binding domain and an anti-CD99 binding domain; an anti-CLEC12A binding domain and an anti-CD123 binding domain; or an anti-CD123 binding domain and an anti-CD99 binding domain.

Also disclosed herein are methods of treating, inhibiting, decreasing, reducing, ameliorating, and/or preventing a cancer and/or metastasis of any preceding aspect wherein the bispecific CAR polypeptide comprises at least two TAA binding domains comprising but not limited to any TAA of any preceding aspect, an anti-CD19 binding domain, an anti-TAG-72 binding domain, an anti-TIM3 binding domain, an anti-CD83 binding domain, an anti-B cell maturation antigen (BCMA) binding domain, an anti-CD22 binding domain, an anti-CD38 binding domain, an anti-NCAM1 binding domain, an anti-CD5 binding domain, an anti-CD70 binding domain, an anti-MET binding domain, an anti-L1CAM binding domain, an anti-CD44 binding domain, an anti-SLAMF7 binding domain, an anti-EGFR binding domain, an anti-EPHA2 binding domain, an anti-GPC3 binding domain, an anti-HER2 binding domain, an anti-mesothelin, an anti-PDCD1 binding domain, or any combinations thereof.

In some aspect, disclosed herein is a method of any preceding aspect comprising a bispecific CAR polypeptide of any preceding aspect encoded by the nucleic acids sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some aspects, disclosed herein, the bispecific CAR polypeptide of any preceding aspect is encoded by the nucleic acid sequence as set forth in SEQ ID NO: 2, SEQ ID NO: 3, SEQ ID NO: 5, or SEQ ID NO: 7.

Also, disclosed herein is a method of any preceding aspect comprising at least two TAA binding domains comprising an anti-CD33 binding domain and an anti-CLEC12A binding domain, an anti-CD33 binding domain and an anti-CD123 binding domain, an anti-CD33 binding domain and an anti-CD99 binding domain, an anti-CLEC12A binding domain and an anti-CD99 binding domain, an anti-CLEC12A binding domain and an anti-CD123 binding domain, or an anti-CD123 binding domain and an anti-CD99 binding domain.

In some aspects, method of any preceding aspect comprises a vector including, but not limited to a DNA vector, a bacterial plasmid, a viral vector, a gene editing system, and a nanoparticle.

Also, disclosed herein is a method of any preceding aspect comprising the cell is selected from the group consisting of a αβT cell, γδT cell, a natural killer (NK) cell, a natural killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Also, disclosed herein is a method of any preceding aspect further comprising administering an immunomodulating agent including, but not limited to PD-1 antibody, a PD-L1 antibody, a CTLA-4 antibody, or any combination thereof.

In some embodiments, the subject comprises a mammal including, but not limited to a human.

### BRIEF DESCRIPTION OF FIGURES

The accompanying figures, which are incorporated in and constitute a part of this specification, illustrate several aspects described below.

FIG. 1 shows the transduction efficiency of CD33/Clec12a scFv constructs.

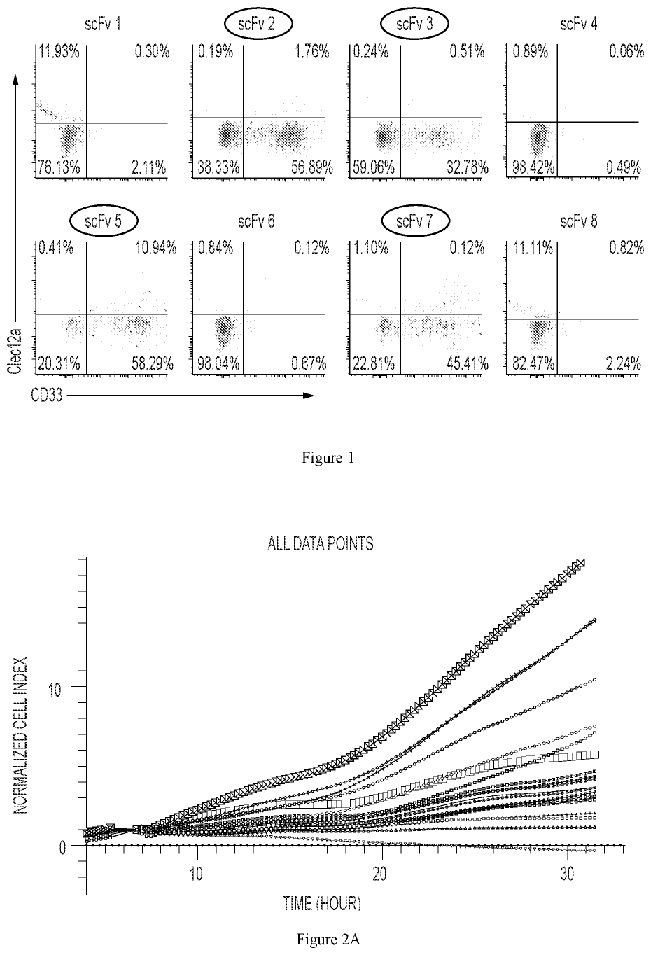

FIGS. 2A and 2B show the normalized cell index of CHO cells expressing CD33 or Clec12a CAR constructs being recognized by an scFv effector. FIG. 2A shows all data points using effectors scFv1, scFv2, scFv3, scFv4, scFv5, scFv6, scFv7, or scFv8. FIG. 2B shows the data points for the most efficient effectors: scFv3, scFv5, scFv2, and scFv7.

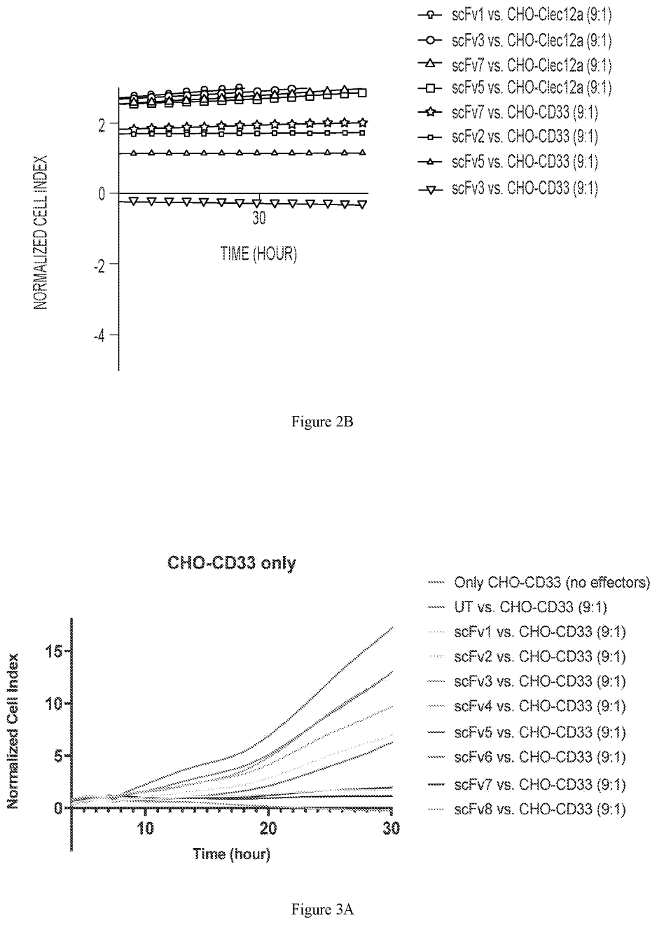

FIG. 3A show the normalized cell index of CHO-CD33 cells recognized by one of eight effectors: scFv1, scFv2, scFv3, scFv4, scFv5, scFv6, scFv7, or scFv8. FIG. 3B shows the normalized cell index of CHO-CD33 cells recognition by one of the top four effectors: scFv3, scFv5, scFv2, and scFv7.

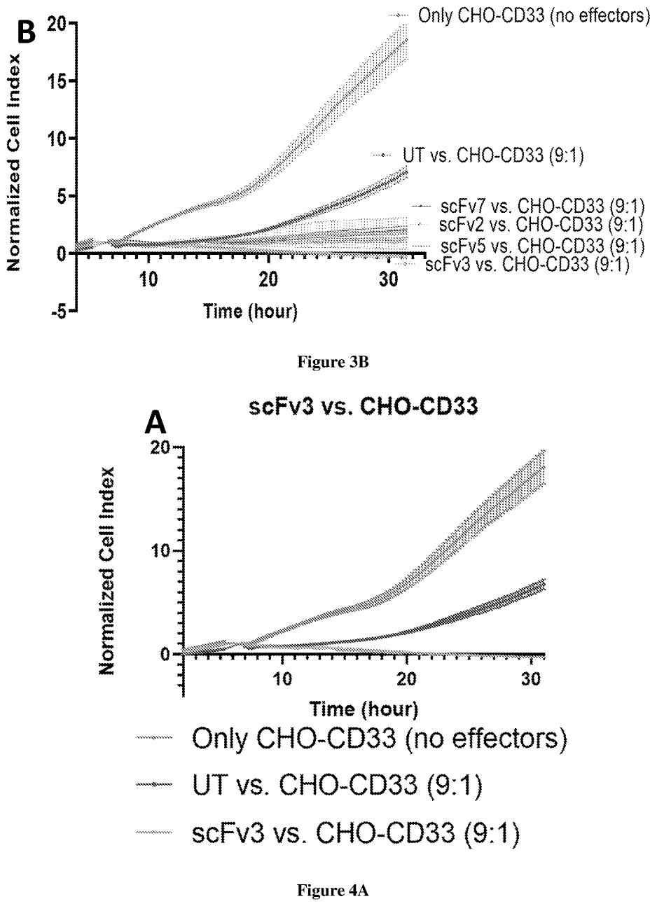

FIGS. 4A, 4B, 4C, and 4D show the individual graphs of CHO-CD33 cells recognized by one of the top four effectors. FIG. 4A shows the individual graph of CHO-CD33 cells recognized by scFv3. FIG. 4B shows the individual graph of CHO-CD33 cells recognized by scFv5. FIG. 4C shows the individual graph of CHO-CD33 cells recognized by scFv2. FIG. 4D shows the individual graph of CHO-CD33 cells recognized by scFv7.

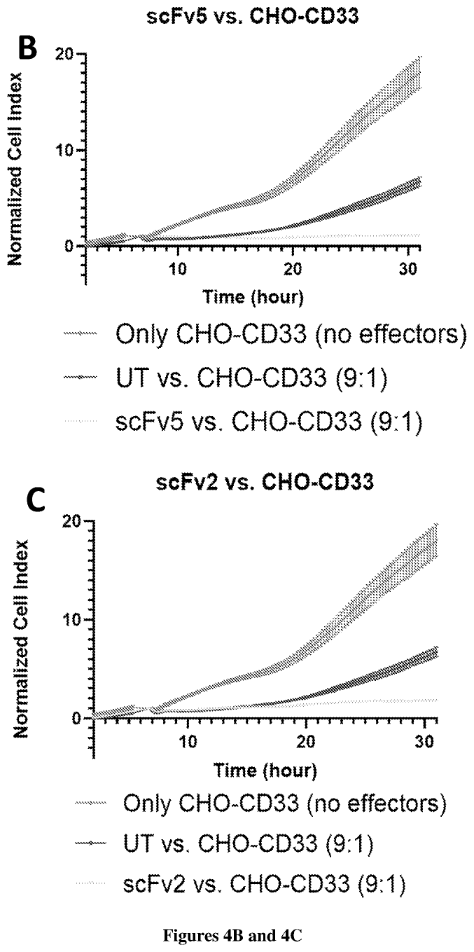

FIG. 5 shows the normalized cell index of CHO-Clec12a cells recognized by one of the eight effectors: scFv1, scFv2, scFv3, scFv4, scFv5, scFv6, scFv7, or scFv8.

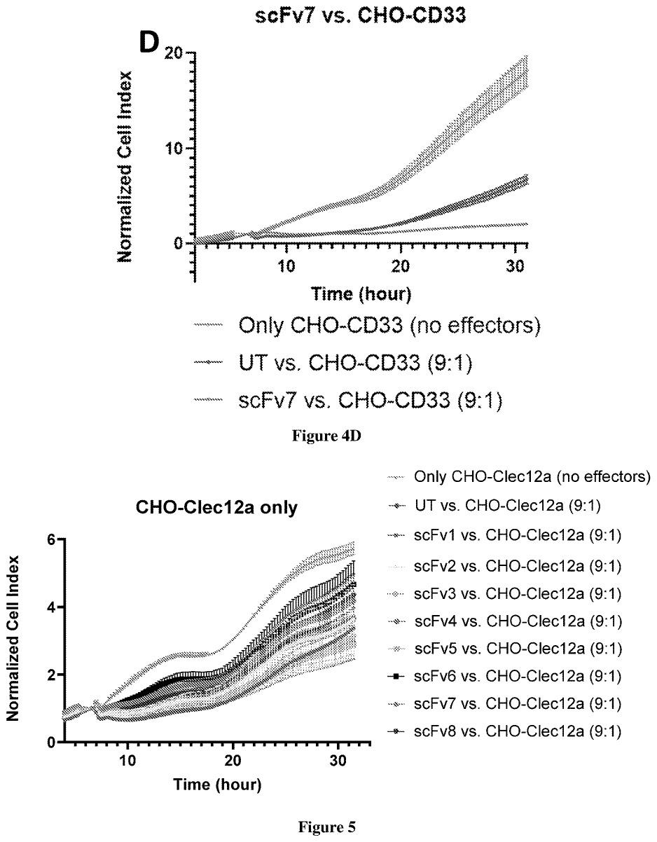

FIG. 6 shows the normalized cell index of CHO-Clec12a cells recognized by one of the top four effectors: scFv3, scFv5, scFv2, and scFv7.

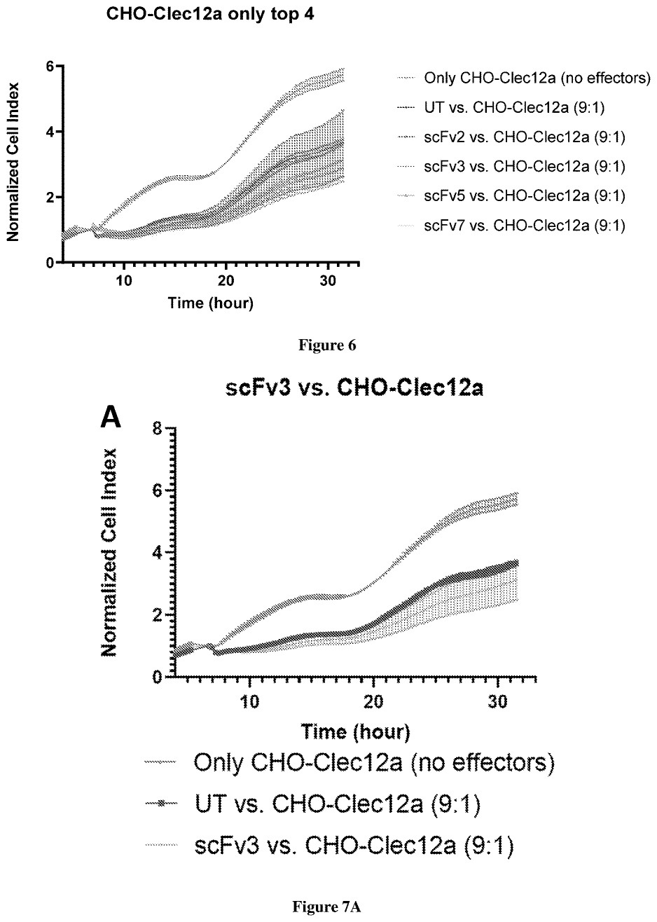

FIGS. 7A, 7B, 7C, and 7D show the individual graphs of CHO-Clec12a cells recognized by one of the top four effectors. FIG. 7A shows the individual graph of CHO-Clec12a cells recognized by scFv3. FIG. 7B shows the individual graph of CHO-Clec12a cells recognized by scFv5. FIG. 7C shows the individual graph of CHO-Clec12a cells recognized by scFv2. FIG. 7D shows the individual graph of CHO-Clec12a cells recognized by scFv7.

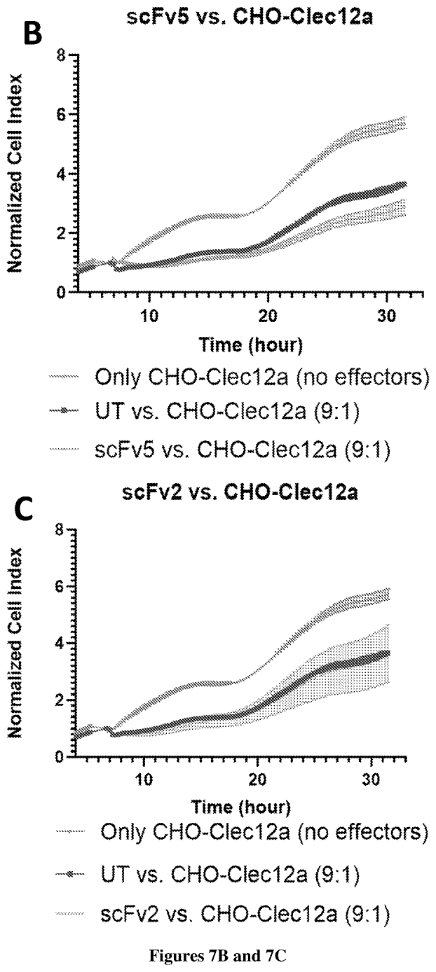

FIG. 8 shows the comparison of normalized cell indices in CHO-CD33 and CHO-Clec12a cells being recognized by scFv3.

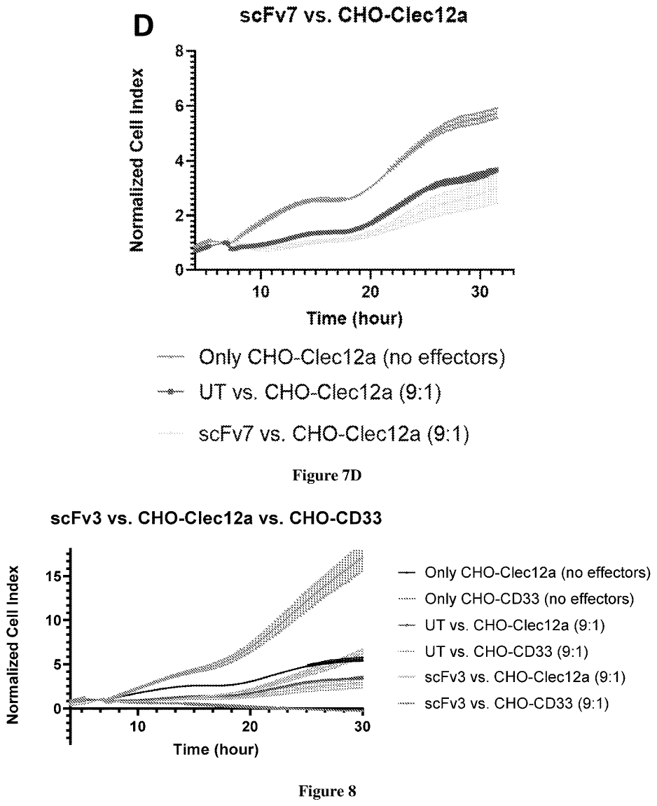

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” “treatment,” and grammatical variations thereof as used herein refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder and also includes include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “therapeutically effective” refers to the amount of the composition used is of sufficient quantity to ameliorate one or more causes or symptoms of a disease or disorder. Such amelioration only requires a reduction or alteration, not necessarily elimination.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

A “vector” refers to a DNA composition used as a vehicle to artificially carry foreign genetic material into another cell or tissue. A vector can be a bacterial plasmid, a viral vector, or a nanoparticle. A “bacterial plasmid” is a small extrachromosomal DNA molecule that can be incorporated into another cell that is physically separated from the chromosomal DNA and is easily replicated. A viral vector is a vehicle originally from a virus used to artificially carry foreign genetic material into another cell or tissue.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “chimeric antigen receptor” is an artificial T cell receptor used for immunotherapy. CAR are protein receptors that have been engineered to give T cells an enhanced ability to target a specific protein. CAR receptors are chimeric because the antigen binding and T cell activating functions have been combined into a single receptor.

Nanoparticles, as described herein, can be synthesized, or assembled via any suitable process. Preferably, the nanoparticles are assembled in a single step to minimize process variation. A single step process can include nanoprecipitation and self-assembly. The nanoparticles can be synthesized or assembled by dissolving or suspending the sunitinib in an organic solvent, preferably a solvent that is miscible in an aqueous solvent used for precipitation. In certain examples, acetonitrile is used as the organic solvent, but any suitable solvent can be used. Hydrophilic components are dissolved in a suitable aqueous solvent, such as water, 4 wt. % ethanol, or the like. The organic phase solution can be added drop wise to the aqueous phase solution to nanoprecipitate the sunitinib and allow self-assembly of the nanoparticle in the aqueous solvent.

As used herein, the term “agent” refers to a living organism or biological substance, such as a bacterium, virus, protozoan, parasite, fungus, chemical, or toxin, that can be designed to purposefully fulfill a biological function or action.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

A “T cell” refers to a type of lymphocyte that is one of the most important white blood cells of the immune system. T cells can be distinguished from other lymphocytes by the presence of a T-cell receptor (TCR) on their cell surface.

“Biocompatible” generally refers to a material and any metabolites or degradation products thereof that are generally non-toxic to the recipient and do not cause significant adverse effects to the subject.

A “control” is an alternative subject or sample used in an experiment for comparison purposes. A control can be “positive” or “negative.”

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Chimeric Antigen Receptor (CAR) Compositions

Chimeric antigen receptors (CARs) are engineered receptors that recognize and bind specific proteins expressed on cancer cells. CARs comprise a tumor associated antigen (TAA) binding domains that are complementary to the specific cancer proteins. These CARs can be incorporated into immune cells, including but not limited to T cells to allow these cells to recognize and kill target cancer cells quickly and efficiently. However recently, it has been shown that cancer cells and tumor can evade CAR-based immunotherapies due to cancer remission, and unregulated growth, invasion, and metastasis. Therefore, there is a need to develop CARs with increased potency to improve the expansion, activation, persistence, and tumor-eradicating activities of CAR immune cells.

The present disclosure provides compositions of bispecific chimeric antigen receptors (CARs) comprising combinations of tumor associated antigen (TAA) binding domains.

In one aspect, disclosed herein is a bispecific chimeric antigen receptor (CAR) polypeptide comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof. In some embodiments, the bispecific CAR polypeptide comprises at least two TAA binding domains consisting of, but not limited to any TAA of any preceding aspect, an anti-CD19 binding domain, an anti-TAG-72 binding domain, an anti-TIM3 binding domain, an anti-CD83 binding domain, an anti-B cell maturation antigen (BCMA) binding domain, an anti-CD22 binding domain, an anti-CD38 binding domain, an anti-NCAM1 binding domain, an anti-CD5 binding domain, an anti-CD70 binding domain, an anti-MET binding domain, an anti-L1CAM binding domain, an anti-CD44 binding domain, an anti-SLAMF7 binding domain, an anti-EGFR binding domain, an anti-EPHA2 binding domain, an anti-GPC3 binding domain, an anti-HER2 binding domain, an anti-mesothelin, an anti-PDCD1 binding domain, or any combinations thereof.

For example, in one embodiment, the TAA binding domains comprise an anti-CD33 binding domain and an anti-CLEC12A binding domain (such as, for example, a bispecific AR polypeptide wherein the anti-CD33 binding domain comprises heavy chain complementarity determining regions 1 (CDR1), CDR2, and CD3 wherein the CDR1 sequence of the V_{H} domain comprises the amino acid sequence GFTFSNYG (SEQ ID NO:137), GYTFTSYW (SEQ ID NO:138), or GFSLSRYS (SEQ ID NO:139), wherein the CDR2 sequence of the V_{H} domain comprises the amino acid sequence ISSGGGDT (SEQ ID NO:140), IHPSDSET (SEQ ID NO:141), or IWGGGYT (SEQ ID NO:142), wherein the CDR3 sequence of the V_{H} domain comprises the amino acid sequence ARDYGGTWDYFDY (SEQ ID NO:143), AREEGQLGHGGAMDY (SEQ ID NO:144), or ARYIDSSGYDY (SEQ ID NO: 145), wherein the CDR1 sequence of the VL comprises the amino acid sequence QDISKY (SEQ ID NO: 146), QTVNDD (SEQ ID NO: 147), SSVSY (SEQ ID NO: 148), or ENYSY (SEQ ID NO:149), wherein the CDR2 sequence of the V domain comprises the amino acid sequence YTSx (SEQ ID NO: 150), YVSx (SEQ ID NO: 151), DTSx (SEQ ID NO:152), or NAKx (SEQ ID NO: 153), wherein the CDR3 sequence of the V₁ domain comprises the amino acid sequence QQGDTFPWT (SEQ ID NO: 154), QQDYSSPYT (SEQ ID NO:155), QQWSSNPLT (SEQ ID NO:156), or QHHYGTPYT (SEQ ID NO:157), or any combination thereof.

In some embodiments, the anti-CD123 scFv is derived from hybridoma 3F5, 4E10, 121H5, 15A12, 17E7, 12H1, or combinations thereof. In some embodiments, the anti-CD123 scFv can comprise a variable heavy (VH) domain having CDR1, CDR2 and CDR3 sequences and a variable light (V_{L}) domain having CDR1, CDR2 and CDR3 sequences. For example, in some embodiments, the CDR1 sequence of the VH domain comprises the amino acid sequence GYTFTDYN (SEQ ID NO: 158), CDR2 sequence of the VH domain comprises the amino acid sequence INPNNGGT (SEQ ID NO: 159), CDR3 sequence of the VH domain comprises the amino acid sequence ARKGYGGNYDYFDY (SEQ ID NO: 160), CDR1 sequence of the V_{L} Comprises the amino acid sequence QSIGTS (SEQ ID NO: 161), CDR2 sequence of the Vt, domain comprises the amino acid sequence YASx (SEQ ID NO: 162), and CDR3 sequence of the Vl_domain comprises the amino acid sequence QQSNSWPYT (SEQ ID NO: 163). In some embodiments, the CDR1 sequence of the V_{H} domain comprises the amino acid sequence GFNIKDTY (SEQ ID NO: 164) or GFSLSTYGMG (SEQ ID NO: 165), the CDR2 sequence of the VH domain comprises the amino acid sequence IDPANGNT (SEQ ID NO: 166) or iYWDDDK (SEQ ID NO: 167), the CDR3 sequence of the VH domain comprises the amino acid sequence ALYYYGGSLDY (SEQ ID NO: 168) or AQSLIYDGYYGFAY (SEQ ID NO: 169), the CDR1 sequence of the V_{L} comprises the amino acid sequence QSLLYSGNQKNY (SEQ ID NO: 170), the CDR2 sequence of the VL domain comprises the amino acid sequence WASx (SEQ ID NO: 171), and the CDR3 sequence of the VL domain comprises the amino acid sequence QQYYSYPRT (SEQ ID NO: 172)In some embodiments, the CDR1 sequence of the VH domain comprises the amino acid sequence GYTFTYYG (SEQ ID NO: 173), the CDR2 sequence of the V_{H} domain comprises the amino acid sequence INTYSGVP (SEQ ID NO: 174), the CDR3 sequence of the VH domain comprises the amino acid sequence ARXVIYYSDLYGMDY (SEQ ID NO: 175), the CDR1 sequence of the V_{L} comprises the amino acid sequence QSIVHSNGDTY (SEQ ID NO: 176), the CDR2 sequence of the V_{L} domain comprises the amino acid sequence KVSx (SEQ ID NO: 177), and the CDR3 sequence of the V_{L} domain comprises the amino acid sequence FQGSHVPWT (SEQ ID NO: 178). In some embodiments, the CDR1 sequence of the VH domain comprises the amino acid sequence GYTFSSYW (SEQ ID NO: 179) or GYTLTTYL (SEQ ID NO: 180), the CDR2 sequence of the VH domain comprises the amino acid sequence INPSSGYT (SEQ ID NO: 181) or INPNSGSS (SEQ ID NO: 182), the CDR3 sequence of the VH domain comprises the amino acid sequence ARDGNYDHWYFDV (SEQ ID NO: 183) or AIRHYGGSLFDY (SEQ ID NO: 184), the CDR1 sequence of the V_{L} comprises the amino acid sequence QDINSY (SEQ ID NO: 185) or QSLLNSRTRKNY (SEQ ID NO: 186), the CDR2 sequence of the VL domain comprises the amino acid sequence WASx (SEQ ID NO: 171), or RANx (SEQ ID NO: 187), and the CDR3 sequence of the VL domain comprises the amino acid sequence LQYDELLT (SEQ ID NO: 188) or EQSYNLFT (SEQ ID NO: 189). In some embodiments, the some embodiments, the CDR1 sequence of the VH domain comprises the amino acid sequence GYTFTIDYN (SEQ ID NO: 158), GFNIKDTY (SEQ ID NO: 164), GFSLSTYGMG (SEQ ID NO: 165), GYTFTYYG (SEQ ID NO: 173), GYTFSSYW (SEQ ID NO: 179), or GYTLTTYL (SEQ ID NO: 180); the CDR2 sequence of the V_{H} domain comprises the amino acid sequence INPNNGGT (SEQ ID NO: 159), IDPANGNT (SEQ ID NO: 166), IYWDDDK (SEQ ID NO: 167), INTYSGVP (SEQ ID NO: 174), INPSSGYT (SEQ ID NO: 181), or INPNSGSS (SEQ ID NO: 182); the CDR3 sequence of the VH domain comprises the amino acid sequence ARKGYGGNYDYPDY (SEQ ID NO: 160), ALYYYGGSLDY (SEQ ID NO: 168), AQSLIYDGYYGFAY (SEQ ID NO: 169), ARWIYYSDLYGMDY (SEQ ID NO: 175), ARDGNYDHWYFDV (SEQ ID NO: 183), or AIRHYGGSLFDY (SEQ ID NO: 184); the CDR1 sequence of the VL comprises the amino acid sequence QSIGTS (SEQ ID NO: 161), QSLLYSGNQKNY (SEQ ID NO: 170), QSIVHSNGDTY (SEQ ID NO: 176), QDINSY (SEQ ID NO: 185), or QSLLNSRTRKNY (SEQ ID NO: 186); the CDR2 sequence of the VL domain comprises the amino acid sequence YAS, WAS, KVS, or RAN; the CDR3 sequence of the VL domain comprises the amino acid sequence QQSNSWPYT (SEQ ID NO: 163), QQYYSYPRT (SEQ ID NO: 172), FQGSHVPWT (SEQ ID NO: 178), LQYDELLT (SEQ ID NO: 188), or EQSYNLFT (SEQ ID NO: 189); or any combination thereof.

In some embodiments, the ani-CD99 region of the disclosed antibody or CAR is derived from hybridoma 1 H3, 4C5, 9G12, 3C7, 2F11, 4D5, 4F4, 6A10, or combinations thereof. In some embodiments, the ani-CD99 region (e.g. scFv) can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFDIKDTY (SEQ ID NO: 190), TYAMY (SEQ ID NO: 191), TFWM (SEQ ID NO: 192), or TFWMQ (SEQ ID NO: 193); the CDR2 sequence of the V_{H} domain comprises the amino acid sequence IDPANGDT (SEQ ID NO: 194), RIRSKVNNYATYYADSVKDRFT (SEQ ID NO: 195), or TIYPGDDDTRYTQKFKGRAT (SEQ ID NO:196); the CDR3 sequence of the V_{H} domain comprises the amino acid sequence ARRGGLS (SEQ ID NO: 197), DPMDY (SEQ ID NO: 198), or SGYERGPYYFDS (SEQ ID NO: 199), or SGYERGPYYF (SEQ ID NO: 200); the CDR1 sequence of the V_{L} comprises the amino acid sequence GNIHNY (SEQ ID NO:201), GSSKSLLHSNGNTYLY (SEQ ID NO: 202), KSSQSLLCRSNQKNYLA (SEQ ID NO: 203), or KSSQSLLYRSNQKNYLA (SEQ ID NO: 204); the CDR2 sequence of the V_{L} domain comprises the amino acid sequence NAKx (SEQ ID NO: 153), RVSNLAS (SEQ ID NO:205), or WASTRES (SEQ ID NO: 206); and the CDR3 sequence of the V_{L} domain comprises the amino acid sequence QHFWSTPWT (SEQ ID NO:207), MQHLEYPYT (SEQ ID NO: 208), or QQYYSYPLT (SEQ ID NO: 209).

In some embodiments, the anti-CLEC12A region of the disclosed antibody or CAR is derived from hybridoma 1 F3, 1 F8, 1G3, 2A10, 3F12, 4E3, 4E10, 5B2, 5F10, 6C7, 9A2, 11C7, 11 H 1, 12D6, or combinations thereof. In some embodiments, the anti-CLEC12A region (e.g. scFv) can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFTFSSFA (SEQ ID NO: 210), SFAVS (SEQ ID NO: 211), or SHDMS (SEQ ID NO: 212); the CDR2 sequence of the V_{H} domain comprises the amino acid sequence ISSGGAYT (SEQ ID NO: 213) or TISSGGAYTFYKDSVKGRFT (SEQ ID NO: 214), or YISGGGTNIYYSDTVKGRFT (SEQ ID NO: 215); the CDR3 sequence of the V_{H} domain comprises the amino acid sequence ARHSGYDGYYLYAMDY (SEQ ID NO: 216), HSGYDGYYLYAM DY (SEQ ID NO: 217), or PNYNYGGSWFAY (SEQ ID NO: 218); the CDR1 sequence of the V_{L} comprises the amino acid sequence SSVHY (SEQ ID NO: 219), ASSSVHYMH (SEQ ID NO: 220), or SASSSVHYMH (SEQ ID NO: 221); the CDR2 sequence of the Vt domain comprises the amino acid sequence DTSx (SEQ ID NO: 152) or DTSKLAS (SEQ ID NO: 222); and the CDR3 sequence of the V_{L} domain comprises the amino acid sequence QQWTSNPPT (SEQ ID NO: 223).

Nucleic acid sequences encoding a CAR disclosed herein or succeeding aspect, and region thereof, can be obtained using recombinant method known in the art, such as, for example by screening libraries from cells expressing the gene, by deriving the gene from a vector known to induce the same, or by isolating directly from cells and tissues containing the same, using standard techniques. Alternatively, the gene of interest can be produced synthetically, rather than cloned. In one aspect, disclosed herein are nucleic acid encoding any of the bispecific CAR polypeptides disclosed herein. For example, disclosed herein are bispecific CAR polypeptides encoded by a nucleic acids sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

In one aspect, disclosed herein is an isolated nucleic acid encoding a bispecific chimeric antigen receptor (CAR) polypeptide comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof. In some embodiments, the isolated nucleic acid sequence encodes the bispecific CAR polypeptide comprising at least two TAA binding domains consisting of, but not limited to any TAA of any preceding aspect, an anti-CD19 binding domain, an anti-TAG-72 binding domain, an anti-TIM3 binding domain, an anti-CD83 binding domain, an anti-B cell maturation antigen (BCMA) binding domain, an anti-CD22 binding domain, an anti-CD38 binding domain, an anti-NCAM1 binding domain, an anti-CD5 binding domain, an anti-CD70 binding domain, an anti-MET binding domain, an anti-L1CAM binding domain, an anti-CD44 binding domain, an anti-SLAMF7 binding domain, an anti-EGFR binding domain, an anti-EPHA2 binding domain, an anti-GPC3 binding domain, an anti-HER2 binding domain, an anti-mesothelin, an anti-PDCD1 binding domain, or any combinations thereof.

Expression of nucleic acids encoding CARs is typically achieved by operably linking a nucleic acid encoding the CAR polypeptide to a promoter and incorporating the construct into an expression vector. Typical expression vectors contain transcription and translation terminators, initiation sequences, reporter genes, selection genes, and promoters used for regulating the expression of the desired CAR nucleic acid sequence.

In another aspect, disclosed herein are vectors comprising any of the nucleic acids disclosed herein. For example, disclosed herein are vectors comprising a nucleic acid encoding a bispecific CAR comprising at least two TAA binding domains, wherein the TAA binding domains are selected from the group comprising an anti-CD33 binding domain, an anti-CLEC12A binding domain, an anti-CD123 binding domain, an anti-CD99 binding domain, or any combinations thereof, or any combinations thereof. In some embodiments, the vector comprises at least two TAA binding domains consisting of, but not limited to any TAA of any preceding aspect, an anti-CD19 binding domain, an anti-TAG-72 binding domain, an anti-TIM3 binding domain, an anti-CD83 binding domain, an anti-B cell maturation antigen (BCMA) binding domain, an anti-CD22 binding domain, an anti-CD38 binding domain, an anti-NCAM1 binding domain, an anti-CD5 binding domain, an anti-CD70 binding domain, an anti-MET binding domain, an anti-L1CAM binding domain, an anti-CD44 binding domain, an anti-SLAMF7 binding domain, an anti-EGFR binding domain, an anti-EPHA2 binding domain, an anti-GPC3 binding domain, an anti-HER2 binding domain, an anti-mesothelin, an anti-PDCD1 binding domain, or any combinations thereof.

For example, in one embodiment, the TAA binding domains comprise an anti-CD33 binding domain and an anti-CLEC12A binding domain (such as, for example a vector comprising a nucleic acid encoding a bispecific CAR polypeptide as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8). Also disclosed herein are vectors a nucleic acid encoding a bispecific CAR comprising at least two tumor associated antigen (TAA) binding domains, wherein the TAA binding domain comprises an anti-CD33 binding domain and an anti-CD123 binding domain; an anti-CD33 binding domain and an anti-CD99 binding domain; an anti-CLEC12A binding domain and an anti-CD99 binding domain; an anti-CLEC12A binding domain and an anti-CD123 binding domain; or an anti-CD123 binding domain and an anti-CD99 binding domain.

The disclosed nucleic acid can be cloned into a number of different vectors. For example, the nucleic acid can be cloned into a vector including, but not limited to a plasmid, a phagemid, a phage derivative, a phage, an animal virus, a cosmid, or a CRISPR-Cas gene editing system. Vectors of particular interest include expression vectors, replication vectors, probe generation vectors, and sequencing vectors.

Furthermore, an expression vector may be delivered into a cell in the form of a viral vector. Viral vector technology is well known in the art and is described in various virology and molecular biology texts, including but not limited to Sambrook et al. (2001, Molecular Cloning: A Laboratory Manual, Cold Spring Harbor Laboratory, New York).Viruses that are useful as vectors include, but are not limited to retroviruses, adenoviruses, adeno-associated viruses, herpes viruses, and lentiviruses. In general, a suitable viral vector contains an origin of replication functional in at least one organism, a promoter sequence, convenient and numerous restriction endonuclease sites, one or more reporter genes, and one or more selectable marker.

In cases where non-viral delivery systems are utilized, chemical delivery of the vector can be utilized. Chemical means of delivering a nucleic acid or expression vector into a host cell includes colloidal dispersion systems to include, but are not limited to macromolecule complexes, nanocapsules or nanoparticles, microspheres, beads, and lipid-based systems including oil-in-water emulsions, micelles, mixed micelles, and liposomes.

In some embodiments, the vector is DNA vector, a bacterial plasmid, a viral vector, a gene editing system, or a nanoparticle. In some embodiments, the vector is a lentiviral vector.

Immune cells, particularly immune effector cells, can be obtained from a number of sources, including peripheral blood mononuclear cells, bone marrow, lymph node tissues, spinal cord blood, umbilical cord blood, thymus tissue, tissue from an infection site, ascites, pleural effusion, spleen tissue, and tumors. Once obtained, immune cells can receive delivery of an expression vector to induce expression of said CARs in an immune cell population.

In another aspect, disclosed herein is a cell comprising any of the polypeptides, nucleic acids, or the vectors disclosed herein. In one embodiment, the cell is selected from the group consisting of a αβT cell, γδT cell, a natural killer (NK) cell, a natural killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof. In another embodiment, the cell exhibits an anti-tumor immunity when a TAA binding domain of said cell binds to a TAA of a cancer cell.

In some aspects, disclosed herein, the cell of any preceding aspect comprises any of the polypeptides, nucleic acids, or the vectors disclosed herein, wherein the nucleic acid sequence comprises SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some aspects, disclosed herein, the cell of any preceding aspect comprises any of the polypeptides, nucleic acids, or the vectors disclosed herein, wherein the nucleic acid sequence comprises SEQ ID NO: 2, SEQ ID NO: 3, SEQ ID NO: 5, or SEQ ID NO: 7.

### Methods of Treating Cancer

Immune effector cells expressing the disclosed CARs, nucleic acid, or vectors of any preceding aspect can elicit an anti-tumor response against any TAA-expressing cancer cell. This anti-tumor response incorporates recruiting more immune cells, including but not limited to cytotoxic T cells, helper T cells, memory T cells, natural killer (NK) cells, natural killer T (NKT) cells, and B cells, which promote the decrease of tumor growth, invasion, and metastasis.

The present disclosure relates to methods of treating or preventing cancer using CAR compositions of any preceding aspect.

In one aspect, disclosed herein are methods of treating, inhibiting, decreasing, reducing, ameliorating, and/or preventing a cancer and/or metastasis in a subject in need thereof, the method comprising administering a therapeutically effective amount of the cells disclosed herein. For example, disclosed herein are methods of treating, inhibiting, decreasing, reducing, ameliorating, and/or preventing a cancer and/or metastasis in a subject in need thereof, the method comprising administering a therapeutically effective amount of a cell comprising a bispecific chimeric antigen receptor (CAR) polypeptide, wherein the polypeptide comprises at least two tumor associated antigen (TAA) binding domains selected from the group consisting of an anti-CD33 binding domain, an anti-CLEC12A binding domain, an anti-CD123 binding domain, an anti-CD99 binding domain, or any combination thereof. In some aspects, the cell comprises a bispecific CAR comprising at least two tumor associated antigen (TAA) binding domains, wherein the TAA binding domain comprises an anti-CD33 binding domain and an anti-CLEC12A binding domain, an anti-CD33 binding domain and an anti-CD123 binding domain; an anti-CD33 binding domain and an anti-CD99 binding domain; an anti-CLEC12A binding domain and an anti-CD99 binding domain; an anti-CLEC12A binding domain and an anti-CD123 binding domain; or an anti-CD123 binding domain and an anti-CD99 binding domain.

Also disclosed herein are methods of treating, inhibiting, decreasing, reducing, ameliorating, and/or preventing a cancer and/or metastasis in a subject in need thereof wherein the bispecific CAR polypeptide comprises at least two TAA binding domains comrpising but not limited to any TAA of any preceding aspect, an anti-CD19 binding domain, an anti-TAG-72 binding domain, an anti-TIM3 binding domain, an anti-CD83 binding domain, an anti-B cell maturation antigen (BCMA) binding domain, an anti-CD22 binding domain, an anti-CD38 binding domain, an anti-NCAM1 binding domain, an anti-CD5 binding domain, an anti-CD70 binding domain, an anti-MET binding domain, an anti-L1CAM binding domain, an anti-CD44 binding domain, an anti-SLAMF7 binding domain, an anti-EGFR binding domain, an anti-EPHA2 binding domain, an anti-GPC3 binding domain, an anti-HER2 binding domain, an anti-mesothelin, an anti-PDCD1 binding domain, or any combinations thereof.

In some aspect, disclosed herein is a method of any preceding aspect comprising a bispecific CAR polypeptide of any preceding aspect encoded by the nucleic acids sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some aspects, disclosed herein, the bispecific CAR polypeptide of any preceding aspect is encoded by the nucleic acid sequence as set forth in SEQ ID NO: 2, SEQ ID NO: 3, SEQ ID NO: 5, or SEQ ID NO: 7.

In some aspects, disclosed herein are methods of treating, inhibiting, decreasing, reducing, ameliorating, and/or preventing a cancer and/or metastasis in a subject in need thereof, wherein the cell is selected from the group consisting of a αβT cell, γδT cell, a natural killer (NK) cell, a natural killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

In some embodiments, the CARs, the vector, or the cell of any preceding aspect can be administered to a subject either alone, or as a pharmaceutical composition in combination with diluents and/or with other immunomodulating agents including, but not limited to IL-1α, IL-2, IL-4, IL-6, IL-8, IL-10, IL-12, IL-15, IL-18, IL-21, IL-22, IFN-γ, TNF-α, TGF-β, LIF, cytotoxins (including, but not limited to perforin and/or granzyme), CCL2, CCL1, CCL19, CCL22, CXCL12, CCL17, MIP-1α, MCP-1, GRO/KC, and/or CXCR3.

In some embodiments, the method further comprises administering an immunomodulating agent. In some embodiments, the immunomodulating agent is a PD-1 antibody, a PD-L1 antibody, a CTLA-4 antibody, or any combination thereof.

In some embodiments, cancer is a neoplasm or a tumor that is not sensitive to immunotherapies, chemoradiation, or any other conventional cancer therapies. In some embodiments, the cancer is a sarcoma, lymphoma, leukemia, carcinoma, blastoma, or germ cell tumor. A representative, but non-limiting list of cancers that the disclosed compositions, nucleic acids, vectors, and cells of any preceding aspect can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of the head and neck, renal cancer, lung cancers such as small cell lung cancer and non-small lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, genitourinary cancer, pulmonary cancer, esophageal carcinoma, head and neck carcinoma, large bowl cancer, hematopoietic cancers, testicular cancer, colon cancer, and rectal cancer.

In some embodiments, the subject is a mammal. In some embodiments, the subject is a mouse, a rat, a dog, a cat, a hamster, a rabbit, or a non-human primate.

In some embodiments, the subject is a human.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, B1754111, Sym022, FS118, MGD013, and Immutep).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

SEQUENCES
1. anti-CD33-CLEC12A scFv1
SEQ ID NO. 1
CCATGGCCCTCCCGGTAACGGCTCTGCTGCTTCCACTCGCACTGCTCTTGCATGCTGCC
AGACCACAGGTCCAACTGCAGCAGCCTGGGGCTGAGCTGGTGAGGCCTGGAGTTTCAG
TGAAGCTGTCCTGCAAGGCTTCTGGCTACACCTTCACCAGCTACTGGATGAACTGGGTG
AAGCAGAGGCCTGGACAAGGCCTTGAGTGGATTGGCATGATTCATCCTTCCGATAGTG
AAACTAGGTTAAATCAGAAGTTCAAGGACAAGGCCATATTGACTGTAGACAAATCCTC
CAGCACAGCCTACATGCAACTCAGCAGCCCGACATCTGAGGACTCTGCGGTCTATTACT
GTGCAAGAGAAGAGGGACAGCTCGGGCACGGCGGTGCTATGGACTACTGGGGTCAAG
GAACCTCAGTCACCGTCTCCTCAGGTGGAGGTGGATCAGGTGGAGGTGGATCTGGTGG
AGGTGGATCTGATATCCAGATGACACAGACTACATCCTCCCTGTCTGCCTCTCTGGGAG
ACAGAGTCACCATCAGTTGCAGGGCAAGTCAGGACATTAGCAAGTATTTAAACTGGTA
TCAGCAGAAACCAGATGGAACTGTTAAACTCCTGATCTACTACACATCAAGATTACAC
TCAGGAGTCCCATCGAGGTTCAGTGGCAGTGGGTCTGGAACAGATTATTCTCTCACCAT
TAGCAACCTGGAGCAAGAAGATATTGCCACTTACTTTTGCCAACAGGGTGATACGTTTC
CGTGGACGTTCGGTGGAGGCACCAAGCTGGAAATCAAACGGGGTGGAGGTGGATCAG
GTGGAGGTGGATCTGGTGGAGGTGGATCTGAGCTGATTCTGGTGGAGTCCGGAGGCGG
ACTGGTGAAGCCCGGTGGCTCTTTAAAGCTGAGCTGCGCCGTGAGCGGCTTCACCTTCA
GCTCCTTCGCCATGAGCTGGGTTCGTCAGACCCCCGAAAAGAGGCTGGAATGGGTCGC
CACCATCTCCAGCGGCGGCGCCTACACCTTCTACAAGGACAGCGTGAAGGGTCGTTTC
ACAATCTCTCGTGACAACGCCAAGAACACCCTCTATTTACAGATGAGCAGCTTACGTA
GCGAGGACAGCGCCATGTACTACTGCGCCAGACACAGCGGATACGACGGCTACTACCT
CTATGCTATGGATTACTGGGGCCAAGGTACCTCCGTGACCGTGAGCTCTGGAGGAGGA
GGTAGCGGAGGAGGCGGAAGCGGTGGTGGAGGCTCCCAGATCGTGCTGACCCAGTCCC
CCGAGATTATGAGCGCCAGCCCCGGCGAGAAGGTGACCATGACTTGTTCCGCCTCCTC
CTCCGTGCATTACATGCACTGGTACCAGCAGAAGTCTGGCACCAGCCCCAAGAGGTGG
ATCTACGACACAAGCAAGCTGGCTAGCGGAGTGCCCGGAAGGTTCAGCGGGTCCGGCA
GCGGCACTAGCTACTCTTTAACCATCAGCAGCATGGAGAGCGAGGACGCTGCTACCTA
CTACTGTCAGCAGTGGACCTCCAATCCCCCTACCTTTGGCGGCGGCACCAAGCTGGAG
ATCAAGGCGGCCGCC
2. anti-CD33-CLEC12A scFv2
SEQ ID NO. 2
CCATGGCCCTCCCGGTAACGGCTCTGCTGCTTCCACTCGCACTGCTCTTGCATGCTGCC
AGACCAGAAGTGAAGCTGGTGGAGTCTGGGGGAGGCTTAGTGAAGCCTGGAGCGTCTC
TGAAACTCTCCTGTGCAGCCTCTGGATTCACTTTCAGTAACTATGGCATGTCTTGGGTTC
GCCAGACTTCAGACAAGAGGCTGGAGTGGGTCGCATCCATTAGTAGTGGTGGTGGTGA
CACCTACTATCCAGACAATGTAAAGGGCCGATTCACCATCTCCAGAGAGAATGCCAAG
AACACCCTGTACCTGCAAATGAGTAGTCTGAACTCTGAGGACACGGCCTTGTATTACTG
TGCAAGAGACTATGGTGGTACTTGGGACTACTTTGACTACTGGGGCCAAGGCACCACT
CTCACAGTCTCCTCAGGTGGAGGTGGATCAGGTGGAGGTGGATCTGGTGGAGGTGGAT
CTGATATCCAGATGACACAGACTACATCCTCCCTGTCTGCCTCTCTGGGAGACAGAGTC
ACCATCAGTTGCAGGGCAAGTCAGGACATTAGCAAGTATTTAAACTGGTATCAGCAGA
AACCAGATGGAACTGTTAAACTCCTGATCTACTACACATCAAGATTACACTCAGGAGT
CCCATCGAGGTTCAGTGGCAGTGGGTCTGGAACAGATTATTCTCTCACCATTAGCAACC
TGGAGCAAGAAGATATTGCCACTTACTTTTGCCAACAGGGTGATACGTTTCCGTGGACG
TTCGGTGGAGGCACCAAGCTGGAAATCAAACGGGGTGGAGGTGGATCAGGTGGAGGT
GGATCTGGTGGAGGTGGATCTGAGCTGATTCTGGTGGAGTCCGGAGGCGGACTGGTGA
AGCCCGGTGGCTCTTTAAAGCTGAGCTGCGCCGTGAGCGGCTTCACCTTCAGCTCCTTC
GCCATGAGCTGGGTTCGTCAGACCCCCGAAAAGAGGCTGGAATGGGTCGCCACCATCT
CCAGCGGCGGCGCCTACACCTTCTACAAGGACAGCGTGAAGGGTCGTTTCACAATCTC
TCGTGACAACGCCAAGAACACCCTCTATTTACAGATGAGCAGCTTACGTAGCGAGGAC
AGCGCCATGTACTACTGCGCCAGACACAGCGGATACGACGGCTACTACCTCTATGCTA
TGGATTACTGGGGCCAAGGTACCTCCGTGACCGTGAGCTCTGGAGGAGGAGGTAGCGG
AGGAGGCGGAAGCGGTGGTGGAGGCTCCCAGATCGTGCTGACCCAGTCCCCCGAGATT
ATGAGCGCCAGCCCCGGCGAGAAGGTGACCATGACTTGTTCCGCCTCCTCCTCCGTGCA
TTACATGCACTGGTACCAGCAGAAGTCTGGCACCAGCCCCAAGAGGTGGATCTACGAC
ACAAGCAAGCTGGCTAGCGGAGTGCCCGGAAGGTTCAGCGGGTCCGGCAGCGGCACT
AGCTACTCTTTAACCATCAGCAGCATGGAGAGCGAGGACGCTGCTACCTACTACTGTC
AGCAGTGGACCTCCAATCCCCCTACCTTTGGCGGCGGCACCAAGCTGGAGATCAAGGC
GGCCGCC
3. anti-CD33-CLEC12A scFv3
SEQ ID NO. 3
CCATGGCTTTACCCGTTACAGCTTTACTGCTGCCTCTGGCTTTATTATTACATGCCGCTA
GACCCGAGCTGATTCTGGTGGAGTCCGGAGGCGGACTGGTGAAGCCCGGTGGCTCTTT
AAAGCTGAGCTGCGCCGTGAGCGGCTTCACCTTCAGCTCCTTCGCCATGAGCTGGGTTC
GTCAGACCCCCGAAAAGAGGCTGGAATGGGTCGCCACCATCTCCAGCGGCGGCGCCTA
CACCTTCTACAAGGACAGCGTGAAGGGTCGTTTCACAATCTCTCGTGACAACGCCAAG
AACACCCTCTATTTACAGATGAGCAGCTTACGTAGCGAGGACAGCGCCATGTACTACT
GCGCCAGACACAGCGGATACGACGGCTACTACCTCTATGCTATGGATTACTGGGGCCA
AGGTACCTCCGTGACCGTGAGCTCTGGAGGAGGAGGTAGCGGAGGAGGCGGAAGCGG
TGGTGGAGGCTCCCAGATCGTGCTGACCCAGTCCCCCGAGATTATGAGCGCCAGCCCC
GGCGAGAAGGTGACCATGACTTGTTCCGCCTCCTCCTCCGTGCATTACATGCACTGGTA
CCAGCAGAAGTCTGGCACCAGCCCCAAGAGGTGGATCTACGACACAAGCAAGCTGGCT
AGCGGAGTGCCCGGAAGGTTCAGCGGGTCCGGCAGCGGCACTAGCTACTCTTTAACCA
TCAGCAGCATGGAGAGCGAGGACGCTGCTACCTACTACTGTCAGCAGTGGACCTCCAA
TCCCCCTACCTTTGGCGGCGGCACCAAGCTGGAGATCAAGGGTGGAGGTGGATCAGGT
GGAGGTGGATCTGGTGGAGGTGGATCTGAAGTGAAGCTGGTGGAGTCTGGGGGAGGCT
TAGTGAAGCCTGGAGCGTCTCTGAAACTCTCCTGTGCAGCCTCTGGATTCACTTTCAGT
AACTATGGCATGTCTTGGGTTCGCCAGACTTCAGACAAGAGGCTGGAGTGGGTCGCAT
CCATTAGTAGTGGTGGTGGTGACACCTACTATCCAGACAATGTAAAGGGCCGATTCAC
CATCTCCAGAGAGAATGCCAAGAACACCCTGTACCTGCAAATGAGTAGTCTGAACTCT
GAGGACACGGCCTTGTATTACTGTGCAAGAGACTATGGTGGTACTTGGGACTACTTTGA
CTACTGGGGCCAAGGCACCACTCTCACAGTCTCCTCAGGTGGAGGTGGATCAGGTGGA
GGTGGATCTGGTGGAGGTGGATCTGATATCCAGATGACACAGACTACATCCTCCCTGTC
TGCCTCTCTGGGAGACAGAGTCACCATCAGTTGCAGGGCAAGTCAGGACATTAGCAAG
TATTTAAACTGGTATCAGCAGAAACCAGATGGAACTGTTAAACTCCTGATCTACTACAC
ATCAAGATTACACTCAGGAGTCCCATCGAGGTTCAGTGGCAGTGGGTCTGGAACAGAT
TATTCTCTCACCATTAGCAACCTGGAGCAAGAAGATATTGCCACTTACTTTTGCCAACA
GGGTGATACGTTTCCGTGGACGTTCGGTGGAGGCACCAAGCTGGAAATCAAACGGGCG
GCCGCA
4. anti-CD33-CLEC12A scFv4
SEQ ID NO. 4
CCATGGCTTTACCCGTTACAGCTTTACTGCTGCCTCTGGCTTTATTATTACATGCCGCTA
GACCCGAGCTGATTCTGGTGGAGTCCGGAGGCGGACTGGTGAAGCCCGGTGGCTCTTT
AAAGCTGAGCTGCGCCGTGAGCGGCTTCACCTTCAGCTCCTTCGCCATGAGCTGGGTTC
GTCAGACCCCCGAAAAGAGGCTGGAATGGGTCGCCACCATCTCCAGCGGCGGCGCCTA
CACCTTCTACAAGGACAGCGTGAAGGGTCGTTTCACAATCTCTCGTGACAACGCCAAG
AACACCCTCTATTTACAGATGAGCAGCTTACGTAGCGAGGACAGCGCCATGTACTACT
GCGCCAGACACAGCGGATACGACGGCTACTACCTCTATGCTATGGATTACTGGGGCCA
AGGTACCTCCGTGACCGTGAGCTCTGGAGGAGGAGGTAGCGGAGGAGGCGGAAGCGG
TGGTGGAGGCTCCCAGATCGTGCTGACCCAGTCCCCCGAGATTATGAGCGCCAGCCCC
GGCGAGAAGGTGACCATGACTTGTTCCGCCTCCTCCTCCGTGCATTACATGCACTGGTA
CCAGCAGAAGTCTGGCACCAGCCCCAAGAGGTGGATCTACGACACAAGCAAGCTGGCT
AGCGGAGTGCCCGGAAGGTTCAGCGGGTCCGGCAGCGGCACTAGCTACTCTTTAACCA
TCAGCAGCATGGAGAGCGAGGACGCTGCTACCTACTACTGTCAGCAGTGGACCTCCAA
TCCCCCTACCTTTGGCGGCGGCACCAAGCTGGAGATCAAGGGTGGAGGTGGATCAGGT
GGAGGTGGATCTGGTGGAGGTGGATCTCAGGTCCAACTGCAGCAGCCTGGGGCTGAGC
TGGTGAGGCCTGGAGTTTCAGTGAAGCTGTCCTGCAAGGCTTCTGGCTACACCTTCACC
AGCTACTGGATGAACTGGGTGAAGCAGAGGCCTGGACAAGGCCTTGAGTGGATTGGCA
TGATTCATCCTTCCGATAGTGAAACTAGGTTAAATCAGAAGTTCAAGGACAAGGCCAT
ATTGACTGTAGACAAATCCTCCAGCACAGCCTACATGCAACTCAGCAGCCCGACATCT
GAGGACTCTGCGGTCTATTACTGTGCAAGAGAAGAGGGACAGCTCGGGCACGGCGGTG
CTATGGACTACTGGGGTCAAGGAACCTCAGTCACCGTCTCCTCAGGTGGAGGTGGATC
AGGTGGAGGTGGATCTGGTGGAGGTGGATCTGATATCCAGATGACACAGACTACATCC
TCCCTGTCTGCCTCTCTGGGAGACAGAGTCACCATCAGTTGCAGGGCAAGTCAGGACA
TTAGCAAGTATTTAAACTGGTATCAGCAGAAACCAGATGGAACTGTTAAACTCCTGAT
CTACTACACATCAAGATTACACTCAGGAGTCCCATCGAGGTTCAGTGGCAGTGGGTCT
GGAACAGATTATTCTCTCACCATTAGCAACCTGGAGCAAGAAGATATTGCCACTTACTT
TTGCCAACAGGGTGATACGTTTCCGTGGACGTTCGGTGGAGGCACCAAGCTGGAAATC
AAACGGGCGGCCGC
5. anti-CD33-CLEC12A scFv5
SEQ ID NO. 5
CCATGGCCCTCCCGGTAACGGCTCTGCTGCTTCCACTCGCACTGCTCTTGCATGCTGCC
AGACCAGAAGTGAAGCTGGTGGAGTCTGGGGGAGGCTTAGTGAAGCCTGGAGCGTCTC
TGAAACTCTCCTGTGCAGCCTCTGGATTCACTTTCAGTAACTATGGCATGTCTTGGGTTC
GCCAGACTTCAGACAAGAGGCTGGAGTGGGTCGCATCCATTAGTAGTGGTGGTGGTGA
CACCTACTATCCAGACAATGTAAAGGGCCGATTCACCATCTCCAGAGAGAATGCCAAG
AACACCCTGTACCTGCAAATGAGTAGTCTGAACTCTGAGGACACGGCCTTGTATTACTG
TGCAAGAGACTATGGTGGTACTTGGGACTACTTTGACTACTGGGGCCAAGGCACCACT
CTCACAGTCTCCTCAGGTGGAGGTGGATCTGAGCTGATTCTGGTGGAGTCCGGAGGCG
GACTGGTGAAGCCCGGTGGCTCTTTAAAGCTGAGCTGCGCCGTGAGCGGCTTCACCTTC
AGCTCCTTCGCCATGAGCTGGGTTCGTCAGACCCCCGAAAAGAGGCTGGAATGGGTCG
CCACCATCTCCAGCGGCGGCGCCTACACCTTCTACAAGGACAGCGTGAAGGGTCGTTT
CACAATCTCTCGTGACAACGCCAAGAACACCCTCTATTTACAGATGAGCAGCTTACGTA
GCGAGGACAGCGCCATGTACTACTGCGCCAGACACAGCGGATACGACGGCTACTACCT
CTATGCTATGGATTACTGGGGCCAAGGTACCTCCGTGACCGTGAGCTCTGGCAGCACC
AGCGGCAGCGGCAAACCGGGCAGCGGCGAAGGCAGCACCAAAGGCCAGATCGTGCTG
ACCCAGTCCCCCGAGATTATGAGCGCCAGCCCCGGCGAGAAGGTGACCATGACTTGTT
CCGCCTCCTCCTCCGTGCATTACATGCACTGGTACCAGCAGAAGTCTGGCACCAGCCCC
AAGAGGTGGATCTACGACACAAGCAAGCTGGCTAGCGGAGTGCCCGGAAGGTTCAGC
GGATCCGGCAGCGGCACTAGCTACTCTTTAACCATCAGCAGCATGGAGAGCGAGGACG
CTGCTACCTACTACTGTCAGCAGTGGACCTCCAATCCCCCTACCTTTGGCGGCGGCACC
AAGCTGGAGATCAAGGGTGGAGGTGGATCTGATATCCAGATGACACAGACTACATCCT
CCCTGTCTGCCTCTCTGGGAGACAGAGTCACCATCAGTTGCAGGGCAAGTCAGGACAT
TAGCAAGTATTTAAACTGGTATCAGCAGAAACCAGATGGAACTGTTAAACTCCTGATC
TACTACACATCAAGATTACACTCAGGAGTCCCATCGAGGTTCAGTGGCAGTGGGTCTG
GAACAGATTATTCTCTCACCATTAGCAACCTGGAGCAAGAAGATATTGCCACTTACTTT
TGCCAACAGGGTGATACGTTTCCGTGGACGTTCGGTGGAGGCACCAAGCTGGAAATCA
AACGGGCGGCCGCA
6. anti-CD33-CLEC12A scFv6
SEQ ID NO. 6
CCATGGCCCTCCCGGTAACGGCTCTGCTGCTTCCACTCGCACTGCTCTTGCATGCTGCC
AGACCACAGGTCCAACTGCAGCAGCCTGGGGCTGAGCTGGTGAGGCCTGGAGTTTCAG
TGAAGCTGTCCTGCAAGGCTTCTGGCTACACCTTCACCAGCTACTGGATGAACTGGGTG
AAGCAGAGGCCTGGACAAGGCCTTGAGTGGATTGGCATGATTCATCCTTCCGATAGTG
AAACTAGGTTAAATCAGAAGTTCAAGGACAAGGCCATATTGACTGTAGACAAATCCTC
CAGCACAGCCTACATGCAACTCAGCAGCCCGACATCTGAGGACTCTGCGGTCTATTACT
GTGCAAGAGAAGAGGGACAGCTCGGGCACGGCGGTGCTATGGACTACTGGGGTCAAG
GAACCTCAGTCACCGTCTCCTCAGGTGGAGGTGGATCTGAGCTGATTCTGGTGGAGTCC
GGAGGCGGACTGGTGAAGCCCGGTGGCTCTTTAAAGCTGAGCTGCGCCGTGAGCGGCT
TCACCTTCAGCTCCTTCGCCATGAGCTGGGTTCGTCAGACCCCCGAAAAGAGGCTGGA
ATGGGTCGCCACCATCTCCAGCGGCGGCGCCTACACCTTCTACAAGGACAGCGTGAAG
GGTCGTTTCACAATCTCTCGTGACAACGCCAAGAACACCCTCTATTTACAGATGAGCAG
CTTACGTAGCGAGGACAGCGCCATGTACTACTGCGCCAGACACAGCGGATACGACGGC
TACTACCTCTATGCTATGGATTACTGGGGCCAAGGTACCTCCGTGACCGTGAGCTCTGG
CAGCACCAGCGGCAGCGGCAAACCGGGCAGCGGCGAAGGCAGCACCAAAGGCCAGAT
CGTGCTGACCCAGTCCCCCGAGATTATGAGCGCCAGCCCCGGCGAGAAGGTGACCATG
ACTTGTTCCGCCTCCTCCTCCGTGCATTACATGCACTGGTACCAGCAGAAGTCTGGCAC
CAGCCCCAAGAGGTGGATCTACGACACAAGCAAGCTGGCTAGCGGAGTGCCCGGAAG
GTTCAGCGGATCCGGCAGCGGCACTAGCTACTCTTTAACCATCAGCAGCATGGAGAGC
GAGGACGCTGCTACCTACTACTGTCAGCAGTGGACCTCCAATCCCCCTACCTTTGGCGG
CGGCACCAAGCTGGAGATCAAGGGTGGAGGTGGATCTGATATCCAGATGACACAGACT
ACATCCTCCCTGTCTGCCTCTCTGGGAGACAGAGTCACCATCAGTTGCAGGGCAAGTCA
GGACATTAGCAAGTATTTAAACTGGTATCAGCAGAAACCAGATGGAACTGTTAAACTC
CTGATCTACTACACATCAAGATTACACTCAGGAGTCCCATCGAGGTTCAGTGGCAGTG
GGTCTGGAACAGATTATTCTCTCACCATTAGCAACCTGGAGCAAGAAGATATTGCCACT
TACTTTTGCCAACAGGGTGATACGTTTCCGTGGACGTTCGGTGGAGGCACCAAGCTGG
AAATCAAACGGGCGGCCGC
7. anti-CD33-CLEC12A scFv7
SEQ ID NO. 7
CCATGGCTTTACCCGTTACCGCTCTGCTGCTGCCTCTGGCTCTGCTGCTCCATGCTGCTC
GTCCCGAGCTGATTCTGGTGGAGTCCGGAGGCGGACTGGTGAAGCCCGGTGGCTCTTT
AAAGCTGAGCTGCGCCGTGAGCGGCTTCACCTTCAGCTCCTTCGCCATGAGCTGGGTTC
GTCAGACCCCCGAAAAGAGGCTGGAATGGGTCGCCACCATCTCCAGCGGCGGCGCCTA
CACCTTCTACAAGGACAGCGTGAAGGGTCGTTTCACAATCTCTCGTGACAACGCCAAG
AACACCCTCTATTTACAGATGAGCAGCTTACGTAGCGAGGACAGCGCCATGTACTACT
GCGCCAGACACAGCGGATACGACGGCTACTACCTCTATGCTATGGATTACTGGGGCCA
AGGTACCTCCGTGACCGTGAGCTCTGGTGGAGGTGGATCTGAAGTGAAGCTGGTGGAG
TCTGGGGGAGGCTTAGTGAAGCCTGGAGCGTCTCTGAAACTCTCCTGTGCAGCCTCTGG
ATTCACTTTCAGTAACTATGGCATGTCTTGGGTTCGCCAGACTTCAGACAAGAGGCTGG
AGTGGGTCGCATCCATTAGTAGTGGTGGTGGTGACACCTACTATCCAGACAATGTAAA
GGGCCGATTCACCATCTCCAGAGAGAATGCCAAGAACACCCTGTACCTGCAAATGAGT
AGTCTGAACTCTGAGGACACGGCCTTGTATTACTGTGCAAGAGACTATGGTGGTACTTG
GGACTACTTTGACTACTGGGGCCAAGGCACCACTCTCACAGTCTCCTCAGGCAGCACC
AGCGGCAGCGGCAAACCGGGCAGCGGCGAAGGCAGCACCAAAGGCGATATCCAGATG
ACACAGACTACATCCTCCCTGTCTGCCTCTCTGGGAGACAGAGTCACCATCAGTTGCAG
GGCAAGTCAGGACATTAGCAAGTATTTAAACTGGTATCAGCAGAAACCAGATGGAACT
GTTAAACTCCTGATCTACTACACATCAAGATTACACTCAGGAGTCCCATCGAGGTTCAG
TGGCAGTGGGTCTGGAACAGATTATTCTCTCACCATTAGCAACCTGGAGCAAGAAGAT
ATTGCCACTTACTTTTGCCAACAGGGTGATACGTTTCCGTGGACGTTCGGTGGAGGCAC
CAAGCTGGAAATCAAACGGGCGGGTGGAGGTGGATCTCAGATCGTGCTGACCCAGTCC
CCCGAGATTATGAGCGCCAGCCCCGGCGAGAAGGTGACCATGACTTGTTCCGCCTCCT
CCTCCGTGCATTACATGCACTGGTACCAGCAGAAGTCTGGCACCAGCCCCAAGAGGTG
GATCTACGACACAAGCAAGCTGGCTAGCGGAGTGCCCGGAAGGTTCAGCGGATCCGGC
AGCGGCACTAGCTACTCTTTAACCATCAGCAGCATGGAGAGCGAGGACGCTGCTACCT
ACTACTGTCAGCAGTGGACCTCCAATCCCCCTACCTTTGGCGGCGGCACCAAGCTGGA
GATCAAGCGGGCGGCCGCC
8. anti-CD33-CLEC12A scFv8
SEQ ID NO. 8
CCATGGCTTTACCCGTTACCGCTCTGCTGCTGCCTCTGGCTCTGCTGCTCCATGCTGCTC
GTCCCGAGCTGATTCTGGTGGAGTCCGGAGGCGGACTGGTGAAGCCCGGTGGCTCTTT
AAAGCTGAGCTGCGCCGTGAGCGGCTTCACCTTCAGCTCCTTCGCCATGAGCTGGGTTC
GTCAGACCCCCGAAAAGAGGCTGGAATGGGTCGCCACCATCTCCAGCGGCGGCGCCTA
CACCTTCTACAAGGACAGCGTGAAGGGTCGTTTCACAATCTCTCGTGACAACGCCAAG
AACACCCTCTATTTACAGATGAGCAGCTTACGTAGCGAGGACAGCGCCATGTACTACT
GCGCCAGACACAGCGGATACGACGGCTACTACCTCTATGCTATGGATTACTGGGGCCA
AGGTACCTCCGTGACCGTGAGCTCTGGTGGAGGTGGATCTCAGGTCCAACTGCAGCAG
CCTGGGGCTGAGCTGGTGAGGCCTGGAGTTTCAGTGAAGCTGTCCTGCAAGGCTTCTG
GCTACACCTTCACCAGCTACTGGATGAACTGGGTGAAGCAGAGGCCTGGACAAGGCCT
TGAGTGGATTGGCATGATTCATCCTTCCGATAGTGAAACTAGGTTAAATCAGAAGTTCA
AGGACAAGGCCATATTGACTGTAGACAAATCCTCCAGCACAGCCTACATGCAACTCAG
CAGCCCGACATCTGAGGACTCTGCGGTCTATTACTGTGCAAGAGAAGAGGGACAGCTC
GGGCACGGCGGTGCTATGGACTACTGGGGTCAAGGAACCTCAGTCACCGTCTCCTCAG
GCAGCACCAGCGGCAGCGGCAAACCGGGCAGCGGCGAAGGCAGCACCAAAGGCGATA
TCCAGATGACACAGACTACATCCTCCCTGTCTGCCTCTCTGGGAGACAGAGTCACCATC
AGTTGCAGGGCAAGTCAGGACATTAGCAAGTATTTAAACTGGTATCAGCAGAAACCAG
ATGGAACTGTTAAACTCCTGATCTACTACACATCAAGATTACACTCAGGAGTCCCATCG
AGGTTCAGTGGCAGTGGGTCTGGAACAGATTATTCTCTCACCATTAGCAACCTGGAGC
AAGAAGATATTGCCACTTACTTTTGCCAACAGGGTGATACGTTTCCGTGGACGTTCGGT
GGAGGCACCAAGCTGGAAATCAAACGGGGTGGAGGTGGATCTCAGATCGTGCTGACCC
AGTCCCCCGAGATTATGAGCGCCAGCCCCGGCGAGAAGGTGACCATGACTTGTTCCGC
CTCCTCCTCCGTGCATTACATGCACTGGTACCAGCAGAAGTCTGGCACCAGCCCCAAGA
GGTGGATCTACGACACAAGCAAGCTGGCTAGCGGAGTGCCCGGAAGGTTCAGCGGATC
CGGCAGCGGCACTAGCTACTCTTTAACCATCAGCAGCATGGAGAGCGAGGACGCTGCT
ACCTACTACTGTCAGCAGTGGACCTCCAATCCCCCTACCTTTGGCGGCGGCACCAAGCT
GGAGATCAAGCGGGCGGCCGCC
9. anti-CD123, 3F5HC1
SEQ ID NO. 9
EVQLQQSGPELVKPGSSVKISCKASGYTFTDYNMDWVKQSHGKSLEWIGTINPNN
GGTSYNQKFKGKATLTVDKSSSTAYMELRSLTSEDSAVYYCARKGYGGNYDYFDY
WGQGTTLTVSS
10. anti-CD123, 12H1HC1
SEQ ID NO. 10
EVQLQQSGAELVKPGASVKLSCTASGFNIKDTYMHWVKQRPEQGLEWIGRIDPAN
GNTIYASKFQGKATITADTSSNTAYMQLSSLTSGDTAVYYCALYYYGGSLDYWGQG
TTLTVSS
11. anti-CD 123, 12H1HC2
SEQ ID NO. 11
QVTLKESGPGILQPSQTLSLTCSFSGFSLSTYGMGVSWIRQPSGKGLEWLAHIYWD D D
KRYN PS LKSR LTI S KDTSN N Q VF LKI TS VDTA DTATYYCAQS LI YDG YYG FAYWG
QGTLVTVSA
12. anti-CD 123, 12H2HC1
SEQ ID NO. 12
QIQLVQSGPELKKPGETVKISCKASGYTFTYYGMNWVKQAPGKGLEWMGWINTYS
GVPTYADDFKGRFAFSLETSVSTAYLQINNLKNEDTATYFCARWIYYSDLYGMDYW
GQGTSVTVSS
13. anti-CD 123, 5A12HC1
SEQ ID NO. 13
QVQLQQSGAELAKPGASVKMSCKASGYTFSSYWMHWLKQRPGQGLEWIGYINPS
SGYTNYNQKFKDKATLTADKSSSTAYMQLSSLTSEDSAVYYCARDGNYDHWYFDV
WGTGTTVTVSS
14. anti-CD 123, 15A12HC2
SEQ ID NO. 14
QVQLQQPGAELVRPGASVKMSCKASGYTLTTYLMDWVKQRLGQGFEWIGNINPNS
GSSNYNEKFKGKAKLTVDKSSSTAYMQLSSLTSEDSAVYYCAIRHYGGSLFDYWG
QGTTLTVSS
15. anti-CD 123, 3F5LC1
SEQ ID NO. 15
DI LLTQS PA ILS VS PG E R VS FSC R ASQS I GTSI HWYQQRTNGSPRLLI
KYASESISGI P SRFSGSGSGTDFTLSINSVESEDIADYYCQQSNSWPYTFGGGTKLEIK
16. anti-CD123, 12H1LC1
SEQ ID NO. 16
DIVMSQSPSSLAVSVGERVTMSCKSSQSLLYSGNQKNYLAWYQQKPGQSPKLLIY
WASTRESGVPDRFTGSGSGTDFTLTISSVKAEDLAVYYCQQYYSYPRTFGGGTKLE IK
17. anti-CD 123, 12H2LC
SEQ ID NO. 17
DVLMTQSPLSLPVSLGDQASISCRSSQSIVHSNGDTYLEWYLQKPGQSPKLLIYKVS
NRFSGVPDRFSGSGSGTDFTLKISRVEAEDLGVYHCFQGSHVPWTFGGGTKLEIK
18. anti-CD 123, 15A12LC1
SEQ ID NO. 18
DIKMTQSPSSMYASLGERVTITCKASQDINSYLSWFQQKPGKSPKTLIYRANRLVDG
VPSRFSGSGSGQDYSLTISSLEYEDMGIYYCLQYDELLTFGAGTKLELK
19. anti-CD 123, 15A12LC2
SEQ ID NO. 19
DIVMSQSPSSLAVSAGERVTMSCRSSQSLLNSRTRKNYLAWYQQKPGQSPKLLIY
WASTRESGVPDRFSGSGSGTDFTLTISSVQAEDLAVYYCEQSYNLFTFGSGTKLEI
K
20. anti-CD 123, 3F5HC1_LC
SEQ ID NO. 20
EVQLQQSGPELVKPGSSVKISCKASGYTFTDYNMDWVKQSHGKSLEWIGTINPNN
GGTSYNQKFKGKATLTVDKSSSTAYMELRSLTSEDSAVYYCARKGYGGNYDYFDY
WGQGTTLTVSSGGGGSGGGGSGGGGSDILLTQSPAILSVSPGERVSFSCRASQSI
GTSI HWYQQRTNGSPRLLI KYASESISGI PSRFSGSGSGTDFTLSI
NSVESEDIADYY CQQSNSWPYTFGGGTKLEIK
21. anti-CD 123, 12H1HC1_LC1
SEQ ID NO. 21
EVQLQQSGAELVKPGASVKLSCTASGFNIKDTYMHWVKQRPEQGLEWIGRIDPAN
GNTIYASKFQGKATITADTSSNTAYMQLSSLTSGDTAVYYCALYYYGGSLDYWGQG
TTLTVSSGGGGSGGGGSGGGGSDIVMSQSPSSLAVSVGERVTMSCKSSQSLLYS
GNQKNYLAWYQQKPGQSPKLLIYWASTRESGVPDRFTGSGSGTDFTLTISSVKAE
DLAVYYCQQYYSYPRTFGGGTKLEIK
22. anti-CD 123, 12H1HC2_LC1
SEQ ID NO. 22
QVTLKESGPGILQPSQTLSLTCSFSGFSLSTYGMGVSWIRQPSGKGLEWLAHIYWD D D
KRYN PS LKSR LTI S KDTSN N Q VF LKI TS VDTA DTATYYCAQS LI YDG YYG
FAYWGQGTLVTVSAGGGGSGGGGSGGGGSDIVMSQSPSSLAVSVGERVTMSCKSSQSLLY
SGNQKNYLAWYQQKPGQSPKLLIYWASTRESGVPDRFTGSGSGTDFTLTISSVKA
EDLAVYYCQQYYSYPRTFGGGTKLEIK
23. anti-CD 123, 12H2HC1_LC1
SEQ ID NO. 23
QIQLVQSGPELKKPGETVKISCKASGYTFTYYGMNWVKQAPGKGLEWMGWINTYS
GVPTYADDFKGRFAFSLETSVSTAYLQINNLKNEDTATYFCARWIYYSDLYGMDYW
GQGTSVTVSSGGGGSGGGGSGGGGSDVLMTQSPLSLPVSLGDQASISCRSSQSI
VHSNGDTYLEWYLQKPGQSPKLLIYKVSNRFSGVPDRFSGSGSGTDFTLKISRVEA
EDLGVYHCFQGSHVPWTFGGGTKLEIK
24. anti-CD 123, 15A12HC1_LC1
SEQ ID NO. 24
QVQLQQSGAELAKPGASVKMSCKASGYTFSSYWMHWLKQRPGQGLEWIGYINPS
SGYTNYNQKFKDKATLTADKSSSTAYMQLSSLTSEDSAVYYCARDGNYDHWYFDV
WGTGTTVTVSSGGGGSGGGGSGGGGSDIKMTQSPSSMYASLGERVTITCKASQDI
NSYLSWFQQKPGKSPKTLIYRANRLVDGVPSRFSGSGSGQDYSLTISSLEYEDMGI
YYCLQYDELLTFGAGTKLELK
25. anti-CD 123, 15A12HC1_LC2
SEQ ID NO. 25
QVQLQQSGAELAKPGASVKMSCKASGYTFSSYWMHWLKQRPGQGLEWIGYINPS
SGYTNYNQKFKDKATLTADKSSSTAYMQLSSLTSEDSAVYYCARDGNYDHWYFDV
WGTGTTVTVSSGGGGSGGGGSGGGGSDIVMSQSPSSLAVSAGERVTMSCRSSQ
SLLNSRTRKNYLAWYQQKPGQSPKLLIYWASTRESGVPDRFSGSGSGTDFTLTISS
VQAEDLAVYYCEQSYNLFTFGSGTKLEIK
26. anti-CD 123, 15A12HC2_LC1
SEQ ID NO. 26
QVQLQQPGAELVRPGASVKMSCKASGYTLTTYLMDWVKQRLGQGFEWIGNINPNS
GSSNYNEKFKGKAKLTVDKSSSTAYMQLSSLTSEDSAVYYCAIRHYGGSLFDYWG
QGTTLTVSSGGGGSGGGGSGGGGSDIKMTQSPSSMYASLGERVTITCKASQDINS
YLSWFQQKPGKSPKTLIYRANRLVDGVPSRFSGSGSGQDYSLTISSLEYEDMGIYY
CLQYDELLTFGAGTKLELK
27. anti-CD 123, 15A12HC2_LC2
SEQ ID NO. 27
QVQLQQPGAELVRPGASVKMSCKASGYTLTTYLMDWVKQRLGQGFEWIGNINPNS
GSSNYNEKFKGKAKLTVDKSSSTAYMQLSSLTSEDSAVYYCAIRHYGGSLFDYWG
QGTTLTVSSGGGGSGGGGSGGGGSDIVMSQSPSSLAVSAGERVTMSCRSSQSLL
NSRTRKNYLAWYQQKPGQSPKLLIYWASTRESGVPDRFSGSGSGTDFTLTISSVQA
EDLAVYYCEQSYNLFTFGSGTKLEIK
28. anti-CD 123, 3F5HC1_LC
SEQ ID NO. 28
CCATGGCCCTCCCGGTAACGGCTCTGCTGCTTCCACTCGCACTGCTCTTGCAT
GCTGCCAGACCAGAGGTCCAGCTGCAACAGTCTGGACCTGAGCTGGTGAAGCC
TGGGTCTTCAGTGAAGATATCCTGCAAAGCTTCTGGATACACATTCACTGACTAC
AACATGGACTGGGTGAAGCAGAGTCATGGAAAGAGCCTTGAGTGGATTGGAAC
TATTAATCCTAACAATGGTGGTACTAGCTACAACCAGAAGTTCAAGGGCAAGGC
CACATTGACTGTAGACAAGTCCTCCAGCACAGCCTACATGGAGCTCCGCAGCCT
GACATCTGAAGACTCTGCAGTCTATTACTGTGCAAGAAAGGGCTATGGTGGTAA
CTACGACTACTTTGACTACTGGGGCCAAGGCACCACTCTCACAGTCTCCTCAGG
TGGAGGTGGATCAGGTGGAGGTGGATCTGGTGGAGGTGGATCTGACATCTTGC
TGACTCAGTCTCCAGCCATCCTGTCTGTGAGTCCAGGAGAAAGAGTCAGTTTCT
CCTGCAGGGCCAGTCAGAGCATTGGCACAAGCATACACTGGTATCAGCAAAGA
ACAAATGGTTCTCCAAGGCTTCTCATAAAGTATGCTTCTGAGTCTATCTCTGGGT
TCCCTTCCAGGTTTAGTGGCAGTGGATCAGGGACAGATTTTACTCTTAGCATCA
ACAGTGTGGAGTCTGAAGATATTGCAGATTATTACTGTCAACAAAGTAATAGCTG
GCCGTACACGTTCGGAGGGGGGACCAAGCTGGAAATAAAACGGGCGGCCGCA
29. anti-CD 123, 12H1HC1_LC1
SEQ ID NO. 29
ATGGCCCTCCCGGTAACGGCTCTGCTGCTTCCACTCGCACTGCTCTTGCATGCT
GCCAGACCAGAGGTTCAGCTGCAGCAGTCTGGGGCAGAGCTTGTGAAGCCAG
GGGCCTCAGTCAAGTTGTCCTGCACAGCTTCTGGCTTCAACATTAAAGACACCT
ATATGCACTGGGTGAAGCAGAGGCCTGAACAGGGCCTGGAGTGGATTGGAAGG
ATTGATCCTGCGAATGGTAATACTATATATGCCTCAAAGTTCCAGGGCAAGGCC
ACTATAACAGCAGACACATCATCCAACACAGCCTACATGCAGCTCAGCAGCCTG
ACATCTGGGGACACTGCCGTCTATTACTGTGCTCTTTATTACTATGGTGGTAGC
CTTGACTACTGGGGCCAAGGCACCACTCTCACAGTCTCCTCAGGTGGAGGTGG
ATCAGGTGGAGGTGGATCTGGTGGAGGTGGATCTGACATTGTGATGTCACAGT
CTCCATCCTCCCTAGCTGTGTCAGTTGGAGAGAGGGTTACTATGAGCTGCAAGT
CCAGTCAGAGCCTTTTATATAGTGGCAATCAAAAGAACTACTTGGCCTGGTACC
AGCAGAAACCAGGGCAGTCTCCTAAACTGCTGATTTACTGGGCATCCACTAGGG
AATCTGGGGTCCCTGATCGCTTCACAGGCAGTGGATCTGGGACAGATTTCACTC
TCACCATCAGCAGTGTGAAGGCTGAAGACCTGGCAGTTTATTACTGTCAGCAAT
ATTATAGCTATCCTCGGACGTTCGGTGGAGGCACCAAGCTGGAAATCAAACGG
30. anti-CD 123, 12H1HC1_LC2
SEQ ID NO. 30
ATGGCCCTCCCGGTAACGGCTCTGCTGCTTCCACTCGCACTGCTCTTGCATGCT
GCCAGACCACAGGTTACTCTGAAAGAGTCTGGCCCTGGGATATTGCAGCCCTC
CCAGACCCTCAGTCTGACTTGTTCTTTCTCTGGGTTTTCACTGAGCACTTATGGT
ATGGGTGTGAGCTGGATTCGTCAGCCTTCAGGAAAGGGTCTGGAGTGGCTGGC
ACACATTTACTGGGATGATGACAAGCGCTATAACCCATCCCTGAAGAGCCGGCT
CACAATCTCCAAGGATACCTCCAACAACCAGGTATTCCTCAAGATCACCAGTGT
GGACACTGCAGATACTGCCACATACTACTGTGCTCAAAGCCTGATCTATGATGG
TTACTACGGGTTTGCCTACTGGGGCCAAGGGACTCTGGTCACTGTCTCTGCAG
GTGGAGGTGGATCAGGTGGAGGTGGATCTGGTGGAGGTGGATCTGACATTGTG
ATGTCACAGTCTCCATCCTCCCTAGCTGTGTCAGTTGGAGAGAGGGTTACTATG
AGCTGCAAGTCCAGTCAGAGCCTTTTATATAGTGGCAATCAAAAGAACTACTTG
GCCTGGTACCAGCAGAAACCAGGGCAGTCTCCTAAACTGCTGATTTACTGGGC
ATCCACTAGGGAATCTGGGGTCCCTGATCGCTTCACAGGCAGTGGATCTGGGA
CAGATTTCACTCTCACCATCAGCAGTGTGAAGGCTGAAGACCTGGCAGTTTATT
ACTGTCAGCAATATTATAGCTATCCTCGGACGTTCGGTGGAGGCACCAAGCTGG
AAATCAAACGG
31. anti-CD 123, 12H2HC1_LC1
SEQ ID NO. 31
ATGGCCCTCCCGGTAACGGCTCTGCTGCTTCCACTCGCACTGCTCTTGCATGCT
GCCAGACCACAGATCCAGTTGGTGCAATCTGGACCTGAGCTGAAGAAGCCTGG
AGAGACAGTCAAGATCTCCTGCAAGGCTTCTGGGTATACCTTCACATACTATGG
AATGAACTGGGTGAAGCAGGCTCCAGGAAAGGGTTTAGAGTGGATGGGCTGGA
TAAACACCTACTCTGGAGTGCCAACATATGCTGATGACTTCAAGGGACGGTTTG
CCTTCTCTTTGGAAACCTCTGTCAGCACTGCCTATTTGCAGATCAACAACCTCAA
AAATGAGGACACGGCTACATATTTTTGTGCAAGATGGATCTACTATAGTGACCTC
TATGGTATGGACTACTGGGGTCAAGGAACCTCAGTCACCGTCTCCTCAGGTGG
AGGTGGATCAGGTGGAGGTGGATCTGGTGGAGGTGGATCTGATGTTTTGATGA
CCCAAAGTCCACTCTCCCTGCCTGTCAGTCTTGGAGATCAAGCCTCCATCTCTT
GTAGATCTAGTCAGAGTATTGTACATAGTAATGGAGACACGTATTTAGAATGGTATTTG
CAGAAACCAGGCCAGTCTCCAAAGCTCCTGATCTACAAAGTTTCTAACCG
ATTTTCTGGGGTCCCAGACAGGTTCAGTGGCAGTGGATCAGGGACAGATTTCAC
ACTCAAGATCAGCAGAGTGGAGGCTGAGGATCTGGGAGTTTATCACTGCTTTCA
AGGTTCACATGTTCCGTGGACGTTCGGTGGAGGCACCAAGCTGGAAATCAAAC GG
32. anti-CD 123, 15A12HC1_LC1
SEQ ID NO. 32
ATGGCCCTCCCGGTAACGGCTCTGCTGCTTCCACTCGCACTGCTCTTGCATGCT
GCCAGACCACAGGTCCAGCTGCAGCAGTCTGGGGCTGAACTGGCAAAACCTGG
GGCCTCAGTGAAGATGTCCTGCAAGGCTTCTGGCTACACCTTTTCTAGCTACTG
GATGCACTGGCTAAAACAGAGGCCTGGACAGGGTCTGGAGTGGATTGGATACA
TTAATCCTAGCAGTGGTTATACTAACTACAATCAGAAGTTCAAGGACAAGGCCACATT
GACTGCAGACAAATCCTCCAGCACAGCCTACATGCAACTGAGCAGCCTGACATCTGAG
GACTCTGCAGTCTATTACTGTGCAAGAGATGGTAACTATGACCACTG
GTACTTCGATGTCTGGGGCACAGGGACCACGGTCACCGTCTCCTCAGGTGGAG
GTGGATCAGGTGGAGGTGGATCTGGTGGAGGTGGATCTGACATCAAGATGACC
CAGTCTCCATCTTCCATGTATGCATCTCTAGGAGAGAGAGTCACTATCACTTGCA
AGGCGAGTCAGGACATTAATAGCTATTTAAGCTGGTTCCAGCAGAAACCAGGGA
AATCTCCTAAGACCCTGATCTATCGTGCAAACAGATTGGTAGATGGGGTCCCAT
CAAGGTTCAGTGGCAGTGGATCTGGGCAAGATTATTCTCTCACCATCAGCAGCC
TGGAGTATGAAGATATGGGAATTTATTATTGTCTACAGTATGATGAGTTGCTCAC
GTTCGGTGCTGGGACCAAGCTGGAGCTGAAACGG
33. anti-CD 123, 15A12HC1_LC2
SEQ ID NO. 33
ATGGCCCTCCCGGTAACGGCTCTGCTGCTTCCACTCGCACTGCTCTTGCATGCT
GCCAGACCACAGGTCCAGCTGCAGCAGTCTGGGGCTGAACTGGCAAAACCTGG
GGCCTCAGTGAAGATGTCCTGCAAGGCTTCTGGCTACACCTTTTCTAGCTACTG
GATGCACTGGCTAAAACAGAGGCCTGGACAGGGTCTGGAGTGGATTGGATACA
TTAATCCTAGCAGTGGTTATACTAACTACAATCAGAAGTTCAAGGACAAGGCCACATT
GACTGCAGACAAATCCTCCAGCACAGCCTACATGCAACTGAGCAGCCTGACATCTGAG
GACTCTGCAGTCTATTACTGTGCAAGAGATGGTAACTATGACCACTG
GTACTTCGATGTCTGGGGCACAGGGACCACGGTCACCGTCTCCTCAGGTGGAG
GTGGATCAGGTGGAGGTGGATCTGGTGGAGGTGGATCTGACATTGTGATGTCA
CAGTCTCCATCCTCCCTGGCTGTGTCAGCAGGAGAGAGGGTCACTATGAGCTG
CAGATCCAGTCAGAGTCTGCTCAACAGTAGAACCCGAAAGAACTACTTGGCTTG
GTACCAGCAGAAACCAGGGCAGTCTCCTAAGCTGCTGATCTACTGGGCATCCA
CTAGGGAATCTGGGGTCCCTGATCGCTTCTCAGGCAGTGGATCTGGGACAGAT
TTCACTCTCACCATCAGCAGTGTGCAGGCTGAAGACCTGGCAGTTTATTACTGC
GAGCAATCTTATAATCTATTCACGTTCGGCTCGGGGACAAAGTTGGAAATAAAA CGG
34. anti-CD 123, 15A12HC2_LC1
SEQ ID NO. 34
ATGGCCCTCCCGGTAACGGCTCTGCTGCTTCCACTCGCACTGCTCTTGCATGCT
GCCAGACCACAGGTTCAACTGCAGCAGCCTGGGGCTGAGCTGGTGAGGCCTG
GGGCTTCAGTGAAGATGTCCTGCAAGGCTTCTGGCTACACCCTCACCACCTACT
TGATGGACTGGGTAAAACAGAGGCTTGGACAAGGCTTTGAGTGGATTGGAAATA
TTAATCCTAATAGTGGTAGTAGTAACTACAATGAGAAGTTCAAGGGCAAGGCCA
AGCTGACTGTAGACAAATCCTCCAGCACAGCCTACATGCAACTCAGCAGCCTGA
CATCTGAGGACTCTGCGGTCTATTACTGTGCAATACGGCACTATGGTGGTAGTC
TCTTTGACTACTGGGGCCAAGGCACCACTCTCACAGTCTCCTCAGGTGGAGGT
GGATCAGGTGGAGGTGGATCTGGTGGAGGTGGATCTGACATCAAGATGACCCA
GTCTCCATCTTCCATGTATGCATCTCTAGGAGAGAGAGTCACTATCACTTGCAA
GGCGAGTCAGGACATTAATAGCTATTTAAGCTGGTTCCAGCAGAAACCAGGGAA
ATCTCCTAAGACCCTGATCTATCGTGCAAACAGATTGGTAGATGGGGTCCCATC
AAGGTTCAGTGGCAGTGGATCTGGGCAAGATTATTCTCTCACCATCAGCAGCCT
GGAGTATGAAGATATGGGAATTTATTATTGTCTACAGTATGATGAGTTGCTCACG
TTCGGTGCTGGGACCAAGCTGGAGCTGAAACGG
35. anti-CD 123, 15A12HC2_LC2
SEQ ID NO. 35
ATGGCCCTCCCGGTAACGGCTCTGCTGCTTCCACTCGCACTGCTCTTGCATGCT
GCCAGACCACAGGTTCAACTGCAGCAGCCTGGGGCTGAGCTGGTGAGGCCTG
GGGCTTCAGTGAAGATGTCCTGCAAGGCTTCTGGCTACACCCTCACCACCTACT
TGATGGACTGGGTAAAACAGAGGCTTGGACAAGGCTTTGAGTGGATTGGAAATA
TTAATCCTAATAGTGGTAGTAGTAACTACAATGAGAAGTTCAAGGGCAAGGCCA
AGCTGACTGTAGACAAATCCTCCAGCACAGCCTACATGCAACTCAGCAGCCTGA
CATCTGAGGACTCTGCGGTCTATTACTGTGCAATACGGCACTATGGTGGTAGTC
TCTTTGACTACTGGGGCCAAGGCACCACTCTCACAGTCTCCTCAGGTGGAGGT
GGATCAGGTGGAGGTGGATCTGGTGGAGGTGGATCTGACATTGTGATGTCACA
GTCTCCATCCTCCCTGGCTGTGTCAGCAGGAGAGAGGGTCACTATGAGCTGCA
GATCCAGTCAGAGTCTGCTCAACAGTAGAACCCGAAAGAACTACTTGGCTTGGT
ACCAGCAGAAACCAGGGCAGTCTCCTAAGCTGCTGATCTACTGGGCATCCACTA
GGGAATCTGGGGTCCCTGATCGCTTCTCAGGCAGTGGATCTGGGACAGATTTC
ACTCTCACCATCAGCAGTGTGCAGGCTGAAGACCTGGCAGTTTATTACTGCGAG
CAATCTTATAATCTATTCACGTTCGGCTCGGGGACAAAGTTGGAAATAAAACGG
36. anti-CD99, 1 H3H7, 1 H3H9
SEQ ID NO. 36
EVQLQQSGAELVKPGASVKLSCTASGFDIKDTYIHVWKQRPEQGLEWIGRIDPANGDTRYD
PEFQGKASLTADTSSNTAYLQFSNLTSEDTAVYYCARRGGLSWGQGTTLTVSS
37. anti-CD 99, 1 H3H7, 1 H3H9
SEQ ID NO. 37
GAGGTTCAACTGCAACAGTCTGGGGCAGAGCTTGTGAAGCCAGGGGCCTCAGTCA
AGTTGT CCTGCACAGCTT CT GGCTT CGACATT AAAGACACCT AT AT
CCACTGGGTGAAACAGAGGCCTGAACAGGGCCTGGAGTGGATTGGAAGGATTGATCCT
GCGAATG GTG AT ACT AG AT AT G ACCCGG AATT CCAG GG CAAG G CCTCT CT
AACAG CT G ACAC ATCCT CCAAT ACAGCCT ACCT ACAATT CAGCAACCT G ACAT CT
G AAG ACACT G CCGT CT ATT ACT GTG CT AG AAG AG GCG G CCT CT CCT G GG G
CCAAG G CACCACT CT CACA GTCTCCTCA
38. anti-CD99, 4C5E2
SEQ ID NO. 38
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSS
39. anti-CD 99, 4C5E2
SEQ ID NO. 39
GAGGTGCAGCTGGAGGAGTCTGGTGGAGGATTGGTGCAGCCTAAAGGATCATTGA
AACT CT CAT GT GCCGCCT CCGGTTT CACCTT CAAT ACCT AT GCCAT GT
ACTGGGTCTGCCAGGCTCCAGGAAAGGGTTTGAAATGGGTTGCTCGCATAAGAAGTAA
AGTTAAT AATT ATGCAACAT ATT AT GCCGATT CAGT GAAAG ACAG ATT CACCAT
CT CCAGAG AT GATT C ACAAAACAT G CT CTTT CT G CACAT G AACAACCT G
AAAACT GAGG ACACT G CC AT AT ATTT CTGTGT G AG AGAT CCT ATGG ACT ACT
GGGGT CAAGG AAT CT CAGT CACC GTCTCCTCA
40. anti-CD99, 4C5H10
SEQ ID NO. 40
EVQLVESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSS
41. anti-CD99, 4C5H10
SEQ ID NO. 41
GAGGT GCAGCTT GTT GAGT CTGGT GGAGGATT GGT GCAGCCTAAAGGAT
CATTGAAACTCTCATGTGCCGCCTCCGGTTTCACCTTCAATACCTATGCCATGTACTGG
GTCTGCCAGGCTCCAGGAAAGGGTTTGAAATGGGTTGCTCGCATAAGAAGTAAAGTTA
AT AATT ATGCAACAT ATT AT GCCGATT CAGT GAAAG ACAG ATT CACCAT CT
CCAGAG AT GATT C ACAAAACAT G CT CTTT CT G CACAT G AACAACCT G AAAACT
GAGG ACACT G CC AT AT ATTT CTGTGT GAG AGAT CCT ATGG ACT ACT GGGGT
CAAGG AAT CT CAGT CACC GTCTCCTCA
42. anti-CD99, 9G12C9
SEQ ID NO. 42
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQ
GTTLTVSS
43. anti-CD99, 9G12C9
SEQ ID NO. 43
CAGGTTCAGCTCCAGCAGTCTGGGGCTGAGCTGGCAAGACCTGGGGCTTCAGTGAAGT
TGTCCTGCAAGGCTTCTGGCTACACGTTTACTACTTTCTGGATGCAGTGGGTAAAACAG
AGGCCTGGACAGGGTCTGGAATGGATTGGGACTATTTATCCTGGAGATGA
TGATACTAGGTACACT C AG AAATT C AAG G G C AG G G C C AC ATT G ACT G C AG
AT AAAT CGT CCACCACAGCCT ACAT GCAACT CAGCAACTT GT
CATCTGAGGACTCTGCGGTCTATTACTGTGCAAGATCGGGGTATGAGAGGGGCCCATA
CTACTTTGACTCCTGGGG CCAAG G CACCACT CT CACAGT CT CCT CA
44. anti-CD99, 9G12G6 HB1
SEQ ID NO. 44
DVKLQESGAELARPGASVKLSCKASGYTFTTFWMQRVKQRPGQGLEWIGTIYPGDDDTRY
TQKFKGRATLTADKSSTTAYMQLSNLSSEDSAVYYCARSGYERGPYYFDSWGQG
TTLTVSS
45. anti-CD99, 9G12G6 HB1
SEQ ID NO. 45
GATGTGAAGCTTCAGGAGTCTGGGGCTGAGCTGGCAAGACCTGGGGCTTCAGTGAAGT
TGTCCTGCAAGGCTTCTGGCTACACGTTTACTACTTTCTGGATGCAGCGGGTAAAACAG
AGGCCTGGACAGGGTCTGGAATGGATTGGGACTATTTATCCTGGAGATGA
TGATACTAGGTACACT C AG AAATT C AAG G G C AG G G C C AC ATT G ACT G C AG
AT AAAT CGT CCACCACAGCCTACATGCAACT CAGCAACTT GT
CATCTGAGGACTCTGCGGTCTATTACTGTGCAAGATCGGGGTATGAGAGGGGCCCATA
CTACTTTGACTCCTGGGG CCAAG G CACCACT CT CACAGT CT CCT CA
46. anti-CD99, 9G12G6 HB3
SEQ ID NO. 46
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQG
TTLTVSS
47. anti-CD99, 9G12G6 HB3
SEQ ID NO. 47
CAGGTGCAGCTGAAGGAGTCTGGGGCTGAGCTGGCAAGACCTGGGGCTTCAGTGAAGT
TGTCCTGCAAGGCTTCTGGCTACACGTTTACTACTTTCTGGATGCAGTGGGCAAAACAG
AGGCCTGGACAGGGTCTGGAATGGATTGGGACTATTTATCCTGGAGATGATGATACTA
GGTACACTCAGAAATTCAAGGGCAGGGCCACATTGACTGCAGATAAATCGTCCACCAC
AGCCTACATGCAACTCAGCAACTTGTCATCTGAGGACTCTGCGGTCTATTACTGTGCAA
GATCGGGGTATGAGAGGGGCCCATACTACTTTGACTCCTGGGGCCAAGGCACCACTCT
CACAGTCTCCTCA
48. anti-CD99, 1 H3H9
SEQ ID NO. 48
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIK
49. anti-CD99, 1 H3H9
SEQ ID NO. 49
GACATCCAGATGACTCAGTCTCCAGCCTCCCTATCTGCATCTGTGGGAGAAACTGTCAC
CATCACATGTCGAGCAAGTGGGAATATTCACAATTATTTAGCATGGTATCAGCAGAAA
CAGGGAAAATCTCCTCAGCTCCTGGTCTATAATGCAAAAACCTTAGCAGATGGTGTGC
CATCAAGGTTCAGTGGCAGTGGATCAGGAACACAATATTCTCTCAAGATCAACAGCCT
GCAGCCTGAAGATTTTGGGAGTTATTACTGTCAACATTTTTGGAGTACTCCGTGGACGT
TCGGTGGAGGCACCAAGCTGGAAATCAAA
50. anti-CD99, 1 H3H7 LC1
SEQ ID NO. 50
GNSWSHSLRSLSVTIGQPASISCKSSQSLLDGNGKTYLNWLLQRPGQSPKRLLYLVSK
LDSGVPDRFTGSGSGTDFTLKISRVEAEDLGIYYCWQGTHFPRTFGGGTKLEIK
51. anti-CD99, 1 H3H7 LC2
SEQ ID NO. 51
GNSWRHSPRSLSVTIGQPASISCKSSQSLLDGNGKTYLNWLLQRPGQSPKRLLYLVSK
LDSGVPDRFTGSGSGTDFTLKISRVEAEDLGIYYCWQGTHFPRTFGGGTKLEIK
52. anti-CD99, 4C5E2
SEQ ID NO. 52
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIK
53. anti-CD99, 4C5E2
SEQ ID NO. 53
GATATTGTGATGACTCAGGCTGCACCCTCTGTACCTGTCACTCCTGGGGAGTCAGTATC
CATCTCCTGCGGGTCTAGTAAGAGTCTCCTGCATAGTAATGGCAACACTTACTTGTATT
GGTTCCTGCAGAGGCCAGGCCAGTCTCCTCAGCTCCTGATATATCGGGTGTCCAACCTT
GCCTCAGGAGTCCCAGACAGGTTCAGTGGCAGTGGGTCAGGAACTGCTTTCACACTGA
GAATCAGTAGAGTGGAGGCTGAGGATGTGGGTGTTTATTACTGTATGCAACATCTAGA
ATATCCGTACACGTTCGGAGGGGGGACCAGGCTGGAAATAAAA
54. anti-CD99, 9G12C9
SEQ ID NO. 54
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELK
55. anti-CD99, 9G12C9
SEQ ID NO. 55
GACACTGTGATGTCACAGTCCCCATCCTCCCTAGCTGTTTCAGTTGGAGAGAAGATAAC
TATGAGCTGCAAGTCCAGTCAGAGTCTTTTATGTCGTAGCAATCAAAAGAACTACTTGG
CCTGGTACCAGCAGAAACCAGGGCAGTCTCCTAAACAGCTGATTTACTGGGCATCTAC
TAGGGAATCTGGGGTCCCTGATCGCTTCACAGGCAGTGGATCTGGGACAGATTTCACTC
TCACCATCAGCAGTGTGAAGGCTGAAGACCTGGCAGTTTATTACTGTCAGCAATATTAT
AGTTATCCGCTCACGTTCGGTGCTGGCACCAAGCTGGAGCTGAAA
56. anti-CD99, 9G12G6
SEQ ID NO. 56
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELK
57. anti-CD99, 9G12G6
SEQ ID NO. 57
GACACTGTGATGTCACAGTCCCCATCCTCCCTAGCTGTTTCAGTTGGAGAGAAGATAAC
TATGAGCTGCAAGTCCAGTCAGAGTCTTTTATATCGTAGCAATCAAAAGAACTACTTGG
CCTGGTACCAGCAGAAACCAGGGCAGTCTCCTAAACAGCTGATTTACTGGGCATCTAC
TAGGGAATCTGGGGTCCCTGATCGCTTCACAGGCAGTGGATCTGGGACAGATTTCACTC
TCACCATCAGCAGTGTGAAGGCTGAAGACCTGGCAGTTTATTACTGTCAGCAATATTAT
AGTTATCCGCTCACGTTCGGTGCTGGCACCAAGCTGGAGCTGAAA
58. anti-CD99, 1 H3H9 v1
SEQ ID NO. 58
EVQLQQSGAELVKPGASVKLSCTASGFDIKDTYIHVWKQRPEQGLEWIGRIDPANGDTRYD
PEFQGKASLTADTSSNTAYLQFSNLTSEDTAVYYCARRGGLSWGQGTTLTVSSGGGGSGG
GGSGGGGSDIQMTQSPASLSASVGETVTITCRASGNIHNYLAWYQQKQGKSPQLLVYNAK
TLADGVPSRFSGSGSGTQYSLKINSLQPEDFGSYYCQHFWSTPWTFGGGTKLEIK
59. anti-CD99 scFv
SEQ ID NO. 59
EVQLQQSGAELVKPGASVKLSCTASGFDIKDTYIHVWKQRPEQGLEWIGRIDPANGDTRYD
PEFQGKASLTADTSSNTAYLQFSNLTSEDTAVYYCARRGGLSWGQGTTLTVSSGGGGSGG
GGSGGGGSGNSWSHSLRSLSVTIGQPASISCKSSQSLLDGNGKTYLNWLLQRPGQSPKRLL
YLVSKLDSGVPDRFTGSGSGTDFTLKISRVEAEDLGIYYCWQGTHFPRTFGGGTKLEIK
60. anti-CD99 scFv
SEQ ID NO. 60
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDIQMTQSPASLSASVGETVTITCRASGNIHNYLAWYQQKQGKSPQLLV
YNAKTLADGVPSRFSGSGSGTQYSLKINSLQPEDFGSYYCQHFWSTPWTFGGGTKLEIK
61. anti-CD99 scFv
SEQ ID NO. 61
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSGNSWSHSLRSLSVTIGQPASISCKSSQSLLDGNGKTYLNWLLQRPGQSP
KRLLYLVSKLDSGVPDRFTGSGSGTDFTLKISRVEAEDLGIYYCWQGTHFPRTFGGGTKLEI
K
62. anti-CD99, 4C5E2 v1
SEQ ID NO. 62
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLEYPYTFGGGTRLEI
K
63. anti-CD99 scFv
SEQ ID NO. 63
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDTVMSQSPSSLAVSVGEKITMSCKSSQSLLCRSNQKNYLAWYQQKPGQ
SPKQLIYWASTRESGVPDRFTGSGSGTDFTLTISSVKAEDLAVYYCQQYYSYPLTFGAGTKL
ELK
64. anti-CD99 scFv
SEQ ID NO. 64
EVQLEESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDTVMSQSPSSLAVSVGEKITMSCKSSQSLLYRSNQKNYLAWYQQKPGQ
SPKQLIYWASTRESGVPDRFTGSGSGTDFTLTISSVKAEDLAVYYCQQYYSYPLTFGAGTKL
ELK
65. anti-CD99 scFv
SEQ ID NO. 65
EVQLVESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDIQMTQSPASLSASVGETVTITCRASGNIHNYLAWYQQKQGKSPQLLV
YNAKTLADGVPSRFSGSGSGTQYSLKINSLQPEDFGSYYCQHFWSTPWTFGGGTKLEIK
66. anti-CD99 scFv
SEQ ID NO. 66
EVQLVESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSGNSWSHSLRSLSVTIGQPASISCKSSQSLLDGNGKTYLNWLLQRPGQSP
KRLLYLVSKLDSGVPDRFTGSGSGTDFTLKISRVEAEDLGIYYCWQGTHFPRTFGGGTKLEI
K
67. anti-CD99, 4C5H10 v1)
SEQ ID NO. 67
EVQLVESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLEYPYTFGGGTRLEI
K
68. anti-CD99 scFv
SEQ ID NO. 68
EVQLVESGGGLVQPKGSLKLSCAASGFTFNTYAMYVWCQAPGKGLKVWARIRSKVNNYA
TYYADSVKDRFTISRDDSQNMLFLHMNNLKTEDTAIYFCVRDPMDYWGQGISVTVSSGGG
GSGGGGSGGGGSDTVMSQSPSSLAVSVGEKITMSCKSSQSLLCRSNQKNYLAWYQQKPGQ
SPKQLIYWASTRESGVPDRFTGSGSGTDFTLTISSVKAEDLAVYYCQQYYSYPLTFGAGTKL
ELK
69. anti-CD99 scFv
SEQ ID NO. 69
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDIQMTQSPASLSASVGETVTITCRASGNIHNYLAWYQQKQGKS
PQLLVYNAKTLADGVPSRFSGSGSGTQYSLKINSLQPEDFGSYYCQHFWSTPWTFGGGTKL
EIK
70. anti-CD99 scFv
SEQ ID NO. 70
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSGNSWSHSLRSLSVTIGQPASISCKSSQSLLDGNGKTYLNWLLQR
PGQSPKRLLYLVSKLDSGVPDRFTGSGSGTDFTLKISRVEAEDLGIYYCWQGTHFPRTFGGG
TKLEIK
71. anti-CD99 scFv
SEQ ID NO. 71
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDIVMTQAAPSVPVTPGESVSISCGSSKSLLHSNGNTYLYWFLQR
PGQSPQLLIYRVSNLASGVPDRFSGSGSGTAFTLRISRVEAEDVGVYYCMQHLEYPYTFGG
GTRLEIK
72. anti-CD99, 9G12C9 v1
SEQ ID NO. 72
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDTVMSQSPSSLAVSVGEKITMSCKSSQSLLCRSNQKNYLAWYQ
QKPGQSPKQLIYWASTRESGVPDRFTGSGSGTDFTLTISSVKAEDLAVYYCQQYYSYPLTF
GAGTKLELK
73. anti-CD99 scFv
SEQ ID NO. 73
QVQLQQSGAELARPGASVKLSCKASGYTFTTFWMQWVKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDTVMSQSPSSLAVSVGEKITMSCKSSQSLLYRSNQKNYLAWYQ
QKPGQSPKQLIYWASTRESGVPDRFTGSGSGTDFTLTISSVKAEDLAVYYCQQYYSYPLTF
GAGTKLELK
74. anti-CD99 scFv
SEQ ID NO. 74
DVKLQESGAELARPGASVKLSCKASGYTFTTFWMQRVKQRPGQGLEWIGTIYPGDDDTRY
TQKFKGRATLTADKSSTTAYMQLSNLSSEDSAVYYCARSGYERGPYYFDSWGQGTTLTVS
SGGGGSGGGGSGGGGSDIQMTQSPASLSASVGETVTITCRASGNIHNYLAWYQQKQGKSP
QLLVYNAKTLADGVPSRFSGSGSGTQYSLKINSLQPEDFGSYYCQHFWSTPWTFGGGTKLE
IK
75. anti-CD99 scFv
SEQ ID NO. 75
DVKLQESGAELARPGASVKLSCKASGYTFTTFWMQRVKQRPGQGLEWIGTIYPGDDDTRY
TQKFKGRATLTADKSSTTAYMQLSNLSSEDSAVYYCARSGYERGPYYFDSWGQGTTLTVS
SGGGGSGGGGSGGGGSGNSWSHSLRSLSVTIGQPASISCKSSQSLLDGNGKTYLNWLLQRP
GQSPKRLLYLVSKLDSGVPDRFTGSGSGTDFTLKISRVEAEDLGIYYCWQGTHFPRTFGGG
TKLEIK
76. anti-CD99 scFv
SEQ ID NO. 76
DVKLQESGAELARPGASVKLSCKASGYTFTTFWMQRVKQRPGQGLEWIGTIYPGDDDTRY
TQKFKGRATLTADKSSTTAYMQLSNLSSEDSAVYYCARSGYERGPYYFDSWGQGTTLTVS
SGGGGSGGGGSGGGGSDIVMTQAAPSVPVTPGESVSISCGSSKSLLHSNGNTYLYWFLQRP
GQSPQLLIYRVSNLASGVPDRFSGSGSGTAFTLRISRVEAEDVGVYYCMQHLEYPYTFGGG
TRLEIK
77. anti-CD99 scFv
SEQ ID NO. 77
DVKLQESGAELARPGASVKLSCKASGYTFTTFWMQRVKQRPGQGLEWIGTIYPGDDDTRY
TQKFKGRATLTADKSSTTAYMQLSNLSSEDSAVYYCARSGYERGPYYFDSWGQGTTLTVS
SGGGGSGGGGSGGGGSDTVMSQSPSSLAVSVGEKITMSCKSSQSLLCRSNQKNYLAWYQQ
KPGQSPKQLIYWASTRESGVPDRFTGSGSGTDFTLTISSVKAEDLAVYYCQQYYSYPLTFG
AGTKLELK
78. anti-CD scFv
SEQ ID NO. 78
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDIQMTQSPASLSASVGETVTITCRASGNIHNYLAWYQQKQGKS
PQLLVYNAKTLADGVPSRFSGSGSGTQYSLKINSLQPEDFGSYYCQHFWSTPWTFGGGTKL
EIK
79. anti-CD99 scFv
SEQ ID NO. 79
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSGNSWSHSLRSLSVTIGQPASISCKSSQSLLDGNGKTYLNWLLQR
PGQSPKRLLYLVSKLDSGVPDRFTGSGSGTDFTLKISRVEAEDLGIYYCWQGTHFPRTFGGG
TKLEIK
80. anti-CD99 scFv
SEQ ID NO. 80
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDIVMTQAAPSVPVTPGESVSISCGSSKSLLHSNGNTYLYWFLQR
PGQSPQLLIYRVSNLASGVPDRFSGSGSGTAFTLRISRVEAEDVGVYYCMQHLEYPYTFGG
GTRLEIK
81. anti-CD99 scFv
SEQ ID NO. 81
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDTVMSQSPSSLAVSVGEKITMSCKSSQSLLCRSNQKNYLAWYQ
QKPGQSPKQLIYWASTRESGVPDRFTGSGSGTDFTLTISSVKAEDLAVYYCQQYYSYPLTF
GAGTKLELK
82. anti-CD99 scFv
SEQ ID NO. 82
QVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SSGGGGSGGGGSGGGGSDTVMSQSPSSLAVSVGEKITMSCKSSQSLLYRSNQKNYLAWYQ
QKPGQSPKQLIYWASTRESGVPDRFTGSGSGTDFTLTISSVKAEDLAVYYCQQYYSYPLTF
GAGTKLELK
83. anti-CD99, 1 H3H9 v2
SEQ ID NO. 83
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SEVQLQQSGAELVKPGASVKLSCTASGFDIKDTYIHVWKQRPEQGLEWIGRIDPANGDTRY
DPEFQGKASLTADTSSNTAYLQFSNLTSEDTAVYYCARRGGLSWGQGTTLTVSS
84. anti-CD99 scFv
SEQ ID NO. 84
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SEVQLEESGGGLVQPKGSLKLSCAASGFTFNTYAMYVWCQAPGKGLKVWARIRSKVNNY
ATYYADSVKDRFTISRDDSQNMLFLHMNNLKTEDTAIYFCVRDPMDYWGQGISVTVSS
85. antiCD99 scFv
SEQ ID NO. 85
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SEVQLVESGGGLVQPKGSLKLSCAASGFTFNTYAMYVWCQAPGKGLKVWARIRSKVNNY
ATYYADSVKDRFTISRDDSQNMLFLHMNNLKTEDTAIYFCVRDPMDYWGQGISVTVSS
86. antiCD99 scFv
SEQ ID NO. 86
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SQVQLQQSGAELARPGASVKLSCKASGYTFTTFWMQWVKQRPGQGLEWIGTIYPGDDDT
RYTQKFKGRATLTADKSSTTAYMQLSNLSSEDSAVYYCARSGYERGPYYFDSWGQGTTLT
VSS
87. anti-CD99 scFv
SEQ ID NO. 87
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SDVKLQESGAELARPGASVKLSCKASGYTFTTFWMQRVKQRPGQGLEWIGTIYPGDDDTR
YTQKFKGRATLTADKSSTTAYMQLSNLSSEDSAVYYCARSGYERGPYYFDSWGQGTTLTV
SS
88. anti-CD99 scFv
SEQ ID NO. 88
DIQMTQSPASLSASVGETVTITCRASGNIHNYLAWYQQKQGKSPQLLVYNAKTLADGVPS
RFSGSGSGTQYSLKINSLQPEDFGSYYCQHFWSTPWTFGGGTKLEIKGGGGSGGGGSGGGG
SQVQLKESGAELARPGASVKLSCKASGYTFTTFWMQWAKQRPGQGLEWIGTIYPGDDDTR
YTQKFKGRATLTADKSSTTAYMQLSNLSSEDSAVYYCARSGYERGPYYFDSWGQGTTLTV
SS
89. anti-CD99 scFv
SEQ ID NO. 89
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSEVQLQQSGAELVKPGASVKLSCTASGFDIKDTYIHWVKQRPEQGLEWIGRIDPANGD
TRYDPEFQGKASLTADTSSNTAYLQFSNLTSEDTAVYYCARRGGLSWGQGTTLTVSS
90. anti-CD99, 4C5E2 M2
SEQ ID NO. 90
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSEVQLEESGGGLVQPKGSLKLSCAASGFTFNTYAMYWVCQAPGKGLKWVARIRSKV
NNYATYYADSVKDRFTISRDDSQNMLFLHMNNLKTEDTAIYFCVRDPMDYWGQGISVTVS
S
91. anti-CD99, 4C5H10 M2
SEQ ID NO. 91
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSEVQLVESGGGLVQPKGSLKLSCAASGFTFNTYAMYWVCQAPGKGLKWVARIRSKV
NNYATYYADSVKDRFTISRDDSQNMLFLHMNNLKTEDTAIYFCVRDPMDYWGQGISVTVS
S
92. anti-CD99 scFv
SEQ ID NO. 92
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSQVQLQQSGAELARPGASVKLSCKASGYTFTTFWMQVWKQRPGQGLEWIGTIYPGD
DDTRYTQKFKGRATLTADKSSTTAYMQLSNLSSEDSAVYYCARSGYERGPYYFDSWGQG
TTLTVSS
93. anti-CD99 scFv
SEQ ID NO. 93
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSDVKLQESGAELARPGASVKLSCKASGYTFTTFWMQRVKQRPGQGLEWIGTIYPGDD
DTRYTQKFKGRATLTADKSSTTAYMQLSNLSSEDSAVYYCARSGYERGPYYFDSWGQGTT
LTVSS
94. anti-CD99 scFv
SEQ ID NO. 94
DIVMTQAAPSVPVTPGESVSISCGSSKSLLHSNGNTYLYWFLQRPGQSPQLLIYRVSNLASG
VPDRFSGSGSGTAFTLRISRVEAEDVGVYYCMQHLEYPYTFGGGTRLEIKGGGGSGGGGSG
GGGSQVQLKESGAELARPGASVKLSCKASGYTFTTFWMQWAKQRPGQGLEWIGTIYPGD
DDTRYTQKFKGRATLTADKSSTTAYMQLSNLSSEDSAVYYCARSGYERGPYYFDSWGQG
TTLTVSS
95. anti-CD99 scFv
SEQ ID NO. 95
DTVMSQSPSSLAVSVGEKITMSCKSSQSLLCRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSEVQLQQSGAELVKPGASVKLSCTASGFDIKDTYIHWVKQRPEQGLEWIGRIDPA
NGDTRYDPEFQGKASLTADTSSNTAYLQFSNLTSEDTAVYYCARRGGLSWGQGTTLTVSS
96. anti-CD99 scFv
SEQ ID NO. 96
DTVMSQSPSSLAVSVGEKITMSCKSSQSLLCRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSEVQLEESGGGLVQPKGSLKLSCAASGFTFNTYAMYWVCQAPGKGLKWVARIR
SKVNNYATYYADSVKDRFTISRDDSQNMLFLHMNNLKTEDTAIYFCVRDPMDYWGQGIS
VTVSS
97. anti-CD99, 9G12C9 M2
SEQ ID NO. 97
DTVMSQSPSSLAVSVGEKITMSCKSSQSLLCRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSQVQLQQSGAELARPGASVKLSCKASGYTFTTFWMQWVKQRPGQGLEWIGTIY
PGDDDTRYTQKFKGRATLTADKSSTTAYMQLSNLSSEDSAVYYCARSGYERGPYYFDSW
GQGTTLTVSS
98. anti-CD99 scFv
SEQ ID NO. 98
DTVMSQSPSSLAVSVGEKITMSCKSSQSLLCRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSDVKLQESGAELARPGASVKLSCKASGYTFTTFWMQRVKQRPGQGLEWIGTIYP
GDDDTRYTQKFKGRATLTADKSSTTAYMQLSNLSSEDSAVYYCARSGYERGPYYFDSWG
QGTTLTVSS
99. anti-CD99 scFv
SEQ ID NO. 99
DTVMSQSPSSLAVSVGEKITMSCKSSQSLLCRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSQVQLKESGAELARPGASVKLSCKASGYTFTTFWMQWAKQRPGQGLEWIGTIYP
GDDDTRYTQKFKGRATLTADKSSTTAYMQLSNLSSEDSAVYYCARSGYERGPYYFDSWG
QGTTLTVSS
100. anti-CD99 scFv
SEQ ID NO. 100
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSEVQLQQSGAELVKPGASVKLSCTASGFDIKDTYIHWVKQRPEQGLEWIGRIDPA
NGDTRYDPEFQGKASLTADTSSNTAYLQFSNLTSEDTAVYYCARRGGLSWGQGTTLTVSS
101. anti-CD99 scFv
SEQ ID NO. 101
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSEVQLEESGGGLVQPKGSLKLSCAASGFTFNTYAMYWVCQAPGKGLKWVARIR
SKVNNYATYYADSVKDRFTISRDDSQNMLFLHMNNLKTEDTAIYFCVRDPMDYWGQGIS
VTVSS
102. anti-CD99 scFv
SEQ ID NO. 102
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSEVQLVESGGGLVQPKGSLKLSCAASGFTFNTYAMYWVCQAPGKGLKVWARIR
SKVNNYATYYADSVKDRFTISRDDSQNMLFLHMNNLKTEDTAIYFCVRDPMDYWGQGIS
VTVSS
103. anti-CD99 scFv
SEQ ID NO. 103
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSQVQLQQSGAELARPGASVKLSCKASGYTFTTFWMQWVKQRPGQGLEWIGTIY
PGDDDTRYTQKFKGRATLTADKSSTTAYMQLSNLSSEDSAVYYCARSGYERGPYYFDSW
GQGTTLTVSS
104. anti-CD99 scFv
SEQ ID NO. 104
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSDVKLQESGAELARPGASVKLSCKASGYTFTTFWMQRVKQRPGQGLEWIGTIYP
GDDDTRYTQKFKGRATLTADKSSTTAYMQLSNLSSEDSAVYYCARSGYERGPYYFDSWG
QGTTLTVSS
105. anti-CD99 scFv
SEQ ID NO. 105
DTVMSQSPSSLAVSVGEKITMSCKSSQSLLYRSNQKNYLAWYQQKPGQSPKQLIYWASTR
ESGVPDRFTGSGSGTDFTLTISSVKAEDLAVYYCQQYYSYPLTFGAGTKLELKGGGGSGGG
GSGGGGSQVQLKESGAELARPGASVKLSCKASGYTFTTFWMQWAKQRPGQGLEWIGTIYP
GDDDTRYTQKFKGRATLTADKSSTTAYMQLSNLSSEDSAVYYCARSGYERGPYYFDSWG
QGTTLTVSS
106. anti-CLEC12A, 1 F3H8
SEQ ID NO. 106
ELILVESGGGLVKPGGSLKLSCAVSGFTFSSFAMSVWRQTPEKRLEVWATISSGGAYTFYK
DSVKGRFTISRDNAKNTLYLQMSSLRSEDSAMYYCARHSGYDGYYLYAMDYWGQGTSVT
VSS
107. anti-CLEC12A, 1 F3H8
SEQ ID NO. 107
GAACTAATACTGGTGGAGTCTGGGGGAGGCTTAGTGAAGCCTGGAGGGTCCCTGA
AACT CT COT GTGCAGT CT CTGGATT CACTTT CAGTT CCTTTGCCAT GT CTT GGGTT
C GCCAGACT CCGGAGAAGAGGCT GGAGT GGGT CGCAACCATT AGTAGT GGTGGAGC
TT ACACCTT CT AT AAAG ACAGT GT G AAGGGGCGATT CACCAT CT CCAG AGACAAT
G CCAAG AAT ACCCTGTACCTG CAAAT GAG CAGT CT G AG GT CT G AGG ACT CG G
COAT GT ATT ACT GT GCAAG ACAT AGCGGCT AT GAT GGTT ACT ACCT CT ATGCT
ATGGACT AC TGGGGT CAAGG AACCT CAGT CACCGT CT CCT CA
108. anti-CLEC12A, 1 F3A10
SEQ ID NO. 108
GVQCELILVESGGGLVKPGGSLKLSCAVSGFTFSSFAVSVWRQTPEKRLEVWATISSGGAY
TFYKDSVKGRFTISRDNAKNTLYLQMSSLRSEDSAMYYCARHSGYDGYYLYAMD
YWGQGTSVTVSS
109. anti-CLEC12A, 1 F3A10
SEQ ID NO. 109
GGTGTCCAGTGTGAACTAATACTGGTGGAGTCTGGGGGAGGCTTAGTGAAGCCTGGAG
GGTCCCTGAAACTCTCCTGTGCAGTCTCTGGATTCACTTTCAGTTCCTTTGCCGTGTCCT
GGGTTCGCCAGACTCCGGAGAAGAGGCTGGAGTGGGTCGCAACCATTAGTAGTGGTGG
AGCTTACACCTTCTATAAAGACAGTGTGAAGGGGCGATTCACCATCTCCAGAGACAAT
GCCAAGAATACCCTGTACCTGCAAATGAGCAGTCTGAGGTCTGAGGACTCGGCCATGT
ATTACTGTGCAAGACATAGCGGCTATGATGGTTACTACCTCTATGCTATGGACTACTGG
GGTCAAGGAACCTCAGTCACCGTCTCCTCA
110. anti-CLEC12A, 1 F3F3
SEQ ID NO. 110
EVQLEESGGGLVQPGGSLKVSCAVSGLAFSSHDMSVWRQTPEKRLEVWAYISGGGTNIYY
SDTVKGRFTISRDNAKNTLYLQMSSLKSEDTAIYYCARPNYNYGGSWFAYWGQG
TLVTVSA
111. anti-CLEC12A, 1 F3F3
SEQ ID NO. 111
GAGGTGCAGCTGGAGGAGTCTGGGGGAGGCTTAGTGCAGCCGGGAGGGTCCCTGAAA
GTCTCCTGTGCAGTTTCCGGACTCGCTTTCAGCAGCCATGACATGTCTTGGGTTCGCCA
GACTCCGGAGAAGCGGCTGGAGTGGGTCGCATACATTAGTGGAGGTGGTACTAATATC
TATTATTCAGACACTGTGAAGGGCCGATTCACCATCTCCAGAGACAATGCCAAGAACA
CCCTGTACCTGCAAATGAGCAGTCTGAAGTCTGAAGACACAGCCATTTATTACTGTGCA
AGACCCAATTATAATTACGGCGGTTCCTGGTTTGCTTACTGGGGCCAAGGGACTCTGGT
CACTGTCTCTGCA
112. anti-CLEC12A, 1 F3H8, 1 F3F3, 1 F3A1 G
SEQ ID NO. 112
QIVLTQSPEIMSASPGEKVTMTCSASSSVHYMHWYQQKSGTSPKRWIYDTSKLASGVP
GRFSGSGSGTSYSLTISSMESEDAATYYCQQWTSNPPTFGGGTKLEIK
113. anti-CLEC12A, 1 F3H8, 1 F3F3, 1 F3A1 Q
SEQ ID NO. 113
CAAATTGTTCTCACCCAGTCTCCAGAAATCATGTCTGCATCTCCAGGGGAGAAGGTCAC
CATGACCTGCAGTGCCAGCTCAAGTGTACATTACATGCACTGGTACCAGCAGAAGTCA
GGCACCTCCCCCAAAAGATGGATTTATGACACATCCAAACTGGCTTCTGGAGTCCCTGG
TCGCTTCAGTGGCAGTGGGTCTGGGACCTCTTACTCTCTCACAATCAGCAGCATGGAGT
CTGAAGATGCTGCCACTTATTACTGCCAGCAGTGGACTAGTAACCCACCCACGTTOGGA
GGGGGGACCAAGCTGGAAATTAAACG
114. anti-CLEC12A, 1 F3H8 v1
SEQ ID NO. 114
ELILVESGGGLVKPGGSLKLSCAVSGFTFSSFAMSVWRQTPEKRLEVWATISSGGAYTFYK
DSVKGRFTISRDNAKNTLYLQMSSLRSEDSAMYYCARHSGYDGYYLYAMDYWGQGTSVT
VSSGGGGSGGGGSGGGGSQIVLTQSPEIMSASPGEKVTMTCSASSSVHYMHWYQQKSGTS
PKRWIYDTSKLASGVPGRFSGSGSGTSYSLTISSMESEDAATYYCQQWTSNPPTFGGGTKLE
IK
115. anti-CLEC12A, 1 F3A10 v1
SEQ ID NO. 115
GVQCELILVESGGGLVKPGGSLKLSCAVSGFTFSSFAVSVWRQTPEKRLEVWATISSGGAY
TFYKDSVKGRFTISRDNAKNTLYLQMSSLRSEDSAMYYCARHSGYDGYYLYAMDYWGQ
GTSVTVSSGGGGSGGGGSGGGGSQIVLTQSPEIMSASPGEKVTMTCSASSSVHYMHWYQQ
KSGTSPKRWIYDTSKLASGVPGRFSGSGSGTSYSLTISSMESEDAATYYCQQWTSNPPTFGG
GTKLEIK
116. anti-CLEC12A, 1 F3F3 v1
SEQ ID NO. 116
EVQLEESGGGLVQPGGSLKVSCAVSGLAFSSHDMSVWRQTPEKRLEVWAYISGGGTNIYY
SDTVKGRFTISRDNAKNTLYLQMSSLKSEDTAIYYCARPNYNYGGSWFAYWGQGTLVTVS
AGGGGSGGGGSGGGGSQIVLTQSPEIMSASPGEKVTMTCSASSSVHYMHWYQQKSGTSPK
RWIYDTSKLASGVPGRFSGSGSGTSYSLTISSMESEDAATYYCQQWTSNPPTFGGGTKLEIK
117. anti-CLEC12A, 1 F3H8 v2
SEQ ID NO. 117
QIVLTQSPEIMSASPGEKVTMTCSASSSVHYMHWYQQKSGTSPKRWIYDTSKLASGVPGRF
SGSGSGTSYSLTISSMESEDAATYYCQQWTSNPPTFGGGTKLEIKGGGGSGGGGSGGGGSE
LILVESGGGLVKPGGSLKLSCAVSGFTFSSFAMSVWRQTPEKRLEWVATISSGGAYTFYKD
SVKGRFTISRDNAKNTLYLQMSSLRSEDSAMYYCARHSGYDGYYLYAMDYWGQGTSVTV
SS
118. anti-CLEC12A, 1 F3A10 v2
SEQ ID NO. 118
QIVLTQSPEIMSASPGEKVTMTCSASSSVHYMHWYQQKSGTSPKRWIYDTSKLASGVPGRF
SGSGSGTSYSLTISSMESEDAATYYCQQWTSNPPTFGGGTKLEIKGGGGSGGGGSGGGGSG
VQCELILVESGGGLVKPGGSLKLSCAVSGFTFSSFAVSVWRQTPEKRLEWVATISSGGAYTF
YKDSVKGRFTISRDNAKNTLYLQMSSLRSEDSAMYYCARHSGYDGYYLYAMDYWGQGT
SVTVSS
119. anti-CLEC12A, 1 F3F3 v2
SEQ ID NO. 119
QIVLTQSPEIMSASPGEKVTMTCSASSSVHYMHWYQQKSGTSPKRWIYDTSKLASGVPGRF
SGSGSGTSYSLTISSMESEDAATYYCQQWTSNPPTFGGGTKLEIKGGGGSGGGGSGGGGSE
VQLEESGGGLVQPGGSLKVSCAVSGLAFSSHDMSWVRQTPEKRLEVWAYISGGGTNIYYS
DTVKGRFTISRDNAKNTLYLQMSSLKSEDTAIYYCARPNYNYGGSWFAYWGQGTLVTVS
A
120. anti-CD33 scFv VH domain
SEQ ID NO. 120
EVKLVESGGGLVKPGASLKLSCAASGFTFSNYGMSVNRQTSDKRLEVWASISSGGGDTYY
PDNVKGRFTISRENAKNTLYLQMSSLNSEDTALYYCARDYGGTWDYFDYW
GQGTTLTVSS
121. anti-CD33 scFv VH domain
SEQ ID NO. 121
QVQLQQPGAELVRPGVSVKLSCKASGYTFTSYWMNWVKQRPGQGLEWIGMIHPSDSETR
LNQKFKDKAILTVDKSSSTAYMQLSSPTSEDSAVYYCAREEGQLGHGGAM
DYWGQGTSVTVSS
122. anti-CD33 scFv VH domain
SEQ ID NO. 122
QVQLKESGPGLVAPSQSLSITCTVSGFSLSRYSVHW\VRQPPGKGLEWLGMIWGGGYTDYN
SALKSRLSISKDNSKSQVFLKMNSLQTDDTAMYYCARYIDSSGYDYWGQG TTLTVSS
123. anti-CD33 scFv VL domain
SEQ ID NO. 123
DIQMTQTTSSLSASLGDRVTISCRASQDISKYLNWYQQKPDGTVKLLIYYTSRLHSG
VPSRFSGSGSGTDYSLTISNLEQEDIATYFCQQGDTFPWTFGGGTKLEIK
124. anti-CD33 scFv VL domain
SEQ ID NO. 124
SIVMTQTPKFLLVSAGDRVTITCKASQTVNDDVAWYQQKPGQSPKLLIYYVSNRHTGVPD
RFTGSGYGTDFTFTISTVQAEDLAVYFCQQDYSSPYTFGGGTKLEIK
125. anti-CD33 scFv VL domain
SEQ ID NO. 125
QIVLTQSPAIMSASPGEKVTMTCSASSSVSYMHWYQQKSGTSPKRWIYDTSKLASG
VPARFSGSGSGTSYSLTISSMEAEDAATYYCQQWSSNPLTFGAGTKLELK
126. anti-CD33 scFv VL domain
SEQ ID NO. 126
DIQMTQSPASLSASVGETVTITCRASENIYSYLAWYQQKQGKSPQLLVYNAKTLAEG
VPSRFSGSGSGTQFSLKINSLQPEDFGSYYCQHHYGTPYTFGGGTKLEIK
127. anti-CD33 6A11HC1_LC
SEQ ID NO. 127
EVKLVESGGGLVKPGASLKLSCAASGFTFSNYGMSVNRQTSDKRLEVWASISSGGGDTYY
PDNVKGRFTISRENAKNTLYLQMSSLNSEDTALYYCARDYGGTWDYFDYWGQGTTLTVS
SGGGGSGGGGSGGGGSDIQMTQTTSSLSASLGDRVTISCRASQDISKYLNWYQQKPDGTV
KLLIYYTSRLHSGVPSRFSGSGSGTDYSLTISNLEQEDIATYFCQQGDTFPWTFGGGTKLEIK
128. anti-CD33 6A11HC2_LC
SEQ ID NO. 128
QVQLQQPGAELVRPGVSVKLSCKASGYTFTSYWMNWVKQRPGQGLEWIGMIHPSDSETR
LNQKFKDKAILTVDKSSSTAYMQLSSPTSEDSAVYYCAREEGQLGHGGAMDYWGQGTSV
TVSSGGGGSGGGGSGGGGSDIQMTQTTSSLSASLGDRVTISCRASQDISKYLNWYQQKPDG
TVKLLIYYTSRLHSGVPSRFSGSGSGTDYSLTISNLEQEDI
ATYFCQQGDTFPWTFGGGTKLEIK
129. anti-CD33 27A3HCLC1
SEQ ID NO. 129
QVQLKESGPGLVAPSQSLSITCTVSGFSLSRYSVHWVRQPPGKGLEWLGMIWGGGYTDYN
SALKSRLSISKDNSKSQVFLKMNSLQTDDTAMYYCARYIDSSGYDYWGQGTTLTVSSGGG
GSGGGGSGGGGSSIVMTQTPKFLLVSAGDRVTITCKASQTVNDDVAWYQQKPGQSPKLLI
YYVSNRHTGVPDRFTGSGYGTDFTFTISTVQAEDLAVYFCQ QDYSSPYTFGGGTKLEIK
130. anti-CD33 27A3HCLC2
SEQ ID NO. 130
QVQLKESGPGLVAPSQSLSITCTVSGFSLSRYSVHV\RQPPGKGLEWLGMIWGGG YTDYNS
ALKSRLSISKDNSKSQVFLKMNSLQTDDTAMYYCARYIDSSGYDYWGQGTTLTVSSGGGG
SGGGGSGGGGSQIVLTQSPAIMSASPGEKVTMTCSASSSVSYMHWYQQKSGTSPKRWIYD
TSKLASGVPARFSGSGSGTSYSLTISSMEAEDAATYYCQ QWSSNPLTFGAGTKLELK
131. anti-CD33 27A3HCLC3
SEQ ID NO. 131
QVQLKESGPGLVAPSQSLSITCTVSGFSLSRYSVHVWRQPPGKGLEWLGMIWGGGYTDYN
SALKSRLSISKDNSKSQVFLKMNSLQTDDTAMYYCARYIDSSGYDYWGQGTTLTVSSGGG
GSGGGGSGGGGSDIQMTQSPASLSASVGETVTITCRASENIYSYLAWYQQKQGKSPQLLVY
NAKTLAEGVPSRFSGSGSGTQFSLKINSLQPEDFGSYYCQ HHYGTPYTFGGGTKLEIK
132. anti-CD33 6A11HC1_LC
SEQ ID NO. 132
ATGGCCCTCCCGGTAACGGCTCTGCTGCTTCCACTCGCACTGCTCTTGCATGCTGCCAG
ACCAGAAGTGAAGCTGGTGGAGTCTGGGGGAGGCTTAGTGAAGCCTGGAGCGTCTCTG
AAACTCTCCTGTGCAGCCTCTGGATTCACTTTCAGTAACTATG
GCATGTCTTGGGTTCGCCAGACTTCAGACAAGAGGCTGGAGTGGGTCGCATCC
ATTAGTAGTGGTGGTGGTGACACCTACTATCCAGACAATGTAAAGGGCCGATTC
ACCATCTCCAGAGAGAATGCCAAGAACACCCTGTACCTGCAAATGAGTAGTCTG
AACTCTGAGGACACGGCCTTGTATTACTGTGCAAGAGACTATGGTGGTACTTGG
GACTACTTTGACTACTGGGGCCAAGGCACCACTCTCACAGTCTCCTCAGGTGGA
GGTGGATCAGGTGGAGGTGGATCTGGTGGAGGTGGATCTGATATCCAGATGAC
ACAGACTACATCCTCCCTGTCTGCCTCTCTGGGAGACAGAGTCACCATCAGTTG
CAGGGCAAGTCAGGACATTAGCAAGTATTTAAACTGGTATCAGCAGAAACCAGATGGA
ACTGTTAAACTCCTGATCTACTACACATCAAGATTACACTCAGGAGTCCCATCGAGGTT
CAGTGGCAGTGGGTCTGGAACAGATTATTCTCTCACCATTAGCAACCTGGAGCAAGAA
GATATTGCCACTTACTTTTGCCAACAGGGTGATACGTTTCCG
TGGACGTTCGGTGGAGGCACCAAGCTGGAAATCAAACGG
133. anti-CD33 6A11HC2_LC
SEQ ID NO. 133
ATGGCCCTCCCGGTAACGGCTCTGCTGCTTCCACTCGCACTGCTCTTGCATGCTGCCAG
ACCACAGGTCCAACTGCAGCAGCCTGGGGCTGAGCTGGTGAGGCCTGGAGTTTCAGTG
AAGCTGTCCTGCAAGGCTTCTGGCTACACCTTCACCAG
CTACTGGATGAACTGGGTGAAGCAGAGGCCTGGACAAGGCCTTGAGTGGATTG
GCATGATTCATCCTTCCGATAGTGAAACTAGGTTAAATCAGAAGTTCAAGGACAA GGC
CATATTGACTGTAGACAAATCCTCCAGCACAGCCTACATGCAACTCAGCAGCCCGACA
TCTGAGGACTCTGCGGTCTATTACTGTGCAAGAGAAGAGGGACAGC
TCGGGCACGGCGGTGCTATGGACTACTGGGGTCAAGGAACCTCAGTCACCGTC
TCCTCAGGTGGAGGTGGATCAGGTGGAGGTGGATCTGGTGGAGGTGGATCTGA
TATCCAGATGACACAGACTACATCCTCCCTGTCTGCCTCTCTGGGAGACAGAGT
CACCATCAGTTGCAGGGCAAGTCAGGACATTAGCAAGTATTTAAACTGGTATCA
GCAGAAACCAGATGGAACTGTTAAACTCCTGATCTACTACACATCAAGATTACACTCA
GGAGTCCCATCGAGGTTCAGTGGCAGTGGGTCTGGAACAGATTATTCTCTCACCATTAG
CAACCTGGAGCAAGAAGATATTGCCACTTACTTTTGCCAACAGGGT
GATACGTTTCCGTGGACGTTCGGTGGAGGCACCAAGCTGGAAATCAAACGG
134. anti-CD33 27A3HC_LC1
SEQ ID NO. 134
ATGGCCCTCCCGGTAACGGCTCTGCTGCTTCCACTCGCACTGCTCTTGC
ATGCTGCCAGACCACAGGTGCAGCTGAAGGAGTCAGGACCTGGCCTGGTGGC
ACCCTCACAGAGCCTGTCCATCACATGCACGGTCTCTGGGTTCTCATTATCCAG
ATATAGTGTACACTGGGTTCGCCAGCCTCCAGGAAAGGGTCTGGAGTGGCTGG
GAATGATATGGGGTGGTGGATACACAGACTATAATTCAGCTCTCAAATCCAGAC
TGAGCATCAGCAAGGACAACTCCAAGAGCCAAGTTTTCTTAAAAATGAACAGTC
TGCAAACTGATGACACAGCCATGTACTACTGTGCCAGATATATAGACAGCTCGG
GCTACGACTACTGGGGCCAAGGCACCACTCTCACAGTCTCCTCAGGTGGAGGT
GGATCAGGTGGAGGTGGATCTGGTGGAGGTGGATCTAGTATTGTGATGACCCA
GACTCCCAAATTCCTGCTTGTATCAGCAGGAGACAGGGTTACCATAACCTGCAA
GGCCAGTCAGACTGTGAATGATGATGTAGCTTGGTATCAACAGAAGCCAGGACA
GTCTCCTAAATTGCTGATATATTATGTATCCAATCGCCACACTGGAGTCCCTGAT
CGCTTCACTGGCAGTGGATATGGGACGGATTTCACTTTCACCATCAGCACTGTG
CAGGCTGAAGACCTGGCAGTTTATTTCTGTCAGCAGGATTATAGCTCTCCGTAC
ACGTTCGGAGGGGGGACCAAGCTGGAAATAAAACGG
135. anti-CD33 27A3HCLC2
SEQ ID NO. 135
ATGGCCCTCCCGGTAACGGCTCTGCTGCTTCCACTCGCACTGCTCTTGC
ATGCTGCCAGACCACAGGTGCAGCTGAAGGAGTCAGGACCTGGCCTGGTGGC
ACCCTCACAGAGCCTGTCCATCACATGCACGGTCTCTGGGTTCTCATTATCCAG
ATATAGTGTACACTGGGTTCGCCAGCCTCCAGGAAAGGGTCTGGAGTGGCTGG
GAATGATATGGGGTGGTGGATACACAGACTATAATTCAGCTCTCAAATCCAGAC TGAG
CATCAGCAAGGACAACTCCAAGAGCCAAGTTTTCTTAAAAATGAACAGTC
TGCAAACTGATGACACAGCCATGTACTACTGTGCCAGATATATAGACAGCTCGG
GCTACGACTACTGGGGCCAAGGCACCACTCTCACAGTCTCCTCAGGTGGAGGT
GGATCAGGTGGAGGTGGATCTGGTGGAGGTGGATCTCAAATTGTTCTCACCCA
GTCTCCAGCAATCATGTCTGCATCTCCAGGGGAGAAGGTCACCATGACCTGCA
GTGCCAGCTCAAGTGTAAGTTACATGCACTGGTACCAGCAGAAGTCAGGCACCT
CCCCCAAAAGATGGATTTATGACACATCCAAACTGGCTTCTGGAGTCCCTGCTC
GCTTCAGTGGCAGTGGGTCTGGGACCTCTTACTCTCTCACAATCAGCAGCATGG
AGGCTGAAGATGCTGCCACTTATTACTGCCAGCAGTGGAGTAGTAACCCACTCA
CGTTCGGTGCTGGGACCAAGCTGGAGCTGAAACGG
136. anti-CD33 27A3HCLC3
SEQ ID NO. 136
ATGGCCCTCCCGGTAACGGCTCTGCTGCTTCCACTCGCACTGCTCTTGCATGCT
GCCAGACCACAGGTGCAGCTGAAGGAGTCAGGACCTGGCCTGGTGGCACCCT
CACAGAGCCTGTCCATCACATGCACGGTCTCTGGGTTCTCATTATCCAGATATA
GTGTACACTGGGTTCGCCAGCCTCCAGGAAAGGGTCTGGAGTGGCTGGGAATG
ATATGGGGTGGTGGATACACAGACTATAATTCAGCTCTCAAATCCAGACTGAGC
ATCAGCAAGGACAACTCCAAGAGCCAAGTTTTCTTAAAAATGAACAGTCTGCAAACTG
ATGACACAGCCATGTACTACTGTGCCAGATATATAGACAGCTCGGGCTACG
ACTACTGGGGCCAAGGCACCACTCTCACAGTCTCCTCAGGTGGAGGTGGATCA
GGTGGAGGTGGATCTGGTGGAGGTGGATCTGACATCCAGATGACTCAGTCTCC
AGCCTCCCTATCTGCATCTGTGGGAGAAACTGTCACCATCACATGTCGAGCAAG
TGAGAATATTTACAGTTATTTAGCATGGTATCAGCAGAAACAGGGAAAATCTCCT
CAGCTCCTGGTCTATAATGCAAAAACCTTAGCAGAAGGTGTGCCATCAAGGTTC
AGTGGCAGTGGATCAGGCACACAGTTTTCTCTGAAGATCAACAGTCTGCAGCCT
GAAGATTTTGGGAGTTATTACTGTCAACATCATTATGGTACTCCGTACACGTTCG
GAGGGGGGACCAAGCTGGAAATAAAACGG
137. anti-CD33 CDR1 V_{H} domain
SEQ ID NO: 137
GFTFSNYG
138. anti-CD33 CDR1 V_{H} domain
SEQ ID NO: 138
GYTFTSYW
139. anti-CD33 CDR1 V_{H} domain
SEQ ID NO: 139
GFSLSRYS
140. anti-CD33 CDR2 V_{H} domain
SEQ ID NO: 140
ISSGGGDT
141. anti-CD33 CDR2 V_{H} domain
SEQ ID NO: 141
HPSDSET
142. anti-CD33 CDR2 V_{H} domain
SEQ ID NO: 142
IWGGGYT
143. anti-CD33 CDR3 V_{H} domain
SEQ ID NO: 143
ARDYGGTWDYFDY
144. anti-CD33 CDR3 V_{H} domain
SEQ ID NO: 144
AREEGQLGHGGAMDY
145. anti-CD33 CDR3 V_{H} domain
SEQ ID NO: 145
ARYIDSSGYDY
146. anti-CD33 CDR1 V_{L} domain
SEQ ID NO: 146
QDISKY
147. anti-CD33 CDR1 V_{L} domain
SEQ ID NO: 147
QTVNDD
148. anti-CD33 CDR1 V_{L} domain
SEQ ID NO: 148
SSVSY
149. anti-CD33 CDR1 V_{L} domain
SEQ ID NO: 149
ENIYSY
150. anti-CD33 CDR2 V_{L} domain; x refers to amino acid
SEQ ID NO: 150
YTSx
151. anti-CD33 CDR2 V_{L} domain; x refers to amino acid
SEQ ID NO: 151
YVSx
152. anti-CD33 CDR2 V_{L} domain; x refers to amino acid
SEQ ID NO: 152
DTSx
153. anti-CD33 CDR2 V_{L} domain; x refers to amino acid
SEQ ID NO: 153
NAKx
154. anti-CD33 CDR3 V_{L} domain
SEQ ID NO: 154
QQGDTFPWT
155. anti-CD33 CDR3 V_{L} domain
SEQ ID NO: 155
QQDYSSPYT
156. anti-CD33 CDR3 V_{L} domain
SEQ ID NO: 156
QQWSSNPLT
157. anti-CD33 CDR3 V_{L} domain
SEQ ID NO: 157
QHHYGTPYT
158. anti-CD123 scFv CDR1 V_{H} domain
SEQ ID NO: 158
GYTFTDYN
159. anti-CD123 scFv CDR2 V_{H} domain
SEQ ID NO: 159
INPNNGGT
160. anti-CD123 scFv CDR3 V_{H} domain
SEQ ID NO: 160
ARKGYGGNYDYFDY
161. anti-CD123 scFv CDR1 V_{L} domain
SEQ ID NO: 161
QSIGTS
162. anti-CD123 scFv CDR2 V_{L} domain; x refers to amino acid
SEQ ID NO: 162
YASx
163. anti-CD123 scFv CDR3 V_{L} domain
SEQ ID NO: 163
QQSNSWPYT
164. anti-CD123 scFv CDR1 V_{H} domain
SEQ ID NO: 164
GFNIKDTY
165. anti-CD123 scFv CDR1 V_{H} domain
SEQ ID NO: 165
GFSLSTYGMG
166. anti-CD123 scFv CDR2 V_{H} domain
SEQ ID NO: 166
IDPANGNT
167. anti-CD123 scFv CDR2 V_{H} domain
SEQ ID NO: 167
LYWDDDK
168. anti-CD123 scFv CDR3 V_{H} domain
SEQ ID NO: 168
ALYYYGGSLDY
169. anti-CD123 scFv CDR3 V_{H} domain
SEQ ID NO: 169
AQSLIYDGYYGFAY
170. anti-CD123 scFv CDR1 V_{L} domain
SEQ ID NO: 170
QSLLYSGNQKNY
171. anti-CD123 scFv CDR2 V_{L} domain; x refers to amino acid
SEQ ID NO: 171
WASx
172. anti-CD123 scFv CDR3 V_{L} domain
SEQ ID NO: 172
QQYYSYPRT
173. anti-CD123 scFv CDR1 V_{H} domain
SEQ ID NO: 173
GYTFTYYG
174. anti-CD123 scFv CDR2 V_{H} domain
SEQ ID NO: 174
INTYSGVP
175. anti-CD123 scFv CDR3 V_{H} domain
SEQ ID NO: 175
ARWIYYSDLYGMDY
176. anti-CD123 scFv CDR1 V_{L} domain
SEQ ID NO: 176
QSIVHSNGDTY
177. anti-CD123 scFv CDR2 V_{L} domain; x refers to amino acid
SEQ ID NO: 177
KVSx
178. anti-CD123 scFv CDR3 V_{L} domain
SEQ ID NO: 178
FQGSHVPWT
179. anti-CD123 scFv CDR1 V_{H} domain
SEQ ID NO: 179
GYTFSSYW
180. anti-CD123 scFv CDR1 V_{H} domain
SEQ ID NO: 180
GYTLTTYL
181. anti-CD123 scFv CDR2 V_{H} domain
SEQ ID NO: 181
INPSSGYT
182. anti-CD123 scFv CDR2 V_{H} domain
SEQ ID NO: 182
INPNSGSS
183. anti-CD123 scFv CDR3 V_{H} domain
SEQ ID NO: 183
ARDGNYDHWYFDV
184. anti-CD123 scFv CDR3 V_{H} domain
SEQ ID NO: 184
AIRHYGGSLFDY
185. anti-CD123 scFv CDR1 V_{L} domain
SEQ ID NO: 185
QDINSY
186. anti-CD123 scFv CDR1 V_{L} domain
SEQ ID NO: 186
QSLLNSRTRKNY
187. anti-CD123 scFv CDR2 V_{L} domain; x refers to amino acid
SEQ ID NO: 187
RANx
188. anti-CD123 scFv CDR3 V_{L} domain
SEQ ID NO: 188
LQYDELLT
189. anti-CD123 scFv CDR3 V_{L} domain
SEQ ID NO: 189
EQSYNLFT
190. anti-CD99 scFv CDR1 V_{H} domain
SEQ ID NO: 190
GFDIKDTY
191. anti-CD99 scFv CDR1 V_{H} domain
SEQ ID NO: 191
TYAMY
192. anti-CD99 scFv CDR1 V_{H} domain
SEQ ID NO: 192
TFWM
193. anti-CD99 scFv CDR1 V_{H} domain
SEQ ID NO: 193
TFWMQ
194. anti-CD99 scFv CDR2 V_{H} domain
SEQ ID NO: 194
IDPANGDT
195. anti-CD99 scFv CDR2 V_{H} domain
SEQ ID NO: 195
RIRSKVNNYATYYADSVKDRFT
196. anti-CD99 scFv CDR2 V_{H} domain
SEQ ID NO: 196
TIYPGDDDTRYTQKFKGRAT
197. anti-CD99 scFv CDR3 V_{H} domain
SEQ ID NO: 197
ARRGGLS
198. anti-CD99 scFv CDR3 V_{H} domain
SEQ ID NO: 198
DPMDY
199. anti-CD99 scFv CDR3 V_{H} domain
SEQ ID NO: 199
SGYERGPYYFDS
200. anti-CD99 scFv CDR3 V_{H} domain
SEQ ID NO: 200
SGYERGPYYF
201. anti-CD99 scFv CDR1 V_{L} domain
SEQ ID NO: 201
GNIHNY
202. anti-CD99 scFv CDR1 V_{L} domain
SEQ ID NO: 202
GSSKSLLHSNGNTYLY
203. anti-CD99 scFv CDR1 V_{L} domain
SEQ ID NO: 203
KSSQSLLCRSNQKNYLA
204. anti-CD99 scFv CDR1 V_{L} domain
SEQ ID NO: 204
KSSQSLLYRSNQKNYLA
205. anti-CD99 scFv CDR2 V_{L} domain
SEQ ID NO: 205
RVSNLAS
206. anti-CD99 scFv CDR2 V_{L} domain
SEQ ID NO: 206
WASTRES
207. anti-CD99 scFv CDR3 V_{L} domain
SEQ ID NO: 207
QHFWSTPWT
208. anti-CD99 scFv CDR3 V_{L} domain
SEQ ID NO: 208
MQHLEYPYT
209. anti-CD99 scFv CDR3 V_{L} domain
SEQ ID NO: 209
QQYYSYPLT
210. anti-CLEC12A CDR1 V_{H} domain
SEQ ID NO: 210
GFTFSSFA
211. anti-CLEC12A CDR1 V_{H} domain
SEQ ID NO: 211
SFAVS
212. anti-CLEC12A CDR1 V_{H} domain
SEQ ID NO: 212
SHDMS
213. anti-CLEC12A CDR2 V_{H} domain
SEQ ID NO: 213
ISSGGAYT
214. anti-CLEC12A CDR2 V_{H} domain
SEQ ID NO: 214
TISSGGAYTFYKDSVKGRFT
215. anti-CLEC12A CDR2 V_{H} domain
SEQ ID NO: 215
YISGGGTNIYYSDTVKGRFT
216. anti-CLEC12A CDR3 V_{H} domain
SEQ ID NO: 216
ARHSGYDGYYLYAMDY
217. anti-CLEC12A CDR3 V_{H} domain
SEQ ID NO: 217
HSGYDGYYLYAMDY
218. anti-CLEC12A CDR3 V_{H} domain
SEQ ID NO: 218
PNYNYGGSWFAY
219. anti-CLEC12A CDR1 V_{L} domain
SEQ ID NO: 219
SSVHY
220. anti-CLEC12A CDR1 V_{L} domain
SEQ ID NO: 220
ASSSVHYMH
221. anti-CLEC12A CDR1 V_{L} domain
SEQ ID NO: 221
SASSSVHYMH
222. anti-CLEC12A CDR2 V_{L} domain
SEQ ID NO: 222
DTSKLAS
223. anti-CLEC12A CDR3 V_{L} domain
SEQ ID NO: 223
QQWTSNPPT

1. A bispecific chimeric antigen receptor (CAR) polypeptide comprising at least two tumor associated antigen (TAA) binding domains, wherein the TAA binding domains are selected from the group comprising an anti-CD33 binding domain, an anti-CLEC12A binding domain, an anti-CD123 binding domain, an anti-CD99 binding domain, or any combinations thereof.

2. The bispecific CAR polypeptide of claim 1, wherein the TAA binding domains comprise an anti-CD33 binding domain and an anti-CLEC12A binding domain.

3. The bispecific CAR polypeptide of claim 1, wherein the bispecific CAR polypeptide is encoded by the nucleic acids sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

4. (canceled)

5. The bispecific CAR polypeptide of claim 1, wherein the TAA binding domains comprise an anti-CD33 binding domain and an anti-CD123 binding domain.

6. The bispecific CAR polypeptide of claim 1, wherein the TAA binding domains comprise an anti-CD33 binding domain and an anti-CD99 binding domain.

7. The bispecific CAR polypeptide of claim 1, wherein the TAA binding domains comprise an anti-CLEC12A binding domain and an anti-CD99 binding domain.

8. The bispecific CAR polypeptide of claim 1, wherein the TAA binding domains comprise an anti-CLEC12A binding domain and an anti-CD123 binding domain.

9. The bispecific CAR polypeptide of claim 1, wherein the TAA binding domains comprise an anti-CD123 binding domain and an anti-CD99 binding domain.

10. An isolated nucleic acid encoding the bispecific CAR polypeptide of claim 1.

11. A vector comprising the nucleic acid of claim 10.

12. (canceled)

13. (canceled)

14. (canceled)

15. (canceled)

16. (canceled)

17. (canceled)

18. (canceled)

19. (canceled)

20. (canceled)

21. (canceled)

22. A cell comprising the polypeptide of claim 1.

23. The cell of claim 22, wherein the cell is selected from the group consisting of a αβT cell, γδT cell, a natural killer (NK) cell, a natural killer T (NKT) cell, a macrophage, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

24. The cell of claim 22, wherein the cell exhibits an anti-tumor immunity when a TAA binding domain of said cell binds to a TAA of a cancer cell.

25. A method of treating a cancer in a subject in a subject in need thereof, the method comprising administering to the subject a therapeutically effective amount of the cells of claim 22.

26. (canceled)

27. (canceled)

28. (canceled)

29. (canceled)

30. (canceled)

31. (canceled)

32. (canceled)

33. (canceled)

34. (canceled)

35. The method of claim 25, wherein the cell is selected from the group consisting of a αβT cell, γδT cell, a natural killer (NK) cell, a natural killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

36. The method of claim 25, wherein the method further comprises administering an immunomodulating agent.

37. The method of claim 36, wherein the immunomodulating agent is a PD-1 antibody, a PD-L1 antibody, a CTLA-4 antibody, or any combination thereof.

38. The method of any one of claims 25-37, wherein the subject is a mammal.

39. The metho of any one of claim 38, wherein the subject is a human.


SEQUENCE LISTING (223 sequences)

SEQ ID NO: 1 - DNA, 1586 bp, synthetic construct
  misc_feature 1..1586: note = Description of sequence: anti-CD33-CLEC12A scFv1
  source 1..1586: mol_type = other DNA
ccatggccctcccggtaacggctctgctgcttccactcgcactgctcttgcatgctgccagaccacaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacggggtggaggtggatcaggtggaggtggatctggtggaggtggatctgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggaggaggaggtagcggaggaggcggaagcggtggtggaggctcccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcgggtccggcagcggcactagctactctttaaccatcagcagcatggagagcgaggacgctgctacctactactgtcagcagtggacctccaatccccctacctttggcggcggcaccaagctggagatcaaggcggccgcc

SEQ ID NO: 2 - DNA, 1580 bp, synthetic construct
  misc_feature 1..1580: note = Description of sequence: anti-CD33-CLEC12A scFv2
  source 1..1580: mol_type = other DNA
ccatggccctcccggtaacggctctgctgcttccactcgcactgctcttgcatgctgccagaccagaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacggggtggaggtggatcaggtggaggtggatctggtggaggtggatctgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggaggaggaggtagcggaggaggcggaagcggtggtggaggctcccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcgggtccggcagcggcactagctactctttaaccatcagcagcatggagagcgaggacgctgctacctactactgtcagcagtggacctccaatccccctacctttggcggcggcaccaagctggagatcaaggcggccgcc

SEQ ID NO: 3 - DNA, 1580 bp, synthetic construct
  misc_feature 1..1580: note = Description of sequence: anti-CD33-CLEC12A scFv3
  source 1..1580: mol_type = other DNA
ccatggctttacccgttacagctttactgctgcctctggctttattattacatgccgctagacccgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggaggaggaggtagcggaggaggcggaagcggtggtggaggctcccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcgggtccggcagcggcactagctactctttaaccatcagcagcatggagagcgaggacgctgctacctactactgtcagcagtggacctccaatccccctacctttggcggcggcaccaagctggagatcaagggtggaggtggatcaggtggaggtggatctggtggaggtggatctgaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgggcggccgca

SEQ ID NO: 4 - DNA, 1585 bp, synthetic construct
  misc_feature 1..1585: note = Description of sequence: anti-CD33-CLEC12A scFv4
  source 1..1585: mol_type = other DNA
ccatggctttacccgttacagctttactgctgcctctggctttattattacatgccgctagacccgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggaggaggaggtagcggaggaggcggaagcggtggtggaggctcccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcgggtccggcagcggcactagctactctttaaccatcagcagcatggagagcgaggacgctgctacctactactgtcagcagtggacctccaatccccctacctttggcggcggcaccaagctggagatcaagggtggaggtggatcaggtggaggtggatctggtggaggtggatctcaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgggcggccgc

SEQ ID NO: 5 - DNA, 1529 bp, synthetic construct
  misc_feature 1..1529: note = Description of sequence: anti-CD33-CLEC12A scFv5
  source 1..1529: mol_type = other DNA
ccatggccctcccggtaacggctctgctgcttccactcgcactgctcttgcatgctgccagaccagaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggtggaggtggatctgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggcagcaccagcggcagcggcaaaccgggcagcggcgaaggcagcaccaaaggccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcggatccggcagcggcactagctactctttaaccatcagcagcatggagagcgaggacgctgctacctactactgtcagcagtggacctccaatccccctacctttggcggcggcaccaagctggagatcaagggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgggcggccgca

SEQ ID NO: 6 - DNA, 1534 bp, synthetic construct
  misc_feature 1..1534: note = Description of sequence: anti-CD33-CLEC12A scFv6
  source 1..1534: mol_type = other DNA
ccatggccctcccggtaacggctctgctgcttccactcgcactgctcttgcatgctgccagaccacaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggtggaggtggatctgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggcagcaccagcggcagcggcaaaccgggcagcggcgaaggcagcaccaaaggccagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcggatccggcagcggcactagctactctttaaccatcagcagcatggagagcgaggacgctgctacctactactgtcagcagtggacctccaatccccctacctttggcggcggcaccaagctggagatcaagggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgggcggccgc

SEQ ID NO: 7 - DNA, 1535 bp, synthetic construct
  misc_feature 1..1535: note = Description of sequence: anti-CD33-CLEC12A scFv7
  source 1..1535: mol_type = other DNA
ccatggctttacccgttaccgctctgctgctgcctctggctctgctgctccatgctgctcgtcccgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggtggaggtggatctgaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggcagcaccagcggcagcggcaaaccgggcagcggcgaaggcagcaccaaaggcgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgggcgggtggaggtggatctcagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcggatccggcagcggcactagctactctttaaccatcagcagcatggagagcgaggacgctgctacctactactgtcagcagtggacctccaatccccctacctttggcggcggcaccaagctggagatcaagcgggcggccgcc

SEQ ID NO: 8 - DNA, 1538 bp, synthetic construct
  misc_feature 1..1538: note = Description of sequence: anti-CD33-CLEC12A scFv8
  source 1..1538: mol_type = other DNA
ccatggctttacccgttaccgctctgctgctgcctctggctctgctgctccatgctgctcgtcccgagctgattctggtggagtccggaggcggactggtgaagcccggtggctctttaaagctgagctgcgccgtgagcggcttcaccttcagctccttcgccatgagctgggttcgtcagacccccgaaaagaggctggaatgggtcgccaccatctccagcggcggcgcctacaccttctacaaggacagcgtgaagggtcgtttcacaatctctcgtgacaacgccaagaacaccctctatttacagatgagcagcttacgtagcgaggacagcgccatgtactactgcgccagacacagcggatacgacggctactacctctatgctatggattactggggccaaggtacctccgtgaccgtgagctctggtggaggtggatctcaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggcagcaccagcggcagcggcaaaccgggcagcggcgaaggcagcaccaaaggcgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacggggtggaggtggatctcagatcgtgctgacccagtcccccgagattatgagcgccagccccggcgagaaggtgaccatgacttgttccgcctcctcctccgtgcattacatgcactggtaccagcagaagtctggcaccagccccaagaggtggatctacgacacaagcaagctggctagcggagtgcccggaaggttcagcggatccggcagcggcactagctactctttaaccatcagcagcatggagagcgaggacgctgctacctactactgtcagcagtggacctccaatccccctacctttggcggcggcaccaagctggagatcaagcgggcggccgcc

SEQ ID NO: 9 - protein, 121 aa, synthetic construct
  REGION 1..121: note = Description of sequence: anti-CD123, 3F5HC1
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSS

SEQ ID NO: 10 - protein, 118 aa, synthetic construct
  REGION 1..118: note = Description of sequence: anti-CD123, 12H1HC1
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSS

SEQ ID NO: 11 - protein, 122 aa, synthetic construct
  REGION 1..122: note = Description of sequence: anti-CD 123, 12H1HC2
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSA

SEQ ID NO: 12 - protein, 121 aa, synthetic construct
  REGION 1..121: note = Description of sequence: anti-CD 123, 12H2HC1
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSS

SEQ ID NO: 13 - protein, 120 aa, synthetic construct
  REGION 1..120: note = Description of sequence: anti-CD 123, 5A12HC1
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSS

SEQ ID NO: 14 - protein, 119 aa, synthetic construct
  REGION 1..119: note = Description of sequence: anti-CD 123, 15A12HC2
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSS

SEQ ID NO: 15 - protein, 107 aa, synthetic construct
  REGION 1..107: note = Description of sequence: anti-CD 123, 3F5LC1
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 16 - protein, 113 aa, synthetic construct
  REGION 1..113: note = Description of sequence: anti-CD123, 12H1 LC1
DIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 17 - protein, 112 aa, synthetic construct
  REGION 1..112: note = Description of sequence: anti- CD 123, 12H2LC
DVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 18 - protein, 106 aa, synthetic construct
  REGION 1..106: note = Description of sequence: anti-CD 123, 15A12LC1
DIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 19 - protein, 112 aa, synthetic construct
  REGION 1..112: note = Description of sequence: anti-CD 123, 15A12LC2
DIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 20 - protein, 243 aa, synthetic construct
  REGION 1..243: note = Description of sequence: anti-CD 123, 3F5HC1_LC
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSSGGGGSGGGGSGGGGSDILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 21 - protein, 246 aa, synthetic construct
  REGION 1..246: note = Description of sequence: anti-CD 123, 12H1 HC1_LC1
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSSGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 22 - protein, 250 aa, synthetic construct
  REGION 1..250: note = Description of sequence: anti-CD 123, 12H1 HC2_LC1
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSAGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 23 - protein, 248 aa, synthetic construct
  REGION 1..248: note = Description of sequence: anti-CD 123, 12H2HC1_LC1
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSSGGGGSGGGGSGGGGSDVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 24 - protein, 241 aa, synthetic construct
  REGION 1..241: note = Description of sequence: anti-CD 123, 15A12HC1_LC1
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 25 - protein, 247 aa, synthetic construct
  REGION 1..247: note = Description of sequence: anti-CD 123, 15A12HC1_LC2
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 26 - protein, 240 aa, synthetic construct
  REGION 1..240: note = Description of sequence: anti-CD 123, 15A12HC2_LC1
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 27 - protein, 246 aa, synthetic construct
  REGION 1..246: note = Description of sequence: anti-CD 123, 15A12HC2_LC2
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 28 - DNA, 806 bp, synthetic construct
  misc_feature 1..806: note = Description of sequence: anti-CD 123, 3F5HC1_LC
  source 1..806: mol_type = other DNA
ccatggccctcccggtaacggctctgctgcttccactcgcactgctcttgcatgctgccagaccagaggtccagctgcaacagtctggacctgagctggtgaagcctgggtcttcagtgaagatatcctgcaaagcttctggatacacattcactgactacaacatggactgggtgaagcagagtcatggaaagagccttgagtggattggaactattaatcctaacaatggtggtactagctacaaccagaagttcaagggcaaggccacattgactgtagacaagtcctccagcacagcctacatggagctccgcagcctgacatctgaagactctgcagtctattactgtgcaagaaagggctatggtggtaactacgactactttgactactggggccaaggcaccactctcacagtctcctcaggtggaggtggatcaggtggaggtggatctggtggaggtggatctgacatcttgctgactcagtctccagccatcctgtctgtgagtccaggagaaagagtcagtttctcctgcagggccagtcagagcattggcacaagcatacactggtatcagcaaagaacaaatggttctccaaggcttctcataaagtatgcttctgagtctatctctgggttcccttccaggtttagtggcagtggatcagggacagattttactcttagcatcaacagtgtggagtctgaagatattgcagattattactgtcaacaaagtaatagctggccgtacacgttcggaggggggaccaagctggaaataaaacgggcggccgca

SEQ ID NO: 29 - DNA, 804 bp, synthetic construct
  misc_feature 1..804: note = Description of sequence: anti-CD 123, 12H1 HC1_LC1
  source 1..804: mol_type = other DNA
atggccctcccggtaacggctctgctgcttccactcgcactgctcttgcatgctgccagaccagaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactatatatgcctcaaagttccagggcaaggccactataacagcagacacatcatccaacacagcctacatgcagctcagcagcctgacatctggggacactgccgtctattactgtgctctttattactatggtggtagccttgactactggggccaaggcaccactctcacagtctcctcaggtggaggtggatcaggtggaggtggatctggtggaggtggatctgacattgtgatgtcacagtctccatcctccctagctgtgtcagttggagagagggttactatgagctgcaagtccagtcagagccttttatatagtggcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcggacgttcggtggaggcaccaagctggaaatcaaacgg

SEQ ID NO: 30 - DNA, 816 bp, synthetic construct
  misc_feature 1..816: note = Description of sequence: anti-CD 123, 12H1 HC1_LC2
  source 1..816: mol_type = other DNA
atggccctcccggtaacggctctgctgcttccactcgcactgctcttgcatgctgccagaccacaggttactctgaaagagtctggccctgggatattgcagccctcccagaccctcagtctgacttgttctttctctgggttttcactgagcacttatggtatgggtgtgagctggattcgtcagccttcaggaaagggtctggagtggctggcacacatttactgggatgatgacaagcgctataacccatccctgaagagccggctcacaatctccaaggatacctccaacaaccaggtattcctcaagatcaccagtgtggacactgcagatactgccacatactactgtgctcaaagcctgatctatgatggttactacgggtttgcctactggggccaagggactctggtcactgtctctgcaggtggaggtggatcaggtggaggtggatctggtggaggtggatctgacattgtgatgtcacagtctccatcctccctagctgtgtcagttggagagagggttactatgagctgcaagtccagtcagagccttttatatagtggcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcggacgttcggtggaggcaccaagctggaaatcaaacgg

SEQ ID NO: 31 - DNA, 808 bp, synthetic construct
  misc_feature 1..808: note = Description of sequence: anti-CD 123, 12H2HC1_LC1
  source 1..808: mol_type = other DNA
atggccctcccggtaacggctctgctgcttccactcgcactgctcttgcatgctgccagaccacagatccagttggtgcaatctggacctgagctgaagaagcctggagagacagtcaagatctcctgcaaggcttctgggtataccttcacatactatggaatgaactgggtgaagcaggctccaggaaagggtttagagtggatgggctggataaacacctactctggagtgccaacatatgctgatgacttcaagggacggtttgccttctctttggaaacctctgtcagcactgcctatttgcagatcaacaacctcaaaaatgaggacacggctacatatttttgtgcaagatggatctactatagtgacctctatggtatggactactggggtcaaggaacctcagtcaccgtctcctcaggtggaggtggatcaggtggaggtggatctggtggaggtggatctgatgttttgatgacccaaagtccactctccctgcctgtcagtcttggagatcaagcctccatctcttgtagatctagtcagagtattgtacatagtaatggagacacgtatttagaatggtatttgcagaaaccaggccagtctccaaagctcctgatctacaaagtttctaaccgattttctggggtcccagacaggttcagtggcagtggatcagggacagatttcacactcaagatcagcagagtggaggctgaggatctgggagtttatcactgctttcaaggttcacatgttccgtggacgttcggtggaggcaccaagctggaaatcaaac

SEQ ID NO: 32 - DNA, 789 bp, synthetic construct
  misc_feature 1..789: note = Description of sequence: anti-CD 123, 15A12HC1_LC1
  source 1..789: mol_type = other DNA
atggccctcccggtaacggctctgctgcttccactcgcactgctcttgcatgctgccagaccacaggtccagctgcagcagtctggggctgaactggcaaaacctggggcctcagtgaagatgtcctgcaaggcttctggctacaccttttctagctactggatgcactggctaaaacagaggcctggacagggtctggagtggattggatacattaatcctagcagtggttatactaactacaatcagaagttcaaggacaaggccacattgactgcagacaaatcctccagcacagcctacatgcaactgagcagcctgacatctgaggactctgcagtctattactgtgcaagagatggtaactatgaccactggtacttcgatgtctggggcacagggaccacggtcaccgtctcctcaggtggaggtggatcaggtggaggtggatctggtggaggtggatctgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagttgctcacgttcggtgctgggaccaagctggagctgaaacgg

SEQ ID NO: 33 - DNA, 807 bp, synthetic construct
  misc_feature 1..807: note = Description of sequence: anti-CD 123, 15A12HC1_LC2
  source 1..807: mol_type = other DNA
atggccctcccggtaacggctctgctgcttccactcgcactgctcttgcatgctgccagaccacaggtccagctgcagcagtctggggctgaactggcaaaacctggggcctcagtgaagatgtcctgcaaggcttctggctacaccttttctagctactggatgcactggctaaaacagaggcctggacagggtctggagtggattggatacattaatcctagcagtggttatactaactacaatcagaagttcaaggacaaggccacattgactgcagacaaatcctccagcacagcctacatgcaactgagcagcctgacatctgaggactctgcagtctattactgtgcaagagatggtaactatgaccactggtacttcgatgtctggggcacagggaccacggtcaccgtctcctcaggtggaggtggatcaggtggaggtggatctggtggaggtggatctgacattgtgatgtcacagtctccatcctccctggctgtgtcagcaggagagagggtcactatgagctgcagatccagtcagagtctgctcaacagtagaacccgaaagaactacttggcttggtaccagcagaaaccagggcagtctcctaagctgctgatctactgggcatccactagggaatctggggtccctgatcgcttctcaggcagtggatctgggacagatttcactctcaccatcagcagtgtgcaggctgaagacctggcagtttattactgcgagcaatcttataatctattcacgttcggctcggggacaaagttggaaataaaacgg

SEQ ID NO: 34 - DNA, 786 bp, synthetic construct
  misc_feature 1..786: note = Description of sequence: anti-CD 123, 15A12HC2_LC1
  source 1..786: mol_type = other DNA
atggccctcccggtaacggctctgctgcttccactcgcactgctcttgcatgctgccagaccacaggttcaactgcagcagcctggggctgagctggtgaggcctggggcttcagtgaagatgtcctgcaaggcttctggctacaccctcaccacctacttgatggactgggtaaaacagaggcttggacaaggctttgagtggattggaaatattaatcctaatagtggtagtagtaactacaatgagaagttcaagggcaaggccaagctgactgtagacaaatcctccagcacagcctacatgcaactcagcagcctgacatctgaggactctgcggtctattactgtgcaatacggcactatggtggtagtctctttgactactggggccaaggcaccactctcacagtctcctcaggtggaggtggatcaggtggaggtggatctggtggaggtggatctgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagttgctcacgttcggtgctgggaccaagctggagctgaaacgg

SEQ ID NO: 35 - DNA, 804 bp, synthetic construct
  misc_feature 1..804: note = Description of sequence: anti-CD 123, 15A12HC2_LC2
  source 1..804: mol_type = other DNA
atggccctcccggtaacggctctgctgcttccactcgcactgctcttgcatgctgccagaccacaggttcaactgcagcagcctggggctgagctggtgaggcctggggcttcagtgaagatgtcctgcaaggcttctggctacaccctcaccacctacttgatggactgggtaaaacagaggcttggacaaggctttgagtggattggaaatattaatcctaatagtggtagtagtaactacaatgagaagttcaagggcaaggccaagctgactgtagacaaatcctccagcacagcctacatgcaactcagcagcctgacatctgaggactctgcggtctattactgtgcaatacggcactatggtggtagtctctttgactactggggccaaggcaccactctcacagtctcctcaggtggaggtggatcaggtggaggtggatctggtggaggtggatctgacattgtgatgtcacagtctccatcctccctggctgtgtcagcaggagagagggtcactatgagctgcagatccagtcagagtctgctcaacagtagaacccgaaagaactacttggcttggtaccagcagaaaccagggcagtctcctaagctgctgatctactgggcatccactagggaatctggggtccctgatcgcttctcaggcagtggatctgggacagatttcactctcaccatcagcagtgtgcaggctgaagacctggcagtttattactgcgagcaatcttataatctattcacgttcggctcggggacaaagttggaaataaaacgg

SEQ ID NO: 36 - protein, 114 aa, synthetic construct
  REGION 1..114: note = Description of sequence: anti-CD99, 1 H3H7, 1 H3H9
EVQLQQSGAELVKPGASVKLSCTASGFDIKDTYIHVWKQRPEQGLEWIGRIDPANGDTRYDPEFQGKASLTADTSSNTAYLQFSNLTSEDTAVYYCARRGGLSWGQGTTLTVSS

SEQ ID NO: 37 - DNA, 342 bp, synthetic construct
  misc_feature 1..342: note = Description of sequence: anti-CD 99, 1 H3H7, 1 H3H9
  source 1..342: mol_type = other DNA
gaggttcaactgcaacagtctggggcagagcttgtgaagccaggggcctcagtcaagttgtcctgcacagcttctggcttcgacattaaagacacctatatccactgggtgaaacagaggcctgaacagggcctggagtggattggaaggattgatcctgcgaatggtgatactagatatgacccggaattccagggcaaggcctctctaacagctgacacatcctccaatacagcctacctacaattcagcaacctgacatctgaagacactgccgtctattactgtgctagaagaggcggcctctcctggggccaaggcaccactctcacagtctcctca

SEQ ID NO: 38 - protein, 116 aa, synthetic construct
  REGION 1..116: note = Description of sequence: anti-CD99, 4C5E2
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSS

SEQ ID NO: 39 - DNA, 348 bp, synthetic construct
  misc_feature 1..348: note = Description of sequence: anti-CD 99, 4C5E2
  source 1..348: mol_type = other DNA
gaggtgcagctggaggagtctggtggaggattggtgcagcctaaaggatcattgaaactctcatgtgccgcctccggtttcaccttcaatacctatgccatgtactgggtctgccaggctccaggaaagggtttgaaatgggttgctcgcataagaagtaaagttaataattatgcaacatattatgccgattcagtgaaagacagattcaccatctccagagatgattcacaaaacatgctctttctgcacatgaacaacctgaaaactgaggacactgccatatatttctgtgtgagagatcctatggactactggggtcaaggaatctcagtcaccgtctcctca

SEQ ID NO: 40 - protein, 116 aa, synthetic construct
  REGION 1..116: note = Description of sequence: anti-CD99, 4C5H10
EVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSS

SEQ ID NO: 41 - DNA, 348 bp, synthetic construct
  misc_feature 1..348: note = Description of sequence: anti-CD99, 4C5H10
  source 1..348: mol_type = other DNA
gaggtgcagcttgttgagtctggtggaggattggtgcagcctaaaggatcattgaaactctcatgtgccgcctccggtttcaccttcaatacctatgccatgtactgggtctgccaggctccaggaaagggtttgaaatgggttgctcgcataagaagtaaagttaataattatgcaacatattatgccgattcagtgaaagacagattcaccatctccagagatgattcacaaaacatgctctttctgcacatgaacaacctgaaaactgaggacactgccatatatttctgtgtgagagatcctatggactactggggtcaaggaatctcagtcaccgtctcctca

SEQ ID NO: 42 - protein, 121 aa, synthetic construct
  REGION 1..121: note = Description of sequence: anti-CD99, 9G12C9
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 43 - DNA, 363 bp, synthetic construct
  misc_feature 1..363: note = Description of sequence: anti-CD99, 9G12C9
  source 1..363: mol_type = other DNA
caggttcagctccagcagtctggggctgagctggcaagacctggggcttcagtgaagttgtcctgcaaggcttctggctacacgtttactactttctggatgcagtgggtaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 44 - protein, 121 aa, synthetic construct
  REGION 1..121: note = Description of sequence: anti-CD99, 9G12G6 HB1
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 45 - DNA, 363 bp, synthetic construct
  misc_feature 1..363: note = Description of sequence: anti-CD99, 9G12G6 HB1
  source 1..363: mol_type = other DNA
gatgtgaagcttcaggagtctggggctgagctggcaagacctggggcttcagtgaagttgtcctgcaaggcttctggctacacgtttactactttctggatgcagcgggtaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 46 - protein, 121 aa, synthetic construct
  REGION 1..121: note = Description of sequence: anti-CD99, 9G12G6 HB3
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 47 - DNA, 363 bp, synthetic construct
  misc_feature 1..363: note = Description of sequence: anti-CD99, 9G12G6 HB3
  source 1..363: mol_type = other DNA
caggtgcagctgaaggagtctggggctgagctggcaagacctggggcttcagtgaagttgtcctgcaaggcttctggctacacgtttactactttctggatgcagtgggcaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 48 - protein, 107 aa, synthetic construct
  REGION 1..107: note = Description of sequence: anti-CD99, 1 H3H9
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 49 - DNA, 321 bp, synthetic construct
  misc_feature 1..321: note = Description of sequence: anti-CD99, 1 H3H9
  source 1..321: mol_type = other DNA
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaa

SEQ ID NO: 50 - protein, 112 aa, synthetic construct
  REGION 1..112: note = Description of sequence: anti-CD99, 1 H3H7 LC1
GNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 51 - protein, 112 aa, synthetic construct
  REGION 1..112: note = Description of sequence: anti-CD99, 1 H3H7 LC2
GNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 52 - protein, 112 aa, synthetic construct
  REGION 1..112: note = Description of sequence: anti-CD99, 4C5E2
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 53 - DNA, 336 bp, synthetic construct
  misc_feature 1..336: note = Description of sequence: anti-CD99, 4C5E2
  source 1..336: mol_type = other DNA
gatattgtgatgactcaggctgcaccctctgtacctgtcactcctggggagtcagtatccatctcctgcgggtctagtaagagtctcctgcatagtaatggcaacacttacttgtattggttcctgcagaggccaggccagtctcctcagctcctgatatatcgggtgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaggctggaaataaaa

SEQ ID NO: 54 - protein, 113 aa, synthetic construct
  REGION 1..113: note = Description of sequence: anti-CD99, 9G12C9
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 55 - DNA, 339 bp, synthetic construct
  misc_feature 1..339: note = Description of sequence: anti-CD99, 9G12C9
  source 1..339: mol_type = other DNA
gacactgtgatgtcacagtccccatcctccctagctgtttcagttggagagaagataactatgagctgcaagtccagtcagagtcttttatgtcgtagcaatcaaaagaactacttggcctggtaccagcagaaaccagggcagtctcctaaacagctgatttactgggcatctactagggaatctggggtccctgatcgcttcacaggcagtggatctgggacagatttcactctcaccatcagcagtgtgaaggctgaagacctggcagtttattactgtcagcaatattatagttatccgctcacgttcggtgctggcaccaagctggagctgaaa

SEQ ID NO: 56 - protein, 113 aa, synthetic construct
  REGION 1..113: note = Description of sequence: anti-CD99, 9G12G6
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 57 - DNA, 339 bp, synthetic construct
  misc_feature 1..339: note = Description of sequence: anti-CD99, 9G12G6
  source 1..339: mol_type = other DNA
gacactgtgatgtcacagtccccatcctccctagctgtttcagttggagagaagataactatgagctgcaagtccagtcagagtcttttatatcgtagcaatcaaaagaactacttggcctggtaccagcagaaaccagggcagtctcctaaacagctgatttactgggcatctactagggaatctggggtccctgatcgcttcacaggcagtggatctgggacagatttcactctcaccatcagcagtgtgaaggctgaagacctggcagtttattactgtcagcaatattatagttatccgctcacgttcggtgctggcaccaagctggagctgaaa

SEQ ID NO: 58 - protein, 236 aa, synthetic construct
  REGION 1..236: note = Description of sequence: anti-CD99, 1 H3H9 v1
EVQLQQSGAELVKPGASVKLSCTASGFDIKDTYIHVWKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 59 - protein, 241 aa, synthetic construct
  REGION 1..241: note = Description of sequence: anti-CD99 scFv
EVQLQQSGAELVKPGASVKLSCTASGFDIKDTYIHVWKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 60 - protein, 238 aa, synthetic construct
  REGION 1..238: note = Description of sequence: anti-CD99 scFv
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 61 - protein, 243 aa, synthetic construct
  REGION 1..243: note = Description of sequence: anti-CD99 scFv
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 62 - protein, 243 aa, synthetic construct
  REGION 1..243: note = Description of sequence: anti-CD99, 4C5E2 v1
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 63 - protein, 244 aa, synthetic construct
  REGION 1..244: note = Description of sequence: anti-CD99 scFv
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 64 - protein, 244 aa, synthetic construct
  REGION 1..244: note = Description of sequence: anti-CD99 scFv
EVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 65 - protein, 238 aa, synthetic construct
  REGION 1..238: note = Description of sequence: anti-CD99 scFv
EVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 66 - protein, 243 aa, synthetic construct
  REGION 1..243: note = Description of sequence: anti-CD99 scFv
EVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 67 - protein, 243 aa, synthetic construct
  REGION 1..243: note = Description of sequence: anti-CD99, 4C5H10 v1)
EVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 68 - protein, 244 aa, synthetic construct
  REGION 1..244: note = Description of sequence: anti-CD99 scFv
EVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 69 - protein, 243 aa, synthetic construct
  REGION 1..243: note = Description of sequence: anti-CD99 scFv
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 70 - protein, 248 aa, synthetic construct
  REGION 1..248: note = Description of sequence: anti-CD99 scFv
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 71 - protein, 248 aa, synthetic construct
  REGION 1..248: note = Description of sequence: anti-CD99 scFv
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 72 - protein, 249 aa, synthetic construct
  REGION 1..249: note = Description of sequence: anti-CD99, 9G12C9 v1
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 73 - protein, 249 aa, synthetic construct
  REGION 1..249: note = Description of sequence: anti-CD99 scFv
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 74 - protein, 243 aa, synthetic construct
  REGION 1..243: note = Description of sequence: anti-CD99 scFv
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 75 - protein, 248 aa, synthetic construct
  REGION 1..248: note = Description of sequence: anti-CD99 scFv
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 76 - protein, 248 aa, synthetic construct
  REGION 1..248: note = Description of sequence: anti-CD99 scFv
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 77 - protein, 249 aa, synthetic construct
  REGION 1..249: note = Description of sequence: anti-CD99 scFv
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 78 - protein, 243 aa, synthetic construct
  REGION 1..243: note = Description of sequence: anti-CD scFv
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 79 - protein, 248 aa, synthetic construct
  REGION 1..248: note = Description of sequence: anti-CD99 scFv
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 80 - protein, 248 aa, synthetic construct
  REGION 1..248: note = Description of sequence: anti-CD99 scFv
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 81 - protein, 249 aa, synthetic construct
  REGION 1..249: note = Description of sequence: anti-CD99 scFv
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 82 - protein, 249 aa, synthetic construct
  REGION 1..249: note = Description of sequence: anti-CD99 scFv
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 83 - protein, 236 aa, synthetic construct
  REGION 1..236: note = Description of sequence: anti-CD99, 1 H3H9 v2
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLQQSGAELVKPGASVKLSCTASGFDIKDTYIHVWKQRPEQGLEWIGRIDPANGDTRYDPEFQGKASLTADTSSNTAYLQFSNLTSEDTAVYYCARRGGLSWGQGTTLTVSS

SEQ ID NO: 84 - protein, 238 aa, synthetic construct
  REGION 1..238: note = Description of sequence: anti-CD99 scFv
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLEESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSS

SEQ ID NO: 85 - protein, 238 aa, synthetic construct
  REGION 1..238: note = Description of sequence: antiCD99 scFv
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLVESGGGLVQPKGSLKLSCAASGFTFNTYAMYVWCQAPGKGLKVWARIRSKVNNYATYYADSVKDRFTISRDDSQNMLFLHMNNLKTEDTAIYFCVRDPMDYWGQGISVTVSS

SEQ ID NO: 86 - protein, 243 aa, synthetic construct
  REGION 1..243: note = Description of sequence: antiCD99 scFv
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 87 - protein, 243 aa, synthetic construct
  REGION 1..243: note = Description of sequence: anti-CD99 scFv
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 88 - protein, 243 aa, synthetic construct
  REGION 1..243: note = Description of sequence: anti-CD99 scFv
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 89 - protein, 241 aa, synthetic construct
  REGION 1..241: note = Description of sequence: anti-CD99 scFv
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 90 - protein, 243 aa, synthetic construct
  REGION 1..243: note = Description of sequence: anti-CD99, 4C5E2 M2
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 91 - protein, 243 aa, synthetic construct
  REGION 1..243: note = Description of sequence: anti-CD99, 4C5H10 M2
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 92 - protein, 248 aa, synthetic construct
  REGION 1..248: note = Description of sequence: anti-CD99 scFv
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLQQSGAELARPGASVKLSCKASGYTFTTFWMQVWKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 93 - protein, 248 aa, synthetic construct
  REGION 1..248: note = Description of sequence: anti-CD99 scFv
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 94 - protein, 248 aa, synthetic construct
  REGION 1..248: note = Description of sequence: anti-CD99 scFv
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 95 - protein, 242 aa, synthetic construct
  REGION 1..242: note = Description of sequence: anti-CD99 scFv
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 96 - protein, 244 aa, synthetic construct
  REGION 1..244: note = Description of sequence: anti-CD99 scFv
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 97 - protein, 249 aa, synthetic construct
  REGION 1..249: note = Description of sequence: anti-CD99, 9G12C9 M2
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 98 - protein, 249 aa, synthetic construct
  REGION 1..249: note = Description of sequence: anti-CD99 scFv
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 99 - protein, 249 aa, synthetic construct
  REGION 1..249: note = Description of sequence: anti-CD99 scFv
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 100 - protein, 242 aa, synthetic construct
  REGION 1..242: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 101 - protein, 244 aa, synthetic construct
  REGION 1..244: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 102 - protein, 244 aa, synthetic construct
  REGION 1..244: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKVWARIRSKVNNYATYYADSVKDRFTISRDDSQNMLFLHMNNLKTEDTAIYFCVRDPMDYWGQGISVTVSS

SEQ ID NO: 103 - protein, 249 aa, synthetic construct
  REGION 1..249: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 104 - protein, 249 aa, synthetic construct
  REGION 1..249: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 105 - protein, 249 aa, synthetic construct
  REGION 1..249: note = Description of sequence: anti-CD99 scFv
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 106 - protein, 123 aa, synthetic construct
  REGION 1..123: note = Description of sequence: anti-CLEC12A, 1 F3H8
ELILVESGGGLVKPGGSLKLSCAVSGFTFSSFAMSVWRQTPEKRLEVWATISSGGAYTFYKDSVKGRFTISRDNAKNTLYLQMSSLRSEDSAMYYCARHSGYDGYYLYAMDYWGQGTSVTVSS

SEQ ID NO: 107 - DNA, 369 bp, synthetic construct
  misc_feature 1..369: note = Description of sequence: anti-CLEC12A, 1 F3H8
  source 1..369: mol_type = other DNA
gaactaatactggtggagtctgggggaggcttagtgaagcctggagggtccctgaaactctcctgtgcagtctctggattcactttcagttcctttgccatgtcttgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 108 - protein, 127 aa, synthetic construct
  REGION 1..127: note = Description of sequence: anti-CLEC12A, 1 F3A10
GVQCELILVESGGGLVKPGGSLKLSCAVSGFTFSSFAVSVWRQTPEKRLEVWATISSGGAYTFYKDSVKGRFTISRDNAKNTLYLQMSSLRSEDSAMYYCARHSGYDGYYLYAMDYWGQGTSVTVSS

SEQ ID NO: 109 - DNA, 381 bp, synthetic construct
  misc_feature 1..381: note = Description of sequence: anti-CLEC12A, 1 F3A10
  source 1..381: mol_type = other DNA
ggtgtccagtgtgaactaatactggtggagtctgggggaggcttagtgaagcctggagggtccctgaaactctcctgtgcagtctctggattcactttcagttcctttgccgtgtcctgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 110 - protein, 121 aa, synthetic construct
  REGION 1..121: note = Description of sequence: anti-CLEC12A, 1 F3F3
EVQLEESGGGLVQPGGSLKVSCAVSGLAFSSHDMSVWRQTPEKRLEVWAYISGGGTNIYYSDTVKGRFTISRDNAKNTLYLQMSSLKSEDTAIYYCARPNYNYGGSWFAYWGQGTLVTVSA

SEQ ID NO: 111 - DNA, 363 bp, synthetic construct
  misc_feature 1..363: note = Description of sequence: anti-CLEC12A, 1 F3F3
  source 1..363: mol_type = other DNA
gaggtgcagctggaggagtctgggggaggcttagtgcagccgggagggtccctgaaagtctcctgtgcagtttccggactcgctttcagcagccatgacatgtcttgggttcgccagactccggagaagcggctggagtgggtcgcatacattagtggaggtggtactaatatctattattcagacactgtgaagggccgattcaccatctccagagacaatgccaagaacaccctgtacctgcaaatgagcagtctgaagtctgaagacacagccatttattactgtgcaagacccaattataattacggcggttcctggtttgcttactggggccaagggactctggtcactgtctctgca

SEQ ID NO: 112 - protein, 106 aa, synthetic construct
  REGION 1..106: note = Description of sequence: anti-CLEC12A, 1 F3H8, 1 F3F3, 1 F3A1 G
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 113 - DNA, 320 bp, synthetic construct
  misc_feature 1..320: note = Description of sequence: anti-CLEC12A, 1 F3H8, 1 F3F3, 1 F3A1 Q
  source 1..320: mol_type = other DNA
caaattgttctcacccagtctccagaaatcatgtctgcatctccaggggagaaggtcaccatgacctgcagtgccagctcaagtgtacattacatgcactggtaccagcagaagtcaggcacctcccccaaaagatggatttatgacacatccaaactggcttctggagtccctggtcgcttcagtggcagtgggtctgggacctcttactctctcacaatcagcagcatggagtctgaagatgctgccacttattactgccagcagtggactagtaacccacccacgttcggaggggggaccaagctggaaattaaacg

SEQ ID NO: 114 - protein, 244 aa, synthetic construct
  REGION 1..244: note = Description of sequence: anti-CLEC12A, 1 F3H8 v1
ELILVESGGGLVKPGGSLKLSCAVSGFTFSSFAMSVWRQTPEKRLEVWATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 115 - protein, 248 aa, synthetic construct
  REGION 1..248: note = Description of sequence: anti-CLEC12A, 1 F3A10 v1
GVQCELILVESGGGLVKPGGSLKLSCAVSGFTFSSFAVSVWRQTPEKRLEVWATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 116 - protein, 242 aa, synthetic construct
  REGION 1..242: note = Description of sequence: anti-CLEC12A, 1 F3F3 v1
EVQLEESGGGLVQPGGSLKVSCAVSGLAFSSHDMSVWRQTPEKRLEVWAYISGGGTNIYYSDTVKGRFTISRDNAKNTLYLQMSSLKSEDTAIYYCARPNYNYGGSWFAYWGQGTLVTVSAGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 117 - protein, 244 aa, synthetic construct
  REGION 1..244: note = Description of sequence: anti-CLEC12A, 1 F3H8 v2
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSELILVESGGGLVKPGGSLKLSCAVSGFTFSSFAMSVWRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 118 - protein, 248 aa, synthetic construct
  REGION 1..248: note = Description of sequence: anti-CLEC12A, 1 F3A10 v2
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSGVQCELILVESGGGLVKPGGSLKLSCAVSGFTFSSFAVSVWRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 119 - protein, 242 aa, synthetic construct
  REGION 1..242: note = Description of sequence: anti-CLEC12A, 1 F3F3 v2
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSEVQLEESGGGLVQPGGSLKVSCAVSGLAFSSHDMSWVRQTPEKRLEVWAYISGGGTNIYYSDTVKGRFTISRDNAKNTLYLQMSSLKSEDTAIYYCARPNYNYGGSWFAYWGQGTLVTVSA

SEQ ID NO: 120 - protein, 120 aa, synthetic construct
  REGION 1..120: note = Description of sequence: anti-CD33 scFv VH domain
EVKLVESGGGLVKPGASLKLSCAASGFTFSNYGMSVNRQTSDKRLEVWASISSGGGDTYYPDNVKGRFTISRENAKNTLYLQMSSLNSEDTALYYCARDYGGTWDYFDYWGQGTTLTVSS

SEQ ID NO: 121 - protein, 122 aa, synthetic construct
  REGION 1..122: note = Description of sequence: anti-CD33 scFv VH domain
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSS

SEQ ID NO: 122 - protein, 117 aa, synthetic construct
  REGION 1..117: note = Description of sequence: anti-CD33 scFv VH domain
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSS

SEQ ID NO: 123 - protein, 107 aa, synthetic construct
  REGION 1..107: note = Description of sequence: anti-CD33 scFv VL domain
DIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 124 - protein, 107 aa, synthetic construct
  REGION 1..107: note = Description of sequence: anti-CD33 scFv VL domain
SIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 125 - protein, 106 aa, synthetic construct
  REGION 1..106: note = Description of sequence: anti-CD33 scFv VL domain
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 126 - protein, 107 aa, synthetic construct
  REGION 1..107: note = Description of sequence: anti-CD33 scFv VL domain
DIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 127 - protein, 242 aa, synthetic construct
  REGION 1..242: note = Description of sequence: anti-CD33 6A11HC1_LC
EVKLVESGGGLVKPGASLKLSCAASGFTFSNYGMSVNRQTSDKRLEVWASISSGGGDTYYPDNVKGRFTISRENAKNTLYLQMSSLNSEDTALYYCARDYGGTWDYFDYWGQGTTLTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 128 - protein, 244 aa, synthetic construct
  REGION 1..244: note = Description of sequence: anti-CD33 6A11HC2_LC
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 129 - protein, 239 aa, synthetic construct
  REGION 1..239: note = Description of sequence: anti-CD33 27A3HCLC1
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSSIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 130 - protein, 237 aa, synthetic construct
  REGION 1..237: note = Description of sequence: anti-CD33 27A3HCLC2
QVQLKESGPGLVAPSQSLSITCTVSGFSLSRYSVHVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSQIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 131 - protein, 239 aa, synthetic construct
  REGION 1..239: note = Description of sequence: anti-CD33 27A3HCLC3
QVQLKESGPGLVAPSQSLSITCTVSGFSLSRYSVHVWRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 132 - DNA, 792 bp, synthetic construct
  misc_feature 1..792: note = Description of sequence: anti-CD33 6A11HC1_LC
  source 1..792: mol_type = other DNA
atggccctcccggtaacggctctgctgcttccactcgcactgctcttgcatgctgccagaccagaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgg

SEQ ID NO: 133 - DNA, 798 bp, synthetic construct
  misc_feature 1..798: note = Description of sequence: anti-CD33 6A11HC2_LC
  source 1..798: mol_type = other DNA
atggccctcccggtaacggctctgctgcttccactcgcactgctcttgcatgctgccagaccacaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgg

SEQ ID NO: 134 - DNA, 783 bp, synthetic construct
  misc_feature 1..783: note = Description of sequence: anti-CD33 27A3HC_LCl
  source 1..783: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctagtattgtgatgacccagactcccaaattcctgcttgtatcagcaggagacagggttaccataacctgcaaggccagtcagactgtgaatgatgatgtagcttggtatcaacagaagccaggacagtctcctaaattgctgatatattatgtatccaatcgccacactggagtccctgatcgcttcactggcagtggatatgggacggatttcactttcaccatcagcactgtgcaggctgaagacctggcagtttatttctgtcagcaggattatagctctccgtacacgttcggaggggggaccaagctggaaataaaacgg

SEQ ID NO: 135 - DNA, 780 bp, synthetic construct
  misc_feature 1..780: note = Description of sequence: anti-CD33 27A3HCLC2
  source 1..780: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccactcacgttcggtgctgggaccaagctggagctgaaacgg

SEQ ID NO: 136 - DNA, 783 bp, synthetic construct
  misc_feature 1..783: note = Description of sequence: anti-CD33 27A3HCLC3
  source 1..783: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctgacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagtctgcagcctgaagattttgggagttattactgtcaacatcattatggtactccgtacacgttcggaggggggaccaagctggaaataaaacgg

SEQ ID NO: 137 - protein, 8 aa, synthetic construct
  REGION 1..8: note = Description of sequence: anti-CD33 CDR1 VH domain
GFTFSNYG

SEQ ID NO: 138 - protein, 8 aa, synthetic construct
  REGION 1..8: note = Description of sequence: anti-CD33 CDR1 VH domain
GYTFTSYW

SEQ ID NO: 139 - protein, 8 aa, synthetic construct
  REGION 1..8: note = Description of sequence: anti-CD33 CDR1 VH domain
GFSLSRYS

SEQ ID NO: 140 - protein, 8 aa, synthetic construct
  REGION 1..8: note = Description of sequence: anti-CD33 CDR2 VH domain
ISSGGGDT

SEQ ID NO: 141 - protein, 7 aa, synthetic construct
  REGION 1..7: note = Description of sequence: anti-CD33 CDR2 VH domain
HPSDSET

SEQ ID NO: 142 - protein, 7 aa, synthetic construct
  REGION 1..7: note = Description of sequence: anti-CD33 CDR2 VH domain
IWGGGYT

SEQ ID NO: 143 - protein, 13 aa, synthetic construct
  REGION 1..13: note = Description of sequence: anti-CD33 CDR3 VH domain
ARDYGGTWDYFDY

SEQ ID NO: 144 - protein, 15 aa, synthetic construct
  REGION 1..15: note = Description of sequence: anti-CD33 CDR3 VH domain
AREEGQLGHGGAMDY

SEQ ID NO: 145 - protein, 11 aa, synthetic construct
  REGION 1..11: note = Description of sequence: anti-CD33 CDR3 VH domain
ARYIDSSGYDY

SEQ ID NO: 146 - protein, 6 aa, synthetic construct
  REGION 1..6: note = Description of sequence: anti-CD33 CDR1 VL domain
QDISKY

SEQ ID NO: 147 - protein, 6 aa, synthetic construct
  REGION 1..6: note = Description of sequence: anti-CD33 CDR1 VL domain
QTVNDD

SEQ ID NO: 148 - protein, 5 aa, synthetic construct
  REGION 1..5: note = Description of sequence: anti-CD33 CDR1 VL domain
SSVSY

SEQ ID NO: 149 - protein, 6 aa, synthetic construct
  REGION 1..6: note = Description of sequence: anti-CD33 CDR1 VL domain
ENIYSY

SEQ ID NO: 150-153 - skipped (4 entries)

SEQ ID NO: 154 - protein, 9 aa, synthetic construct
  REGION 1..9: note = Description of sequence: anti-CD33 CDR3 VL domain
QQGDTFPWT

SEQ ID NO: 155 - protein, 9 aa, synthetic construct
  REGION 1..9: note = Description of sequence: anti-CD33 CDR3 VL domain
QQDYSSPYT

SEQ ID NO: 156 - protein, 9 aa, synthetic construct
  REGION 1..9: note = Description of sequence: anti-CD33 CDR3 VL domain
QQWSSNPLT

SEQ ID NO: 157 - protein, 9 aa, synthetic construct
  REGION 1..9: note = Description of sequence: anti-CD33 CDR3 VL domain
QHHYGTPYT

SEQ ID NO: 158 - protein, 8 aa, synthetic construct
  REGION 1..8: note = Description of sequence: anti-CD123 scFv CDR1 VH domain
GYTFTDYN

SEQ ID NO: 159 - protein, 8 aa, synthetic construct
  REGION 1..8: note = Description of sequence: anti-CD123 scFv CDR2 VH domain
INPNNGGT

SEQ ID NO: 160 - protein, 14 aa, synthetic construct
  REGION 1..14: note = Description of sequence: anti-CD123 scFv CDR3 VH domain
ARKGYGGNYDYFDY

SEQ ID NO: 161 - protein, 6 aa, synthetic construct
  REGION 1..6: note = Description of sequence: anti-CD123 scFv CDR1 VL domain
QSIGTS

SEQ ID NO: 162 - skipped

SEQ ID NO: 163 - protein, 9 aa, synthetic construct
  REGION 1..9: note = Description of sequence: anti-CD123 scFv CDR3 VL domain
QQSNSWPYT

SEQ ID NO: 164 - protein, 8 aa, synthetic construct
  REGION 1..8: note = Description of sequence: anti-CD123 scFv CDR1 VH domain
GFNIKDTY

SEQ ID NO: 165 - protein, 10 aa, synthetic construct
  REGION 1..10: note = Description of sequence: anti-CD123 scFv CDR1 VH domain
GFSLSTYGMG

SEQ ID NO: 166 - protein, 8 aa, synthetic construct
  REGION 1..8: note = Description of sequence: anti-CD123 scFv CDR2 VH domain
IDPANGNT

SEQ ID NO: 167 - protein, 7 aa, synthetic construct
  REGION 1..7: note = Description of sequence: anti-CD123 scFv CDR2 VH domain
LYWDDDK

SEQ ID NO: 168 - protein, 11 aa, synthetic construct
  REGION 1..11: note = Description of sequence: anti-CD123 scFv CDR3 VH domain
ALYYYGGSLDY

SEQ ID NO: 169 - protein, 14 aa, synthetic construct
  REGION 1..14: note = Description of sequence: anti-CD123 scFv CDR3 VH domain
AQSLIYDGYYGFAY

SEQ ID NO: 170 - protein, 12 aa, synthetic construct
  REGION 1..12: note = Description of sequence: anti-CD123 scFv CDR1 VL domain
QSLLYSGNQKNY

SEQ ID NO: 171 - skipped

SEQ ID NO: 172 - protein, 9 aa, synthetic construct
  REGION 1..9: note = Description of sequence: anti-CD123 scFv CDR3 VL domain
QQYYSYPRT

SEQ ID NO: 173 - protein, 8 aa, synthetic construct
  REGION 1..8: note = Description of sequence: anti-CD123 scFv CDR1 VH domain
GYTFTYYG

SEQ ID NO: 174 - protein, 8 aa, synthetic construct
  REGION 1..8: note = Description of sequence: anti-CD123 scFv CDR2 VH domain
INTYSGVP

SEQ ID NO: 175 - protein, 14 aa, synthetic construct
  REGION 1..14: note = Description of sequence: anti-CD123 scFv CDR3 VH domain
ARWIYYSDLYGMDY

SEQ ID NO: 176 - protein, 11 aa, synthetic construct
  REGION 1..11: note = Description of sequence: anti-CD123 scFv CDR1 VL domain
QSIVHSNGDTY

SEQ ID NO: 177 - skipped

SEQ ID NO: 178 - protein, 9 aa, synthetic construct
  REGION 1..9: note = Description of sequence: anti-CD123 scFv CDR3 VL domain
FQGSHVPWT

SEQ ID NO: 179 - protein, 8 aa, synthetic construct
  REGION 1..8: note = Description of sequence: anti-CD123 scFv CDR1 VH domain
GYTFSSYW

SEQ ID NO: 180 - protein, 8 aa, synthetic construct
  REGION 1..8: note = Description of sequence: anti-CD123 scFv CDR1 VH domain
GYTLTTYL

SEQ ID NO: 181 - protein, 8 aa, synthetic construct
  REGION 1..8: note = Description of sequence: anti-CD123 scFv CDR2 VH domain
INPSSGYT

SEQ ID NO: 182 - protein, 8 aa, synthetic construct
  REGION 1..8: note = Description of sequence: anti-CD123 scFv CDR2 VH domain
INPNSGSS

SEQ ID NO: 183 - protein, 13 aa, synthetic construct
  REGION 1..13: note = Description of sequence: anti-CD123 scFv CDR3 VH domain
ARDGNYDHWYFDV

SEQ ID NO: 184 - protein, 12 aa, synthetic construct
  REGION 1..12: note = Description of sequence: anti-CD123 scFv CDR3 VH domain
AIRHYGGSLFDY

SEQ ID NO: 185 - protein, 6 aa, synthetic construct
  REGION 1..6: note = Description of sequence: anti-CD123 scFv CDR1 VL domain
QDINSY

SEQ ID NO: 186 - protein, 12 aa, synthetic construct
  REGION 1..12: note = Description of sequence: anti-CD123 scFv CDR1 VL domain
QSLLNSRTRKNY

SEQ ID NO: 187 - skipped

SEQ ID NO: 188 - protein, 8 aa, synthetic construct
  REGION 1..8: note = Description of sequence: anti-CD123 scFv CDR3 VL domain
LQYDELLT

SEQ ID NO: 189 - protein, 8 aa, synthetic construct
  REGION 1..8: note = Description of sequence: anti-CD123 scFv CDR3 VL domain
EQSYNLFT

SEQ ID NO: 190 - protein, 8 aa, synthetic construct
  REGION 1..8: note = Description of sequence: anti-CD99 scFv CDR1 VH domain
GFDIKDTY

SEQ ID NO: 191 - protein, 5 aa, synthetic construct
  REGION 1..5: note = Description of sequence: anti-CD99 scFv CDR1 VH domain
TYAMY

SEQ ID NO: 192 - protein, 4 aa, synthetic construct
  REGION 1..4: note = Description of sequence: anti-CD99 scFv CDR1 VH domain
TFWM

SEQ ID NO: 193 - protein, 5 aa, synthetic construct
  REGION 1..5: note = Description of sequence: anti-CD99 scFv CDR1 VH domain
TFWMQ

SEQ ID NO: 194 - protein, 8 aa, synthetic construct
  REGION 1..8: note = Description of sequence: anti-CD99 scFv CDR2 VH domain
IDPANGDT

SEQ ID NO: 195 - protein, 22 aa, synthetic construct
  REGION 1..22: note = Description of sequence: anti-CD99 scFv CDR2 VH domain
RIRSKVNNYATYYADSVKDRFT

SEQ ID NO: 196 - protein, 20 aa, synthetic construct
  REGION 1..20: note = Description of sequence: anti-CD99 scFv CDR2 VH domain
TIYPGDDDTRYTQKFKGRAT

SEQ ID NO: 197 - protein, 7 aa, synthetic construct
  REGION 1..7: note = Description of sequence: anti-CD99 scFv CDR3 VH domain
ARRGGLS

SEQ ID NO: 198 - protein, 5 aa, synthetic construct
  REGION 1..5: note = Description of sequence: anti-CD99 scFv CDR3 VH domain
DPMDY

SEQ ID NO: 199 - protein, 12 aa, synthetic construct
  REGION 1..12: note = Description of sequence: anti-CD99 scFv CDR3 VH domain
SGYERGPYYFDS

SEQ ID NO: 200 - protein, 10 aa, synthetic construct
  REGION 1..10: note = Description of sequence: anti-CD99 scFv CDR3 VH domain
SGYERGPYYF

SEQ ID NO: 201 - protein, 6 aa, synthetic construct
  REGION 1..6: note = Description of sequence: anti-CD99 scFv CDR1 VL domain
GNIHNY

SEQ ID NO: 202 - protein, 16 aa, synthetic construct
  REGION 1..16: note = Description of sequence: anti-CD99 scFv CDR1 VL domain
GSSKSLLHSNGNTYLY

SEQ ID NO: 203 - protein, 17 aa, synthetic construct
  REGION 1..17: note = Description of sequence: anti-CD99 scFv CDR1 VL domain
KSSQSLLCRSNQKNYLA

SEQ ID NO: 204 - protein, 17 aa, synthetic construct
  REGION 1..17: note = Description of sequence: anti-CD99 scFv CDR1 VL domain
KSSQSLLYRSNQKNYLA

SEQ ID NO: 205 - protein, 7 aa, synthetic construct
  REGION 1..7: note = Description of sequence: anti-CD99 scFv CDR2 VL domain
RVSNLAS

SEQ ID NO: 206 - protein, 7 aa, synthetic construct
  REGION 1..7: note = Description of sequence: anti-CD99 scFv CDR2 VL domain
WASTRES

SEQ ID NO: 207 - protein, 9 aa, synthetic construct
  REGION 1..9: note = Description of sequence: anti-CD99 scFv CDR3 VL domain
QHFWSTPWT

SEQ ID NO: 208 - protein, 9 aa, synthetic construct
  REGION 1..9: note = Description of sequence: anti-CD99 scFv CDR3 VL domain
MQHLEYPYT

SEQ ID NO: 209 - protein, 9 aa, synthetic construct
  REGION 1..9: note = Description of sequence: anti-CD99 scFv CDR3 VL domain
QQYYSYPLT

SEQ ID NO: 210 - protein, 8 aa, synthetic construct
  REGION 1..8: note = Description of sequence: anti-CLEC12A CDR1 VH domain
GFTFSSFA

SEQ ID NO: 211 - protein, 5 aa, synthetic construct
  REGION 1..5: note = Description of sequence: anti-CLEC12A CDR1 VH domain
SFAVS

SEQ ID NO: 212 - protein, 5 aa, synthetic construct
  REGION 1..5: note = Description of sequence: anti-CLEC12A CDR1 VH domain
SHDMS

SEQ ID NO: 213 - protein, 8 aa, synthetic construct
  REGION 1..8: note = Description of sequence: anti-CLEC12A CDR2 VH domain
ISSGGAYT

SEQ ID NO: 214 - protein, 20 aa, synthetic construct
  REGION 1..20: note = Description of sequence: anti-CLEC12A CDR2 VH domain
TISSGGAYTFYKDSVKGRFT

SEQ ID NO: 215 - protein, 20 aa, synthetic construct
  REGION 1..20: note = Description of sequence: anti-CLEC12A CDR2 VH domain
YISGGGTNIYYSDTVKGRFT

SEQ ID NO: 216 - protein, 16 aa, synthetic construct
  REGION 1..16: note = Description of sequence: anti-CLEC12A CDR3 VH domain
ARHSGYDGYYLYAMDY

SEQ ID NO: 217 - protein, 14 aa, synthetic construct
  REGION 1..14: note = Description of sequence: anti-CLEC12A CDR3 VH domain
HSGYDGYYLYAMDY

SEQ ID NO: 218 - protein, 12 aa, synthetic construct
  REGION 1..12: note = Description of sequence: anti-CLEC12A CDR3 VH domain
PNYNYGGSWFAY

SEQ ID NO: 219 - protein, 5 aa, synthetic construct
  REGION 1..5: note = Description of sequence: anti-CLEC12A CDR1 VL domain
SSVHY

SEQ ID NO: 220 - protein, 9 aa, synthetic construct
  REGION 1..9: note = Description of sequence: anti-CLEC12A CDR1 VL domain
ASSSVHYMH

SEQ ID NO: 221 - protein, 10 aa, synthetic construct
  REGION 1..10: note = Description of sequence: anti-CLEC12A CDR1 VL domain
SASSSVHYMH

SEQ ID NO: 222 - protein, 7 aa, synthetic construct
  REGION 1..7: note = Description of sequence: anti-CLEC12A CDR2 VL domain
DTSKLAS

SEQ ID NO: 223 - protein, 9 aa, synthetic construct
  REGION 1..9: note = Description of sequence: anti-CLEC12A CDR3 VL domain
QQWTSNPPT